(19) United States
(12) Patent Application Publication
(10) Pub. No.: US 2022/0354888 A1
(43) Pub. Date: Nov. 10, 2022

(54) ANTISENSE OLIGONUCLEOTIDES (ASOs) DESIGNED TO INHIBIT IMMUNE CHECKPOINT PROTEINS

(71) Applicant: Aalborg Universitet, Aalborg Øst (DK)

(72) Inventors: Sakari Kauppinen, Holte (DK); Andreas Petri, Frederiksberg C (DK); Charlotte Albæk Thrue, Carcavelos (PT)

(21) Appl. No.: 16/322,000

(22) PCT Filed: Aug. 3, 2017
(86) PCT No.: PCT/EP2017/069725

§ 371 Date: Jan. 30, 2019

(30) Foreign Application Priority Data
Aug. 3, 2016 (DK) PA201670576
May 4, 2017 (DK) PA201770309

(51) Int. Cl. A61K 35/17; C12N 5/0783; C12N 15/113; A61P 35/00

(52) U.S. Cl. CPC A61K 35/17; C12N 5/0636; C12N 15/1138; A61P 35/00; C12N 2310/11; C12N 2310/341; C12N 2310/315; C12N 2320/30

(57) ABSTRACT

The present invention provides antisense oligonucleotides directed against immune checkpoints and methods and compositions of using such antisense oligonucleotides for the treatment of cancer.

### FIELD OF THE INVENTION

The present invention relates to compounds and compositions capable of modulating the expression of immune checkpoint proteins in patients or in immune cells ex vivo. In particular, the invention provides antisense oligonucleotide compounds capable of modulating the expression at least one immune checkpoint protein in a patient or in isolated immune cells ex vivo.

### BACKGROUND

Recognition and elimination of cancer cells by the host immune system requires a series of events coordinated by cells of the innate and adaptive immune systems. However, most tumors evade the host immune system by co-opting immune checkpoint pathways, such as the cytotoxic T-lymphocyte-associated protein 4 (CTLA-4) and Programmed Death 1 (PD-1) pathways, respectively, as a key mechanism of immune resistance, especially against T cells that are specific for tumor antigens (Pardoll 2012, Nat Rev Cancer 12:252-264; Topalian et al. 2015, Cancer Cell 27: 450-461). CTLA-4 is upregulated on naïve T cells by antigenic stimulus, and controls the function of regulatory T cells and the establishment of peripheral T cell tolerance. The PD-1 pathway is important for chronic antigenic stimulation of T cells. The engagement of checkpoint receptors on the surface of T cells by their cognate ligands (B7-1 and B7-2 ligands for CTLA-4, PD-L1 and PD-L2 ligands for PD-1) leads to downregulation of T cell function. Binding of PD-L1 and PD-L2 to PD-1 results in decreased T cell proliferation, cytotoxicity, and cytokine production, and increased susceptibility to apoptosis. This plays an important role in the generation and maintenance of peripheral tolerance (Pardoll 2012, Nat Rev Cancer 12:252-64; Topalian et al. 2015, Cancer Cell 27:450-61).

Monoclonal antibodies directed against the receptors or ligands of the immune checkpoint pathways can reverse tumor-induced downregulation of T cell function and unleash antitumor immune activity, leading to tumor regression (Mahoney et al. 2015, Nat Rev Drug Dis 14:561-84; Topalian et al. 2015, Cancer Cell 27: 450-61; Hoos 2016, Nat Rev Drug Dis 15:235-47). The clinical development of drugs that interrupt immune checkpoints has been pioneered by the monoclonal antibody ipilimumab, which blocks CTLA-4 and is now approved for treatment of advanced melanoma on the basis of its survival benefit (Hodi et al. 2010, N Engl J Med 363: 711-23; Robert et al. 2011, N Engl J Med 364:2517-26). Subsequent clinical trials with monoclonal antibodies blocking PD-1 and its ligand PD-L1 have demonstrated good response rates, sustained clinical benefits with encouraging survival rates and good tolerability across many cancer types, most notably advanced non-small cell lung cancer (Topalian et al. 2012, N Engl J Med 366:2443-64; Robert et al. 2015, N Engl J Med 372:2521-32; Hoos 2016, Nat Rev Drug Dis 15:235-47). However, the clinical benefit of these drugs as single agents has been limited to subsets of patients and has not been observed in all tumor types (Mahoney et al. 2015, Nat Rev Drug Dis 14:561-84; Topalian et al. 2015, Cancer Cell 27: 450-61; Hoos 2016, Nat Rev Drug Dis 15:235-47). These limitations call for the development of new therapeutic approaches directed against the expanding inventory of immune checkpoints and new combination therapies, which collectively aim at extending the therapeutic benefits of immune checkpoint blockade to reach a larger proportion of cancer patients.

### Sequence Listing

The present application is being filed along with a sequence listing in electronic format, and is provided as a file named seqListing_ST25_win.txt created on Aug. 2, 2017, which is 1.07 MB (bytes) in size. The disclosure in the electronic format of the sequence listing is incorporated herein by reference in its entirety.

### SUMMARY OF THE INVENTION

The present invention provides novel antisense oligonucleotides directed against immune checkpoints and methods and compositions of using such antisense oligonucleotides for the treatment of cancer.

### BRIEF DESCRIPTION OF THE DRAWINGS

FIG. 1 shows knockdown of CTLA-4 mRNA in the chronic myelogenous leukemia cell line K562 after unassisted uptake of CTLA-4 antisense oligonucleotides CRM0095 and CRM0096, as compared to scrambled control oligo CRM0023 and mock transfection. 1, 0.25 and 0.1 μM of the oligonucleotides were tested.

FIG. 2 shows knockdown of PDCD1 mRNA in the chronic myelogenous leukemia cell line K562 after unassisted uptake of PDCD1 antisense oligonucleotides CRM0097 and CRM0098, as compared to scrambled control oligo CRM0023 and mock transfection. 1, 0.25 and 0.1 μM of the oligonucleotides were tested.

FIG. 3 shows knockdown of CTLA-4 mRNA in the chronic myelogenous leukemia cell line K562 after unassisted uptake of CTLA-4 antisense oligonucleotides CRM0095 and CRM0096, as compared to scrambled control oligo CRM0023 and mock transfection. 2.5 and 0.5 μM of the oligonucleotides were tested.

FIG. 4 shows knockdown of PDCD1 mRNA in the chronic myelogenous leukemia cell line K562 after unassisted uptake of PDCD1 antisense oligonucleotides CRM0097 and CRM0098, as compared to scrambled control oligo CRM0023 and mock transfection. 2.5 and 0.5 μM of the oligonucleotides were tested.

FIG. 5 shows knockdown of PDL1, IDO1, and PDL2 mRNA in GMS-10 cells after lipofectamine-assisted uptake with antisense oligonucleotide CRM0193 targeting both PDL1 and IDO1, or antisense oligonucleotide CRM0196 targeting both PDL1 and PDL2, or antisense oligonucleotide CRM0198 targeting both IDO1 and PDL2, as compared with Scrambled oligonucleotide control CRM0023 and mock transfection. Antisense oligonucleotide concentration was 25 nM and incubation time 24 hours.

FIG. 6A shows PDL1 protein downregulation in GMS-10 cells after lipofectamine-assisted uptake of antisense oligonucleotide CRM0193 targeting both PDL1 and IDO1, or antisense oligonucleotide CRM0196 targeting both PDL1 and PDL2, or antisense oligonucleotide CRM0198 targeting both IDO1 and PDL2, as compared with mock transfection. Antisense oligonucleotide concentration was 25 nM and incubation time 48 hours.

FIG. 6B shows PDL1 protein downregulation in GMS-10 after lipofectamine-assisted uptake of antisense oligonucleotide CRM0185 targeting PDL1, or antisense oligonucleotide CRM0187 targeting IDO1, or antisense oligonucleotide CRM0190 targeting PDL2, as compared with mock transfection. Antisense oligonucleotide concentration was 25 nM and incubation time 48 hours.

FIG. 7 shows knockdown of PDL1, IDO1, and PDL2 mRNA in GMS-10 cells after lipofectamine-assisted uptake of antisense oligonucleotide CRM0185, targeting PDL1, or antisense oligonucleotide CRM0187 targeting IDO1, or antisense oligonucleotide CRM0190 targeting PDL2 as compared with Scrambled oligonucleotide CRM0023 and mock transfection. Antisense oligonucleotide concentration was 25 nM and incubation time 24 hours.

FIG. 8 shows IDO1 protein downregulation in GMS-10 cells after lipofectamine-assisted uptake of antisense oligonucleotide CRM0187 targeting IDO1.

FIG. 9 shows knockdown of PDL1, PDL2, and IDO1 after unassisted delivery of antisense oligonucleotide CRM0185 targeting PDL1, or antisense oligonucleotide CRM0187 targeting IDO1, or antisense oligonucleotide CRM0190 targeting PDL2 into GMS-10 cells . Following knockdown with each antisense oligonucleotide, the expression levels of PDL1, IDO1, and PDL2, respectively, were assessed with qPCR (PDL1=1^{st}, IDO1=2^{nd}, and PDL2=3^{rd} bar in each triplet of bars).

FIG. 10 shows IDO1 protein downregulation in GMS-10 cells after unassisted uptake of antisense oligonucleotide CRM0187 targeting IDO1.

FIG. 11 shows IDO1 protein downregulation in GMS-10 cells after lipofectamine-assisted uptake of antisense oligonucleotide CRM0193 targeting both PDL1 and IDO1, or antisense oligonucleotide CRM0198 targeting both IDO1 and PDL2.

FIG. 12 shows knockdown of PDL1, PDL2, and IDO1 mRNA in GMS-10 cells after lipofectamine-assisted uptake of antisense oligonucleotides CRM0129 or CRM0131, targeting both human and mouse PDL1, or antisense oligonucleotides CRM0134 or CRM0135 targeting both human and mouse IDO1, or antisense oligonucleotides CRM0138 and CRM0139 targeting both human and mouse PDL2 as compared with scrambled oligonucleotide CRM0023 and mock transfection. The expression levels of PDL1, IDO1, and PDL2 were assessed with qPCR (PDL1=1^{st}, IDO1=2^{nd}, and PDL2=3^{rd} bar in each triplet of bars).

FIG. 13 shows knockdown of PDL1 mRNA in murine Neuro-2a cells after lipofectamine-assisted uptake of antisense oligonucleotides CRM0129 or CRM0131, targeting both human and mouse PDL1.

FIG. 14 shows downregulation of IDO1 protein levels in GMS-10 cells after lipofectamine-assisted uptake of antisense oligonucleotides CRM0129 or CRM0131, targeting both human and mouse PDL1, or antisense oligonucleotides CRM0134 or CRM0135 targeting both human and mouse IDO1, or antisense oligonucleotide CRM0138 targeting both human and mouse PDL2 as compared with scrambled oligonucleotide CRM0023 and mock transfection.

### DETAILED DESCRIPTION OF THE INVENTION

### Terms and Definitions

In describing the embodiments of the invention specific terminology will be resorted to for the sake of clarity. However, the invention is not intended to be limited to the specific terms so selected, and it is understood that each specific term includes all technical equivalents, which operate in a similar manner to accomplish a similar purpose.

The term “therapeutically effective amount”, or “effective amount” or effective dose”, refers to an amount of a therapeutic agent, which confers a desired therapeutic effect on an individual in need of the agent. The effective amount may vary among individuals depending on the health and physical condition of the individual to be treated, the taxonomic group of the individuals to be treated, the formulation of the composition, the method of administration, assessment of the individual's medical condition, and other relevant factors.

The term “treatment” refers to any administration of a therapeutic medicament, herein comprising an antisense oligonucleotide that partially or completely cures or reduces one or more symptoms or features of a given disease.

The term “compound” as used herein, refers to a compound comprising an oligonucleotide according to the invention. In some embodiments, a compound may comprise other elements a part from the oligonucleotide of the invention. Such other elements may in non-limiting example be a delivery vehicle which is conjugated or in other way bound to the oligonucleotide.

“Antisense oligonucleotide” means a single-stranded oligonucleotide having a nucleobase sequence that permits hybridization to a corresponding region or segment of a target nucleic acid. The antisense oligonucleotide of the present invention is preferably a gapmer.

A “gapmer” is a chimeric antisense compound, in which an internal region having a plurality of nucleosides (such as a region of at least 6 or 7 DNA nucleotides), which is capable of recruiting an RNAse, such as RNAseH, which region is positioned between external wings at each end, having one or more nucleosides, wherein the nucleosides comprising the internal region are chemically distinct from the nucleoside or nucleosides comprising the external wings.

The internal region of a gapmer may be referred to as the “gap”.

The external regions of a gapmer may be referred to as the “wings”.

“Nucleoside analogues” are described by e.g. Freier & Altmann; Nucl. Acid. Res., 1997, 25, 4429-4443 and Uhlmann; Curr. Opinion in Drug Development, 2000, 3(2), 293-213, and examples of suitable and preferred nucleoside analogues are provided by WO2007031091, which are hereby incorporated by reference.

“5-methylcytosine” means a cytosine modified with a methyl group attached to the 5′ position. A 5-methylcytosine is a modified nucleobase.

“2′-O-methoxyethyl” (also 2′-MOE and 2′-O(CH˜)˜—OCH3) refers to an O-methoxy-ethyl modification at the 2′ position of a furanose ring.

“2′-MOE nucleoside” (also 2′-O-methoxyethyl nucleoside) means a nucleoside comprising a 2′-MOE modified sugar moiety.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides. In some aspects bicyclic nucleoside analogues are LNA nucleotides, and these terms may therefore be used interchangeably, and in such embodiments, both are characterized by the presence of a linker group (such as a bridge) between C2′ and C4′ of the ribose sugar ring. When used in the present context, the terms “LNA unit”, “LNA monomer”, “LNA residue”, “locked nucleic acid unit”, “locked nucleic acid monomer” or “locked nucleic acid residue”, refer to a bicyclic nucleoside analogue. LNA units are described in inter alia WO 99/14226, WO 00/56746, WO 00/56748, WO 01/25248, WO 02/28875, WO 03/006475, WO2015071388, and WO 03/095467.

“Beta-D-Oxy LNA”, is a preferred LNA variant.

“Bicyclic nucleic acid” or “BNA” or “BNA nucleosides” mean nucleic acid monomers having a bridge connecting two carbon atoms between the 4′ and 2′ position of the nucleoside sugar unit, thereby forming a bicyclic sugar. Examples of such bicyclic sugar include, but are not limited to A) pt-L-methyleneoxy (4′-CH2-0-2′) LNA, (B) P-D-Methyleneoxy (4′-CH2-0-2′) LNA, (C) Ethyleneoxy (4′-(CH2)2-0-2′) LNA, (D) Aminooxy (4′-CH2-0-N(R)-2′) LNA and (E) Oxyamino (4′-CH2-N(R)-0-2′) LNA.

As used herein, LNA compounds include, but are not limited to, compounds having at least one bridge between the 4′ and the 2′ position of the sugar wherein each of the bridges independently comprises 1 or from 2 to 4 linked groups independently selected from —[C(R˜)(R2)],—, —C(R˜)═C(R2)-, —C(R˜)═N, —C(═NREM)-, —C(=0)-, —C(═S)—, -0-, —Si(Ri)q-, —S(=0)- and —N(R&)-; wherein: x is 0, 1, or 2; n is 1, 2, 3, or 4; each R& and R2 is, independently, H, a protecting group, hydroxyl, C»C» alkyl, substituted C» (—CHz-) group connecting the 2′ oxygen atom and the 4′ carbon atom, for which the term methyleneoxy (4′-CH&-0-2′) LNA is used.

Furthermore; in the case of the bicyclic sugar moiety having an ethylene bridging group in this position, the ethyleneoxy (4′-CH&CH&-0-2′) LNA is used. n -L-methyleneoxy (4′-CH&-0-2′), an isomer of methyleneoxy (4′-CH&-0-2′) LNA is also encompassed within the definition of LNA, as used herein.

In some embodiments, the nucleoside unit is an LNA unit selected from the list of beta-D-oxy-LNA, alpha-Loxy-LNA, beta-D-amino-LNA, alpha-L-amino-LNA, beta-D-thio-LNA, alpha-L-thio-LNA, 5′-methyl-LNA, beta-D-ENA and alpha-L-ENA.

“cEt” or “constrained ethyl” means a bicyclic sugar moiety comprising a bridge connecting the 4′-carbon and the 2′-carbon, wherein the bridge has the formula: 4′-CH(CHq)-0-2′.

“Constrained ethyl nucleoside” (also cEt nucleoside) means a nucleoside comprising a bicyclic sugar moiety comprising a 4′-CH(CH3)-0-2′ bridge. cEt and some of its properties are described in Pallan et al. Chem Commun (Camb). 2012, Aug. 25; 48(66): 8195-8197.

“Tricyclo (tc)-DNA” belongs to the class of conformationally constrained DNA analogs that show enhanced binding properties to DNA and RNA. Structure and method of production may be seen in Renneberg et al. Nucleic Acids Res. 2002 Jul. 1; 30(13): 2751-2757.

“2′-fluoro”, as referred to herein is a nucleoside comprising a fluoro group at the 2′ position of the sugar ring. 2′-fluorinated nucleotides are described in Peng et al. J Fluor Chem. 2008 September; 129(9): 743-766.

“2′-O-methyl”, as referred to herein, is a nucleoside comprising a sugar comprising an —OCH3 group at the 2′ position of the sugar ring.

“Conformationally Restricted Nucleosides (CRN)” and methods for their synthesis, as referred to herein, are described in WO2013036868, which is hereby incorporated by reference. CRN are sugar-modified nucleosides, in which, similar to LNA, a chemical bridge connects the C2′ and C4′ carbons of the ribose. However, in a CRN, the C2′-C4′ bridge is one carbon longer than in an LNA molecule. The chemical bridge in the ribose of a CRN locks the ribose in a fixed position, which in turn restricts the flexibility of the nucleobase and phosphate group. CRN substitution within an RNA- or DNA-based oligonucleotide has the advantages of increased hybridization affinity and enhanced resistance to nuclease degradation.

“Unlocked Nucleic Acid” or “UNA”, is as referred to herein unlocked nucleic acid typically where the C2-C3 C-C bond of the ribose has been removed, forming an unlocked “sugar” residue (see Fluiter et al., Mol. Biosyst., 2009, 10, 1039, hereby incorporated by reference, and Snead et al. Molecular Therapy—Nucleic Acids (2013) 2, e103;).

“Cancer” is also known as malignant neoplasm, which is a term for diseases, in which abnormal cells divide without control, and can invade nearby tissues or spread to other parts of the body.

“Hepatocellular carcinoma” (HCC) is the most common type of liver cancer. Carcinoma means that it is a cancer found in tissues that cover or line the surfaces of the liver. This is the most common liver cancer type. Internucleoside linkages are in preferred embodiments phosphorothioate linkages, however, it is recognized that the inclusion of phosphodiester linkages, such as one or two linkages, into an otherwise phosphorothioate oligonucleotide, particularly between or adjacent to nucleotide analogue units can modify the bioavailability and/or bio-distribution of an oligonucleotide as described in WO2008/053314, hereby incorporated by reference. In some embodiments, where suitable and not specifically indicated, all remaining linkage groups are either phosphodiester or phosphorothioate, or a mixture thereof.

The term “ex vivo treatment of cells” with oligonucleotides, includes administration to the cells ex vivo of an oligonucleotide capable of targeting and inhibiting the expression of immune checkpoint proteins on antigen presenting cells (APC) or on T cells (ligands). This provides the opportunity to selectively affect expression of a gene in a desired target cell. Well known transfection methods such as lipid based or vector (e.g. viral) based may be used to facilitate uptake of the oligonucleotides in the cells ex vivo.

The term “unassisted uptake” refers to a transfection method, in which antisense oligonucleotides are delivered to cells essentially as described in Soifer et al. (Methods Mol Biol. 2012; 815: 333-46).

The term “GalNAc” or “GalNAc Conjugate” moieties as referred to herein are galactose derivatives, preferably an N-acetylgalactosamine (GalNAc) conjugate moiety. More preferably a trivalent N-acetylgalactosamine moiety is used. GalNAc conjugation of antisense oligonucleotides is known previously as described in WO2015071388. Targeting to hepatocytes in the liver can be greatly enhanced by the addition of a conjugate moiety.

“Target region” means a portion of a target nucleic acid to which one or more antisense compounds is targeted.

“Targeted delivery” as used herein means delivery, wherein the antisense oligonucleotide has either been formulated in a way that will facilitate efficient delivery in specific tissues or cells, or wherein the antisense oligonucleotide in other ways has been for example modified to comprise a targeting moiety, or in other way has been modified in order to facilitate uptake in specific target cells.

The term “Immune Checkpoint Protein” as used herein, refers to certain molecules expressed either by T-cells (receptors) of the immune system, or by antigen presenting cells (APC) in the body (ligands). Immune Checkpoint Proteins are used by the T-cells to identify if a cell is normal and healthy or infected or cancerous. Cancer cells often use expression of Immune Checkpoint Proteins to evade an immune response against them. Use of antibodies to inhibit the interaction between the Immune Checkpoint Protein receptor on T-cells and its ligand on antigen presenting cells or tumor cells has proved effective in cancer treatment.

The antisense oligonucleotides of the invention are designed to target immune checkpoint proteins on antigen presenting cells (APC), tumor cells or on T cells:

Specific antisense oligonucleotides have been designed to target regions of the mRNA coding for the following Immune Checkpoint Proteins on APC or tumor cells:

“CD274”, which is also sometimes termed “PDL1”, and as used herein has Ensembl gene id: ENSG00000120217 and Ensembl transcript id: ENST00000381577. The mouse version of CD274 is termed “Cd274”, and has Ensembl gene id (mouse): ENSMUSG00000016496, and Ensembl transcript id: ENSMUST00000016640.

“PDCD1LG2”, which is also sometimes termed “PDL2”, and as used herein has Ensembl gene id: ENSG00000197646 and Ensembl transcript id: ENST00000397747. The mouse version of PDCD1LG2 is termed “Pdcd1lg2”, and has Ensembl gene id (mouse): ENSMUSG00000016498, and Ensembl transcript id: ENSMUST00000112576.

“CD80”, as used herein has Ensembl gene id: ENSG00000121594 and Ensembl transcript id: ENST00000264246. The mouse version of CD80 is termed “Cd80”, and has Ensembl gene id (mouse): ENSMUSG00000075122, and Ensembl transcript id: ENSMUST00000099816.

“CD86”, as used herein has Ensembl gene id: ENSG00000114013 and Ensembl transcript id: ENST00000330540. The mouse version of CD86 is termed “Cd86”, and has Ensembl gene id (mouse): ENSMUSG00000022901, and Ensembl transcript id: ENSMUST00000089620.

“CD276” which is also sometimes termed “B7-H3”, and as used herein has Ensembl gene id: ENSG00000103855 and Ensembl transcript id: ENST00000318443. The mouse version of CD276 is termed “Cd276”, and has Ensembl gene id (mouse): ENSMUSG00000035914, and Ensembl transcript id: ENSMUST00000165365.

“VTCN1” which is also sometimes termed “B7-H4”, and as used herein has Ensembl gene id: ENSG00000134258 and Ensembl transcript id: ENST00000369458. The mouse version of VTCN1 is termed “Vtcn1”, and has Ensembl gene id (mouse): ENSMUSG00000051076, and Ensembl transcript id: ENSMUST00000054791.

“TNFRSF14” which is also sometimes termed “HVEM”, and as used herein has Ensembl gene id: ENSG00000157873 and Ensembl transcript id: ENST00000355716. The mouse version of TNFRSF14 is termed “Tnfrsf14”, and has Ensembl gene id (mouse): ENSMUSG00000042333, and Ensembl transcript id: ENSMUST00000123514.

“LGALS9” which is also sometimes termed “GAL9”, and as used herein has Ensembl gene id: ENSG00000168961 and Ensembl transcript id: ENST00000395473. The mouse version of LGALS9 is termed “Lgals9”, and has Ensembl gene id (mouse): ENSMUSG00000001123, and Ensembl transcript id: ENSMUST00000108268.

“IDO1”, as used herein has Ensembl gene id: ENSG00000131203 and Ensembl transcript id: ENST00000518237. The mouse version of IDO1 is termed “Ido1”, and has Ensembl gene id (mouse): ENSMUSG00000031551, and Ensembl transcript id: ENSMUST00000033956.

“HMOX1” which is also sometimes termed “HO1”, and as used herein has Ensembl gene id: ENSG00000100292 and Ensembl transcript id: ENST00000216117. The mouse version of HMOX1 is termed “Hmox1”, and has Ensembl gene id (mouse): ENSMUSG00000005413, and Ensembl transcript id: ENSMUST00000005548.

Specific oligonucleotides have been designed which target regions of the mRNA coding for the following T cell receptors:

“PDCD1” which is also sometimes termed “PD1”, and as used herein has Ensembl gene id: ENSG00000188389 and Ensembl transcript id: ENST00000334409. The mouse version of PDCD1 is termed “Pdcd1”, and has Ensembl gene id (mouse): ENSMUSG00000026285, and Ensembl transcript id: ENSMUST00000027507.

“CTLA4” as used herein has Ensembl gene id: ENSG00000163599 and Ensembl transcript id: ENST00000302823. The mouse version of CTLA4 is termed “Ctla4”, and has Ensembl gene id (mouse): ENSMUSG00000026011, and Ensembl transcript id: ENSMUST00000027164.

“LAG3” as used herein has Ensembl gene id: ENSG00000089692 and Ensembl transcript id: ENST00000203629. The mouse version of LAG3 is termed “Lag3”, and has Ensembl gene id (mouse): ENSMUSG00000030124, and Ensembl transcript id: ENSMUST00000032217.

“HAVCR2” as used herein has Ensembl gene id: ENSG00000135077 and Ensembl transcript id: ENST00000307851. The mouse version of HAVCR2 is termed “Havcr2”, and has Ensembl gene id (mouse): ENSMUSG00000020399, and Ensembl transcript id: ENSMUST00000020668.

“TDO2” as used herein has Ensembl gene id: ENSG00000151790 and Ensembl transcript id: ENST00000536354. The mouse version of TDO2 is termed “Tdo2”, and has Ensembl gene id (mouse): ENSMUSG00000028011, and Ensembl transcript id: ENSMUST00000029645.

“TIGIT as used herein has Ensembl gene id: ENSG00000181847 and Ensembl transcript id: ENST00000486257. The mouse version of TIGIT is termed “Tigit”, and has Ensembl gene id (mouse): ENSMUSG00000071552, and Ensembl transcript id: ENSMUST00000096065.

“VSIR” as used herein has Ensembl gene id: ENSG00000107738 and Ensembl transcript id: ENST00000394957. The mouse version of VSIR is termed “Vsir”, and has Ensembl gene id (mouse): ENSMUSG00000020101, and Ensembl transcript id: ENSMUST00000020301.

“CEACAM1” as used herein has Ensembl gene id: ENSG00000079385 and Ensembl transcript id: ENST00000161559. The mouse version of CEACAM1 is termed “Ceacam1”, and has Ensembl gene id (mouse): ENSMUSG00000074272, and Ensembl transcript id: ENSMUST00000098666.

“NT5E” as used herein has Ensembl gene id: ENSG00000135318 and Ensembl transcript id: ENST00000257770. The mouse version of NT5E is termed “Nt5e”, and has Ensembl gene id (mouse): ENSMUSG00000032420, and Ensembl transcript id: ENSMUST00000034992.

“KIR2DL1” as used herein has Ensembl gene id: ENSG00000125498 and Ensembl transcript id: ENST00000336077.

“KIR2DL3” as used herein has Ensembl gene id: ENSG00000243772 and Ensembl transcript id: ENST00000342376.

The above reference to Ensembl gene or transcript id's are according to Ensembl release 89.

### Compounds and Compositions

The present invention relates to chemically-modified antisense oligonucleotides (ASOs) designed to modulate one or more Immune Checkpoint Protein mRNAs, for treatment of human disease, such as cancer or infectious diseases.

The ASOs of the present invention recruit RNase H activity for degradation of the target mRNA, and optionally comprise phosphorothioate internucleotide linkages, to enhance their pharmacokinetic properties in vivo.

Suitably, the antisense oligonucleotides of the invention are capable of down-regulating or modulating their targets, i.e. an Immune Checkpoint Protein-encoding mRNA. The invention provides specific antisense oligonucleotides targeting one, two or three immune checkpoint proteins simultaneously. Further, compositions are provided comprising one or more antisense oligonucleotides according to the invention, whereby the composition is capable of targeting from 1 to 10 immune checkpoint protein coding mRNAs.

If more than one Immune Checkpoint Protein is inhibited by a composition, an additive or synergistic effect may be achieved on the disease. The effect may be symptomatic or may even be curative, i.e. in a cancer patient all cancer cells might be killed.

Therefore, in some preferred embodiments, the antisense oligonucleotides or compositions of the invention are capable of down-regulating or modulating more than one Immune Checkpoint Protein encoding mRNA in a cell. In some embodiments, the invention provides a composition comprising one or more antisense oligonucleotides according to the invention, wherein the composition is capable of down-regulating or modulating more than one Immune Checkpoint Protein encoding mRNA in a cell. In some embodiments, the invention provides a composition comprising one or more antisense oligonucleotides according to the invention, wherein the composition when administered to a cell in vivo or ex vivo, is capable of down-regulating or modulating one Immune Checkpoint Protein encoding mRNA in the cell. In some embodiments, the invention provides a composition comprising one or more antisense oligonucleotides according to the invention, wherein the composition when administered to a cell is capable of down-regulating or modulating two different Immune Checkpoint Protein encoding mRNAs in the cell. In some embodiments, the invention provides a composition comprising one or more antisense oligonucleotides according to the invention, wherein the composition when administered to a cell in vitro or in vivo, is capable of down-regulating or modulating three different Immune Checkpoint Protein encoding mRNAs in the cell. In some embodiments, the invention provides a composition comprising one or more antisense oligonucleotides according to the invention, wherein the composition when administered to a cell ex vivo or in vivo, is capable of down-regulating or modulating four different Immune Checkpoint Protein encoding mRNAs in the cell. In some embodiments, the invention provides a composition comprising one or more antisense oligonucleotides according to the invention, wherein the composition when administered to a cell ex vivo or in vivo, is capable of down-regulating or modulating five different Immune Checkpoint Protein encoding mRNAs in the cell. In some embodiments, the invention provides a composition comprising one or more antisense oligonucleotides according to the invention, wherein the composition when administered to a cell in vitro or in vivo, is capable of down-regulating or modulating six different Immune Checkpoint Protein encoding mRNAs in the cell. In some embodiments, the invention provides a composition comprising one or more antisense oligonucleotides according to the invention, wherein the composition when administered to a cell ex vivo or in vivo, is capable of down-regulating or modulating seven, eight, nine or ten different Immune Checkpoint Protein mRNAs in the cell.

In some embodiments, it may be an advantage to target not only the immune checkpoint receptor on T cells, but also its ligand on antigen presenting cells (APC) or tumor cells, to achieve a more efficient treatment of the disease. Therefore, in some preferred embodiments, the invention provides compositions comprising one or more antisense oligonucleotides according to the invention, wherein the composition is capable of targeting both a immune checkpoint receptor and its ligand.

In order to be able to provide efficient treatment, the present invention provides antisense oligonucleotides consisting of a sequence of 14-22 nucleobases in length that is a gapmer comprising a central region of 6 to 16 consecutive DNA nucleotides flanked in each end by wing regions each comprising 1 to 5 nucleotide analogues, wherein the oligonucleotide is complementary to an mRNA encoding an immune checkpoint protein.

In order to ensure efficient treatment using the antisense oligonucleotides of the invention, when used in vivo, the stability of the oligonucleotides may be improved by introduction of alternatives to the normal phosphodiester internucleotide bonds. In some embodiments, the antisense oligonucleotides of the invention comprise one or more phosphorothioate internucleotide linkages. In preferred embodiments, the antisense oligonucleotide according to the invention comprises 1 to 21 phosphorothioate internucleotide linkages. Certain immune checkpoint proteins are of particular interest for use in cancer treatment. In some embodiments, the antisense oligonucleotide according to the invention is complementary to a region of the mRNA encoding anyone of the immune checkpoint proteins selected from the list of CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, and KIR2DL3. In some embodiments, the antisense oligonucleotides or compositions are capable of downregulating or modulating one or more immune checkpoint proteins. In some instances, an antisense oligonucleotide according to the invention is capable of downregulating or modulating the expression of one, two or three immune checkpoint proteins selected from the list of CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, and KIR2DL3. In some instances the compositions comprising antisense oligonucleotides of the invention are capable of downregulating or modulating the expression of one or more immune checkpoint proteins selected from the list of CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, and KIR2DL3. Accordingly, in some embodiments, the antisense oligonucleotide according to the invention is complementary to a region of at least one, such as one mRNA selected from the group consisting of an mRNA encoding CD274, an mRNA encoding PDCD1LG2, an mRNA encoding CD80, an mRNA encoding CD86, an mRNA encoding CD276, an mRNA encoding VTCN1, an mRNA encoding TNFRSF14, an mRNA encoding LGALS9, an mRNA encoding IDO1, mRNA encoding HMOX1, an mRNA encoding PDCD1, an mRNA encoding CTLA4, an mRNA encoding LAG3, an mRNA encoding HAVCR2, an mRNA encoding TDO2, an mRNA encoding TIGIT, an mRNA encoding VSIR, an mRNA encoding CEACAM1, an mRNA encoding NT5E, an mRNA encoding KIR2DL1, and an mRNA encoding KIR2DL3.

In some embodiments, the antisense oligonucleotide of the invention is complementary to a region of at least two, such as two mRNAs selected from the group consisting of an mRNA encoding CD274, an mRNA encoding PDCD1LG2, an mRNA encoding CD80, an mRNA encoding CD86, an mRNA encoding CD276, an mRNA encoding VTCN1, an mRNA encoding TNFRSF14, an mRNA encoding LGALS9, an mRNA encoding IDO1, mRNA encoding HMOX1, an mRNA encoding PDCD1, an mRNA encoding CTLA4, an mRNA encoding LAG3, an mRNA encoding HAVCR2, an mRNA encoding TDO2, an mRNA encoding TIGIT, an mRNA encoding VSIR, an mRNA encoding CEACAM1, an mRNA encoding NT5E, an mRNA encoding KIR2DL1, and an mRNA encoding KIR2DL3.

In some embodiments, the antisense oligonucleotide according to the invention is complementary to a region of at least three, such as three mRNAs selected from the group consisting of an mRNA encoding CD274, an mRNA encoding PDCD1LG2, an mRNA encoding CD80, an mRNA encoding CD86, an mRNA encoding CD276, an mRNA encoding VTCN1, an mRNA encoding TNFRSF14, an mRNA encoding LGALS9, an mRNA encoding IDO1, mRNA encoding HMOX1, an mRNA encoding PDCD1, an mRNA encoding CTLA4, an mRNA encoding LAG3, an mRNA encoding HAVCR2, an mRNA encoding TDO2, an mRNA encoding TIGIT, an mRNA encoding VSIR, an mRNA encoding CEACAM1, an mRNA encoding NT5E, an mRNA encoding KIR2DL1, and an mRNA encoding KIR2DL3.

Thus, in some embodiments, the antisense oligonucleotide according to the invention is capable of decreasing expression of at least two immune checkpoint proteins selected from of CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, and KIR2DL3. In some embodiments, the antisense oligonucleotide according to the invention is capable of decreasing expression of three immune checkpoint proteins selected CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, and KIR2DL3.

The present invention provides some advantageous target regions in the mRNAs of immune checkpoint proteins CD274, PDCD1LG2, CD80, CD86, CD276, VTCN1, TNFRSF14, LGALS9, IDO1, HMOX1, PDCD1 CTLA4, LAG3, HAVCR2, TDO2, TIGIT, VSIR, CEACAM1, NT5E, KIR2DL1, and KIR2DL3 that are specially preferred, and in some preferred embodiments, the antisense oligonucleotide according to the invention is complementary to anyone of SEQ ID NOs: 1-375, or anyone of SEQ ID NOs: 1473-1503, or anyone of SEQ ID NOs: 1535-1593 or to SEQ ID NO: 1654 or to anyone of SEQ ID NOs: 1655-2001, or to anyone of SEQ ID NOs: 3044-3052, or to anyone of SEQ ID NOs: 3062-3097.

Furthermore, in preferred embodiments, the antisense oligonucleotide of the invention is a gapmer, wherein at least one of the wing regions comprises at least one nucleoside analogue selected from the list of beta-D-oxy LNA, alpha-L-oxy-LNA, beta-D-amino-LNA, alpha-L-amino-LNA, beta-D-thio-LNA, alpha-L-thio-LNA, 5′-methyl-LNA, beta-D-ENA and alpha-L-ENA.

In a particularly preferred embodiment, the antisense oligonucleotide of the invention comprises at least one Beta-D-Oxy LNA nucleotide in the wings. In some embodiments, the antisense oligonucleotides of the invention are provided which do not comprise LNA. In such embodiments, the nucleoside analogue may be selected from the group consisting of tricyclo-DNA, 2′-fluoro, 2′-O-methyl, 2′-methoxyethyl (2′-MOE), 2′cyclic ethyl (cET), and Conformationally Restricted Nucleoside (CRN). In some embodiments, the antisense oligonucleotide according to the invention comprises a mixture of nucleoside analogues, so that at least one nucleoside analogue is not LNA. Accordingly, in some embodiments, the antisense oligonucleotide according to the invention is designed so that at least one of the wing regions comprises two or more nucleoside analogues, wherein said nucleotide analogues is a mixture of LNA and at least one nucleoside analogue independently selected from the group consisting of tricyclo-DNA, 2′-fluoro, 2′-O-methyl, 2′-methoxyethyl (2′-MOE), 2′cyclic ethyl (cET), and Conformationally Restricted Nucleoside (CRN).

In preferred embodiments, the antisense oligonucleotide according to the invention comprises two or more nucleoside analogues which are a mixture of LNA and 2′-fluoro.

The present invention provides a number of specific preferred LNA antisense oligonucleotides targeting one or more of the immune checkpoint proteins from the list CD274, PDCD1LG2, CD80, CD86, CD276, VTCN1, TNFRSF14, LGALS9, IDO1, HMOX1, PDCD1 CTLA4, LAG3, HAVCR2, TDO2, TIGIT, VSIR, CEACAM1, NT5E, KIR2DL1, and KIR2DL3. These antisense oligonucleotides are any one of SEQ ID NOs: 376-1472, or anyone of SEQ ID NOs: 1504-1534, or anyone of SEQ ID NOs: 1594-1653, or anyone of SEQ ID NOs: 2002-3043, or anyone of SEQ ID NOs: 3053-3061, or anyone of SEQ ID NOs: 3098-3133, and their design, sequence and targets are described in Tables 3.1, 3.2, 5.1, 5.2, 7.1 and 7.2.

Accordingly, in one preferred embodiment, the antisense oligonucleotide according to the invention is a compound of ID NO:CRM0193 complementary to and capable of decreasing the expression of the immune checkpoint proteins PDL1 and/or IDO.

In another preferred embodiment the antisense oligonucleotide according to the invention is a compound of ID NO: CRM0296 complementary to and capable of decreasing the expression of the immune checkpoint proteins PDL1 and/or PDL2.

In another preferred embodiment the antisense oligonucleotide according to the invention is a compound of ID NO: CRM0198 complementary to and capable of decreasing the expression of the immune checkpoint proteins PDL2 and/or IDO.

Accordingly, in another preferred embodiment the antisense oligonucleotide according to the invention is a compound of ID NO:CRM0185 complementary to and capable of decreasing the expression of the immune checkpoint protein PDL1.

In another preferred embodiment the antisense oligonucleotide according to the invention is a compound of ID NO:CRM0187 complementary to and capable of decreasing the expression of the immune checkpoint protein IDO. In another preferred embodiment the antisense oligonucleotide according to the invention is a compound of ID NO:CRM0190 complementary to and capable of decreasing the expression of the immune checkpoint protein PDL2.

### Uses of the Antisense Oligonucleotides of the Invention

The antisense oligonucleotides of the invention may be used for in vivo treatment, as well as for ex vivo treatment approaches, such as in cancer vaccine methods. In some embodiments, the use of the antisense oligonucleotides is for generation of compositions for use in in vivo treatment of disease, such as cancer.

### Use in Ex Vivo Methods for Making Anti-Cancer Vaccines

Cancer treatment using adoptive cell transfer methods and dendritic cell based anti-cancer vaccines are rapidly being developed. Adoptive cell transfer in some cases involve genetic modifications of T-cells to express receptors that recognize specific tumor-associated antigens, and which also comprise in the receptor construct costimulatory molecules for activation of the T-cell response. The present invention provides novel methods of modifying ex-vivo expanded T-cells to make them useful as anti-cancer treatment. In some embodiments, the antisense oligonucleotides of the invention may be used ex vivo to modify expanded T-cells by knocking down expression of CTLA4 and/or PDCD1 and/or LAG3 and/or HAVCR2 and/or TIGIT and/or CEACAM1 in order to prevent the T-cells from seeing cancer cells as normal cells, and thereby initiate an immune response against the cancer cells.

In a different approach, the antisense oligonucleotides of the invention may be used to create a novel dendritic cell-based anti-cancer vaccine. T cell responses can be initiated, supported and boosted by dendritic cells. These are “professional” antigen-presenting cells, and can activate T cells upon presentation of a peptide in concordance with co-stimulatory signals, which is dependent on the balance between co-inhibitory and co-stimulatory interactions. PD-L1 (CD274) and PD-L2 (PDCD1LG2) are two of the co-inhibitory ligands that are involved in this process. CD8⁺ T-cells that recognize tumor cells expressing minor histocompatibility antigens (MiHAs) express the receptor (PD1 (PDCD1)) for PD-L1 and PD-L2 after A allogenetic stem cell transplantation. However, the high expression of PD1 in the MiHA-specific CD8⁺ T cells causes a functional inhibition of the T cells due to the interaction between PD1 and its ligands PD-L1 and PD-L2. Thus, the antisense oligonucleotides of the present invention may be used to knock down expression of PDCD1LG1 and/or PDCD1LG2 in isolated and expanded dendritic cells before those are used for the treatment of cancer patients. In some embodiments, the modified dendritic cells are used ex vivo to augment the expansion of MiHA specific CD8⁺ T cells ex vivo. Thus, the present invention provides methods of ex vivo expansion and modulation of T-cells or dendritic cells for use as anti-cancer vaccines. In some embodiments, the antisense oligonucleotides of the invention targeting anyone or both of CTLA4 or PDCD1 are used in ex vivo methods of modifying CTLA4 and/or PDCD1 expression in expanded T-cells for treatment of cancer patients, wherein the modified T-cells are subsequently administered to the cancer patient. In some embodiments, isolated dendritic cells are tested for expression of immune checkpoint proteins selected from the list of CD274, PDCD1LG2, CD80, CD86, CD276, VTCN1, TNFRSF14, LGALS9, IDO1, HMOX1, TDO2, VSIR and NT5E, and subsequently the dendritic cells are modified by antisense oligonucleotides of the invention which are targeted to one or more or all of the immune checkpoint proteins for which the dendritic cells tested positive. When reintroduced into a patient, the modified dendritic cells will be more efficient in inducing a T-cell response against cancer cells than non-modified dendritic cells.

In some embodiments, the antisense oligonucleotides of the invention are targeted to one or more of the immune checkpoint proteins selected from the list of CD274, PDCD1LG2, CD80, CD86, CD276, VTCN1, TNFRSF14, LGALS9, IDO1, HMOX1, TDO2, VSIR and NT5E, and are for use in treatment of cancer in combination with adoptive cell transfer such as modified T-cells wherein the modified T-cells have been treated to reduce expression of one or more of CTLA4 and PDCD1, and/or LAG3 and/or HAVCR2 and/or TIGIT and/or CEACAM1. In some embodiments, antisense oligonucleotides of the invention targeting one or more immune checkpoint protein mRNAs are used to mitigate immune suppression in methods of treating cancer in combination with dendritic cell-based cancer vaccines. In some such embodiments, the antisense oligonucleotides of the invention targeting one or more immune checkpoint protein mRNAs which are used to mitigate immune suppression in methods of treating cancer in combination with dendritic cell based cancer vaccines, are complementary to an mRNA coding for an immune checkpoint protein selected from the list of CD274, PDCD1LG2, CD80, CD86, CD276, VTCN1, TNFRSF14, LGALS9, IDO1, HMOX1, TDO2, VSIR and NT5E.

In some embodiments, the invention provides a method where isolated natural killer cells (NK cells) are tested for expression of KIR2DL1 and/or KIR2DL3. The isolated cells may then be treated ex vivo by antisense oligonucleotides of the invention targeting KIR2DL1 and/or KIR2DL3, thereby knocking down expression of KIR2DL1 and/or KIR2DL3. The ex vivo expanded, treated NK cells may then be used in a method of treating cancer by NK cell-based immune therapy.

In some embodiments, the antisense oligonucleotide, compound or composition according to the invention is complementary to anyone of the target sequences selected from the list of SEQ ID NOs: 1-375, or SEQ ID NOs: 1473-1503 or anyone of SEQ ID NOs: 1535-1593 or to SEQ ID NO: 1654, or to anyone of SEQ ID NOs: 1655-2001, or to anyone of SEQ ID NOs: 3044-3052, or to anyone of SEQ ID NOs: 3062-3097 and is for treatment of a cell ex vivo.

In some embodiments, the antisense oligonucleotide, compound or composition according to the invention is complementary to anyone of the target sequences selected from the list of SEQ ID NOs: 1-375, or SEQ ID NOs: 1473-1503 or anyone of SEQ ID NOs: 1535-1593 or to SEQ ID NO: 1654, or to anyone of SEQ ID NOs: 1655-2001, or to anyone of SEQ ID NOs: 3044-3052, or to anyone of SEQ ID NOs: 3062-3097 and is for treatment of a cell ex vivo, wherein the oligonucleotide has no more than 1, 2 or 3 mismatches to the target sequence.

In some embodiments, the antisense oligonucleotide, compound or composition which is for use in the treatment of a T-cell ex vivo, is complementary to anyone of SEQ ID NOs: 200-208, 240-249, 261-267, 363, 366, 372, 373, 375, 1488-1493, 1497, 1552-1553, 1562-1565, 1577-1580, 1584-1585, 1588-1589, 1592-1593, 1654, 1656-58, 1665-67, 1675, 1677-78, 1684-85, 1687-88, 1692, 1694, 1702, 1705, 1708, 1724, 1728-29, 1741, 1743, 1750, 1753, 1756-60, 1762-65, 1767, 1774-75, 1784-90, 1796, 1799-1801, 1804, 1808, 1813, 1819, 1826-27, 1829, 1831-32, 1843, 1857-58, 1860, 1866-67, 1871-76, 1878-79, 1882-84, 1893-94, 1896-99, 1909-11, 1920-22, 1924, 1926, 1931, 1934, 1938, 1942-43, 1950-51, 1956-57, 1964-65, 1968, 1970, 1973-75, 1979-81, 1991-94, 1997-2001, 3044-46, 3050, 3062-68, 3077-79, and 3089-94. In some embodiments, the antisense oligonucleotide, compound or composition which is for use in the treatment of an antigen presenting cell, such as a dendritic cell ex vivo, is complementary to anyone of SEQ ID NOs: 1-375, or anyone of SEQ ID NOs: 1473-1487, 1494-1496, 1498-1503, 1535-1551, 1554-1561, 1566-1576, 1581-1583, 1586-1587, 1590-1591, 1655, 1659-65, 1668-1752, 1754-83, 1787-88, 1791-1825, 1828, 1830-42, 1844-73, 1877-81, 1885-95, 1900-49, 1952-67, 1969-2001, 3047-49, 3051-52, 3080-88, and 3095-97.

In some embodiments, the antisense oligonucleotide, compound or composition which is for use in the treatment of a NK cell ex vivo, is complementary to anyone of SEQ ID Nos: 1656, 1665-1668, 1699, 1714, 1727, 1730-1731, 1740, 1753, 1784-1786, 1789-1790, 1841, 1868-1869, 1896-1899, 1918, 1927, 1944, 1968, and 3069-3076.

In some embodiments, the antisense oligonucleotide, compound or composition according to the invention, such as anyone of the oligonucleotides selected from the list of SEQ ID NOs: 376-1472, or anyone of SEQ ID NOs: 1504-1534, or anyone of SEQ ID NOs: 1594-1653, or anyone of SEQ ID NOs: 2002-3043, or anyone of SEQ ID NOs: 3053-3061, or anyone of SEQ ID NOs: 3098-3133 is for treatment of a cell ex vivo.

In some embodiments, the antisense oligonucleotide, compound or composition according to the invention, such as anyone of the oligonucleotides selected from the list of SEQ ID NOs: 973-999, 1093-1122, 1156-1176, 1460, 1463, 1466, 1469,-1470, 1472, 1519-1524, 1528, 1611-1612, 1621-1624, 1636-1639, 1643-1644, 1647-1648, or 1651-1653, 2005-13, 2032-40, 2062-64, 2068-73, 2089-94, 2098-2103, 2013-15, 2019-21, 2143-45, 2152-54, 2161-63, 2209-11, 2221-26, 2254-56, 2260-68, 2287-89, 2296-98, 2305-19, 2323-34, 2338-40, 2359-64, 2390-2410, 2426-28, 2435-43, 2450-52, 2462-64, 2477-79, 2495-97, 2516-21, 2525-27, 2531-36, 2567-69, 2609-14, 2618-20, 2634-41, 2660-68, 2672-77, 2684-92, 2715-22, 2726-37, 2763-73, 2798-2806, 2816-18, 2831-33, 2840-48, 2852-54, 2864-69, 2930-35, 2948-50, 2957-65, 2975-83, 2942-44, 3011-22, 3029-43, 3053-55, 3059, 3098-3104, 3113-15, and 3125-30, is for treatment of a cell ex vivo wherein the cell is a T-cell.

In some embodiments, the antisense oligonucleotide, compound or composition according to the invention, such as anyone of the oligonucleotides selected from the list of SEQ ID NOs: 376-1472, or anyone of SEQ ID NOs: 1504-1518, 1525-1527, 1529-1534, 1594-1610, 1613-1620, 1625-1635, 1640-1642, 1645-1646, 1649-1650, 2002-04, 2014-34, 2039-2295, 2299-2389, 2399-2404, 2411-2515, 2522-24, 2528-66, 2570-2665, 2669-81, 2693-2725, 2736-2941, 2945-3043, 3056-58, 3060-61, 3116-24, and 3131-33 is for treatment of a cell ex vivo wherein the cell is an antigen presenting cell, such as a dendritic cell.

In some embodiments, the antisense oligonucleotide, compound or composition according to the invention, such as anyone of the oligonucleotides selected from the list of SEQ ID NOs: 2005-13, 2032-40, 2134-36, 2179-81, 2218-20, 2227-32, 2251-53, 2257-59, 2296-98, 2390-98, 2405-10, 2561-63, 2642-47, 2726-37, 2792-94, 2819-21, 2870-72, 2942-44, 3105-12 is for treatment of a cell ex vivo, wherein the cell is a NK cell.

In some embodiments, the antisense oligonucleotides of the invention are used for treatment of cancer in combination with a cancer vaccine. In some embodiments, the compounds, antisense oligonucleotides, compositions, ex vivo modified cells, and methods of treatment of the invention are for use in the treatment of cancer. In some such embodiments, the cancer is selected from the list of anyone of a cancer including solid tumors such as skin, breast, brain, cervical carcinomas, testicular carcinomas, etc. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple mycloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanihoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerininoma, malignant teratoma), vulva (squamous cell carcinoma, intraepithelial carcinoma, adenocarcinoma, fibrosarcoina, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

Isolation and expansion of T-cells, such as MiHA specific CD8⁺ T cells, and dendritic cells are well known in the art (for example see van der Waart et al. (2015) Cancer Immunol Immunother 64:645-654).

### Modulation of Immune Checkpoint Proteins in Methods of Treatment.

The present invention relates to chemically-modified antisense oligonucleotides (ASOs) designed to modulate one or more Immune Checkpoint Protein encoding mRNAs, for treatment of human disease, such as cancer.

The ASOs of the present invention recruit RNase H activity for degradation of the target mRNA, and comprise phosphorothioate internucleotide linkages, to enhance their pharmacokinetic properties in vivo. These features make the ASO compounds useful in methods of treating patients by delivery of the oligonucleotides to the patient in vivo.

In some embodiments the invention provides, a method of downregulating one or more immune checkpoint proteins in a cell or in a patient, by administration of a therapeutically effective amount of a compound or antisense oligonucleotide according to the invention and which is complementary to the target and selected from the list of CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, and KIR2DL3. In some embodiments, the antisense oligonucleotide used in the method is complementary to anyone of the sequences selected from the list of anyone of SEQ ID NOs: 1-375, or anyone of SEQ ID NOs: 1473-1503, or anyone of SEQ ID NOs: 1535-1593 or to SEQ ID NO: 1654, or to anyone of SEQ ID NOs: 1655-2001, or to anyone of SEQ ID NOs: 3044-3052, or to anyone of SEQ ID NOs: 3062-3097. In some embodiments, the antisense oligonucleotide for use in the method of treatment is selected from the list of SEQ ID NOs: 376-1472, or anyone of SEQ ID NOs: 1504-1534, or anyone of SEQ ID NOs: 1594-1653, or anyone of SEQ ID NOs: 2002-3043, or anyone of SEQ ID NOs: 3053-3061, or anyone of SEQ ID NOs: 3098-3133.

In some embodiments, the method of treatment is used to treat a cell in a human body. In some embodiments, the method of treatment is used to treat a cancer cell in a human body. In some embodiments, the method of treatment is a method of treating cancer, comprising the administration of a therapeutically effective dosage of a compound or antisense oligonucleotide or a composition according to the invention, such as anyone of the oligonucleotides selected from the list of SEQ ID NOs: 376-1472, or anyone of SEQ ID NOs: 1504-1534, or anyone of SEQ ID NOs: 1594-1653, or anyone of SEQ ID NOs: 2002-3043, or anyone of SEQ ID NOs: 3053-3061, or anyone of SEQ ID NOs: 3098-3133.

In some embodiments, the cancer which is treated by the method of treatment is cancer expressing a mRNA coding for an immune checkpoint protein, such as anyone of CD274, PDCD1LG2, CD80, CD86, CD276, VTCN1, TNFRSF14, LGALS9, IDO1, HMOX1, PDCD1, CTLA4, LAGS, HAVCR2, TDO2, TIGIT, VSIR, CEACAM1, NT5E, KIR2DL1, and KIR2DL3. In some embodiments, the antisense oligonucleotides, compounds or compositions according to the invention is for use in methods of treatment of a cancer selected from the list of cancer, including solid tumors such as skin, breast, brain, cervical carcinomas, testicular carcinomas, etc. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple mycloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanihoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerininoma, malignant teratoma), vulva (squamous cell carcinoma, intraepithelial carcinoma, adenocarcinoma, fibrosarcoina, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

In some instances, additive or synergistic effects may be achieved by combining the use of different drugs in methods of treatment. In some embodiments, the methods of treatment using the antisense oligonucleotides of the invention are for use in combination with another compound, composition or method of treatment. In some embodiments, the combination is with an immune checkpoint protein blocking antibody or a composition comprising an immune checkpoint protein blocking antibody or a method of treatment wherein an Immune Checkpoint Protein blocking antibody is used.

In some embodiments, the antisense oligonucleotides of the invention comprising any one of SEQ ID NOs: 376-1472, or anyone of SEQ ID NOs: 1504-1534, or anyone of SEQ ID NOs: 1594-1653, or anyone of SEQ ID

NOs: 2002-3043, or anyone of SEQ ID NOs: 3053-3061, or anyone of SEQ ID NOs: 3098-3133, are for use in combination with another drug or treatment for cancer. In some embodiments, the antisense oligonucleotides of the invention comprising any one of SEQ ID NOs: 376-1472, or anyone of SEQ ID NOs: 1504-1534, or anyone of SEQ ID NOs: 1594-1653, or anyone of SEQ ID NOs: 2002-3043, or anyone of SEQ ID NOs: 3053-3061, or anyone of SEQ ID NOs: 3098-3133, are for use in combination with another active ingredient. The antisense oligonucleotides of the invention may be formulated together with such other ingredient or drug, or they may be formulated separately.

### Dosages and Compositions

The antisense oligonucleotides of the invention may be used in pharmaceutical formulations and compositions, and are for use in treatment of diseases according to the invention. The compounds and compositions will be used in effective dosages, which means in dosages that are sufficient to achieve a desired effect on a disease parameter. The skilled person will without undue burden be able to determine what a reasonably effective dosage is for individual patients.

As explained initially, the antisense oligonucleotides of the invention will constitute suitable drugs with improved properties. The design of a potent and safe drug requires the fine-tuning of various parameters such as affinity/specificity, stability in biological fluids, cellular uptake, mode of action, pharmacokinetic properties and toxicity. Accordingly, in a further aspect the antisense oligonucleotide may be used in a pharmaceutical composition comprising an oligonucleotide according to the invention and a pharmaceutically acceptable diluent, carrier or adjuvant. Preferably said carrier is saline or buffered saline. In a still further aspect the present invention relates to an antisense oligonucleotide according to the present invention for use as a medicament.

As will be understood, dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual oligonucleotides. Generally it can be estimated based on EC₅₀ values found to be effective in vitro and in vivo animal models. In general, dosage is from 0.01 μg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues.

Following successful treatment, it may be desirable to have the patient undergo maintenance therapy to prevent the recurrence of the disease state. As indicated above, the invention also relates to a pharmaceutical composition, which comprises at least one oligonucleotide of the invention as an active ingredient. It should be understood that the pharmaceutical composition according to the invention optionally comprises a pharmaceutical carrier, and that the pharmaceutical composition optionally comprises further active compounds, such as in non-limiting example chemotherapeutic compounds or anticancer vaccines.

The oligonucleotides of the invention can be used “as is” or in form of a variety of pharmaceutically acceptable salts. As used herein, the term “pharmaceutically acceptable salts” refers to salts that retain the desired biological activity of the herein-identified antisense oligonucleotides and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine.

Thus the present invention provides pharmaceutical compositions comprising the antisense oligonucleotide or compound according to the invention and at least one pharmaceutically-acceptable carrier.

In some embodiments, the pharmaceutical composition of the invention comprises 1, 2, 3, 4, 5, 6, 7, 8, 9 or 10 antisense oligonucleotides according to the invention, wherein the antisense oligonucleotides are selected so that the composition target at least two immune checkpoint proteins.

In some embodiments, the pharmaceutical composition according to the invention target any comprises antisense oligonucleotides according to the invention so that the composition is capable of targeting any one of 2, 3, 4, 5, 6, 7, 8, 9 or 10 different immune checkpoint proteins.

In some embodiments, the invention provides a pharmaceutical composition, wherein the composition comprises more than one compound or antisense oligonucleotide according to the invention.

In some embodiments, a pharmaceutical composition is provided comprising two or more antisense oligonucleotides selected from the list of any one of SEQ ID NOs: 376-1472, or anyone of SEQ ID NOs: 1504-1534, or anyone of SEQ ID NOs: 1594-1653, or anyone of SEQ ID NOs: 2002-3043, or anyone of SEQ ID NOs: 3053-3061, or anyone of SEQ ID NOs: 3098-3133, or which are complementary to anyone of SEQ ID NOs: 1-375, or anyone of SEQ ID NOs: 1473-1503, or anyone of SEQ ID NOs: 1535-1593 or to SEQ ID NO: 1654, or to anyone of SEQ ID NOs: 1655-2001, or to anyone of SEQ ID NOs: 3044-3052, or to anyone of SEQ ID NOs: 3062-3097.

In some embodiments, the antisense oligonucleotide, compound or composition of the invention is for use as a medicament.

In some embodiments, the antisense oligonucleotide, compound or composition according to the invention is for use in the treatment of cancer. In some embodiments, the antisense oligonucleotide, compound or composition according to the invention is for treatment of cancer, wherein the cancer is hepatocellular carcinoma.

In some embodiments, the antisense oligonucleotide, compound or composition is for use in the treatment of a human subject.

### Targeted Delivery

When the antisense oligonucleotides of the present invention are for in vivo use in medicine, various means for delivery may be used in order to achieve efficient targeted delivery to cells and tissues.

Targeted delivery of an antisense oligonucleotide is done depending on the target cell or tissue to reach. Such delivery may be modified by conjugation with a ligand in order to facilitate targeted delivery of the antisense oligonucleotide to target cells and tissues. In some embodiments, the antisense oligonucleotides may be formulated in saline for naked delivery. In some embodiments, the antisense oligonucleotide of the invention is conjugated to anyone of folic acid or N-acetylgalactosamine (GalNAc). In some embodiments, the antisense oligonucleotide according to the invention is made for unconjugated delivery in a pharmaceutical composition. In some embodiments, the antisense oligonucleotide according to the invention is formulated in lipid nanoparticles for delivery to cells in vivo or ex vivo.

There are several approaches for oligonucleotide delivery. One approach is to use a nanoparticle formulation, which determines the tissue distribution and the cellular interactions of the oligonucleotide. Another approach is to use a delivery vehicle to enhance the cellular uptake, in one or more embodiment the vehicle is anyone of folic acid or GalNAc. A third delivery approach is wherein the oligonucleotide is made unconjugated for delivery in a pharmaceutical composition.

The various examples of delivery may be carried out as parenteral administration. By “Parenteral administration” means administration through infusion or injection and comprises intravenous administration, subcutaneous administration, intramuscular administration, intracranial administration, intraperitoneal administration or intra-arterial administration.

The various examples of delivery may be carried out as oral or nasal administration. The nanoparticle formulation can be a liposomal formulation and in one embodiment the anionic oligonucleotide is complexed with a cationic lipid thereby forming lipid nanoparticles. Such lipid nanoparticles are useful for treating liver diseases. The nanoparticle formulation can also be a polymeric nanoparticle (Juliano et. Al.; Survey and summary, the delivery of therapeutic oligonucleotides, Nucleic Acids Research, 2016).

The vehicle used in vehicle-conjugated formulation can be e.g. a lipid vehicle or a polyamine vehicle. One example of a polyamine vehicle is GalNAc—a high-affinity ligand for the hepatocyte-specific asialoglycoprotein receptor (ASGPR). GalNAc-conjugated ASOs show enhanced uptake to hepatocytes instead of non-parenchymal cells since after entry into the cells, the ASO is liberated in the liver (Prakash et. al.; Targeted delivery of antisense oligonucleotides to hepatocytes using triantennary N-acetyl galactosamine improves potency 10-fold in mice, Nucleic acids research, 2014, vol. 42, no. 13, 8796-8807). GalNAc conjugated ASOs may also enhance potency and duration of some ASOs targeting human apolipoprotein C-III and human transthyretin (TTR). Folic acid (FA) conjugated ASOs can be used to target the folate receptor that is a cellular surface markers for many solid tumours and myeloid leukemias (Chiu et. al.; Efficient Delivery of an Antisense Oligodeoxyribonucleotide Formulated in Folate Receptor-targeted Liposomes).

In methods using so-called naked delivery, the oligonucleotide is formulated into a solution comprising saline. This approach is effective in many kinds of cell types among others: primary cells, dividing and non-dividing cells (Soifer et. al.; Silencing of Gene Expression by Gymnotic Delivery of Antisense Oligonucleotides; chapter 25; Michael Kaufmann and Claudia Klinger (eds.), Functional Genomics: Methods and Protocols). Formulations of the pharmaceutical compositions described herein may be prepared by methods known in the art of formulation. The preparatory methods may include bringing the antisense oligonucleotide into association with a diluent or another excipient and/or one or more other ingredients, and then if desirable, packaging (e.g. shaping) the product into a desired single- or multi-dose unit. The amount of the antisense oligonucleotide depends on the delivery approach and the specific formulation. The amount of the antisense oligonucleotide will also depend on the subject to be treated (size and condition) and also depend on route of administration. An antisense oligonucleotide, a conjugate or a pharmaceutical composition of the present invention is typically administered in an effective amount.

By way of example, the composition may comprise between 0.1% and 100% (w/w) of the antisense oligonucleotide.

The pharmaceutical formulations according to the present invention may also comprise one or more of the following: a pharmaceutically acceptable excipient, e.g. one or more solvents, dispersion media, diluents, liquid vehicles, dispersion or suspension aids, isotonic agents, surface active agents, preservatives, solid binders, thickening or emulsifying agents, lubricants and the like. It is of cause important that the added excipient are pharmaceutically acceptable and suited to the particular dosage form desired. Remington's The Science and Practice of Pharmacy, 21″Edition, A. R. Gennaro (Lippincott, Williams 8 Wilkins, Baltimore, Md., 2006; incorporated herein by reference) discloses various excipients used in formulating pharmaceutical compositions and known techniques for the preparation thereof.

In some embodiments, potential side effects from treatment with immune checkpoint inhibiting antisense oligonucleotides, such as breaking of immune self-tolerance, may be reduced or avoided by introducing means for target cell specific delivery, such as those described above for improving uptake or selective uptake of the antisense oligonucleotides in the target cells such as cancer cells, without the introduction of a general uptake increase in normal cells or in other tissues.

Thus, in some embodiments, the antisense oligonucleotide according to any one of the preceding claims, wherein the antisense oligonucleotide is conjugated with a ligand for targeted delivery. In some embodiments, the antisense oligonucleotide according to the invention is conjugated with folic acid or N-acetylgalactosamine (GalNAc). In some embodiments, the antisense oligonucleotide according to the invention is unconjugated. In some embodiments, the antisense oligonucleotide according to the invention is formulated in lipid nanoparticles for delivery to cells in vivo in a patient or to cells ex vivo.

When describing the embodiments of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The terms “comprising”, “comprise” and “comprises” herein are intended to be optionally substitutable with the terms “consisting of”, “consist of” and “consist of”, respectively, in every instance.

The invention will be more fully understood by reference to the following examples. They should not, however, be construed as limiting the scope of the invention. All literature citations are incorporated by reference.

### EXAMPLES

Example 1. LNA monomer and oligonucleotide synthesis may be performed using the methodology referred to in Examples 1 and 2 of WO2007/11275. Assessment of the stability of LNA oligonucleotides in human or rat plasma may be performed using the methodology referred to in Example 4 of WO2007/112754. Treatment of cultured cells with LNA-modified antisense oligonucleotides may be performed using the methodology referred to in Example 6 of WO2007/11275.

Example 2. RNA isolation and expression analysis from cultured cells and tissues is performed using the methodology referred to in Example 10 of WO2007/112754. RNAseq-based transcriptional profiling from cultured cells and tissues is performed using the methodology referred to in (Djebali et al. Nature 489: 101-108 or Chu et al. Nucleic Acid Ther. 22: 271-274 or Wang et al. Nature Reviews Genetics 10: 57-63).

Example 3. General Description of the Antisense Oligonucleotide Design Workflow.

Antisense oligonucleotides capable of decreasing the expression of target transcript(s) are designed as RNaseH-recruiting gapmer oligonucleotides. Gapmer oligonucleotides are designed by applying various locked nucleic acid (LNA)/DNA patterns (typically the patterns constitute a central region of DNA flanked by short LNA wings, e.g. LLLDDDDDDDDDDLLL, where L denotes LNA and D denotes DNA) to the reverse complement of target site sequences. Oligonucleotides that can bind to target sites with desired specificity in the transcriptome and have desired properties are synthesized and tested in vitro in cancer cell lines and subsequently in vivo in mouse tumour models. The ASOs of this invention, are listed in Table 3.1, 3.2, 5.1, 5.2, and 7.1 and 7.2 (LNA=uppercase, DNA lowercase, complete phosphorothioate backbone), and examples demonstrating their potential in knocking down PD1 (PDCD1) and CTLA-4 are described in example 5 below.

Example 4. Design of LNA-Modified Antisense Oligonucleotides for Knockdown of Multiple Targets.

LNA antisense oligonucleotides that can effectively knock down multiple targets listed in Table 1.1 and 1.2 were designed.

Table 1.1 and Table 1.2. List of targets comprise genes in antigen-presenting cells (APC)T cells and natural killer (NK) cells . The identity of the target genes and transcripts, and their corresponding mouse genes and transcripts are also described under “Terms and definitions” in the Detailed description above.

TABLE 1.1

| human | Target | | | |
|---|---|---|---|---|
| symbol | cell | alias | Ensembl gene id* | Ensembl transcript id* |
| CD274 | APC | PDL1 | ENSG00000120217 | ENST00000381577 |
| PDCD1LG2 | APC | PDL2 | ENSG00000197646 | ENST00000397747 |
| CD80 | APC | CD80 | ENSG00000121594 | ENST00000264246 |
| CD86 | APC | CD86 | ENSG00000114013 | ENST00000330540 |
| CD276 | APC | B7-H3 | ENSG00000103855 | ENST00000318443 |
| VTCN1 | APC | B7-H4 | ENSG00000134258 | ENST00000369458 |
| TNFRSF14 | APC | HVEM | ENSG00000157873 | ENST00000355716 |
| LGALS9 | APC | GAL9 | ENSG00000168961 | ENST00000395473 |
| IDO1 | APC | IDO1 | ENSG00000131203 | ENST00000518237 |
| HMOX1 | APC | HO1 | ENSG00000100292 | ENST00000216117 |
| PDCD1 | T cell | PD1 | ENSG00000188389 | ENST00000334409 |
| CTLA4 | T cell | CTLA4 | ENSG00000163599 | ENST00000302823 |

*Ensembl release 89

TABLE 1.2

| human | Target | | | |
|---|---|---|---|---|
| symbol | cell | alias | Ensembl gene id* | Ensembl transcript id* |
| LAG3 | T cell | LAG3 | ENSG00000089692 | ENST00000203629 |
| HAVCR2 | T cell | TIM3 | ENSG00000135077 | ENST00000307851 |
| TDO2 | APC | TDO | ENSG00000151790 | ENST00000536354 |
| TIGIT | T cell | TIGIT | ENSG00000181847 | ENST00000486257 |
| VSIR | APC | VISTA | ENSG00000107738 | ENST00000394957 |
| CEACAM1 | T cell | CECAM1 | ENSG00000079385 | ENST00000161559 |
| NT5E | APC | CD73 | ENSG00000135318 | ENST00000257770 |
| KIR2DL1 | NK cell | KIR2DL1 | ENSG00000125498 | ENST00000336077 |
| KIR2DL3 | NK cell | KIR2DL3 | ENSG00000243772 | ENST00000342376 |

*Ensembl release 89

In this example, the target sites (or target sequence in the Immune Checkpoint Protein encoding mRNAs) are shared by two or more targets in Table 1.1 and Table 1.2 and they have no more than ten predicted perfect match off-targets (Table 2.1: SEQ ID NOs: 1-361) (Table 2.2: SEQ ID Nos: 1653-1999). Additionally, target sites that are shared between two or more target transcripts by allowing for 1 mismatch are also considered (Table2.1: SEQ ID NOs: 362-376).

TABLE 2.1

| SEQ | | | |
|---|---|---|---|
| ID | target sequence | | |
| NO | (5′-3′) | targets | oligoID |
| 1 | AUCAGUCAUAAUCU | CD274\|IDO1 | |
| 2 | CAUUCUCCUGACCC | CD274\|PDCD1LG2 | |
| 3 | UCCAUGCCUUCUUUG | CD274\|PDCD1LG2 | |
| 4 | GAUAAAAAGUGUCA | CD276\|CD274 | |
| 5 | AGAGGAUAUGAAGC | CD276\|CD86 | |
| 6 | AGGAACUGAUCUUC | CD276\|CD86 | |
| 7 | CAGGCUCCUAGGAA | CD276\|CD86 | |
| 8 | GAGAGUUCUUCUCU | CD276\|CD86 | |
| 9 | GCCCAAGCCCUUCU | CD276\|CD86 | |
| 10 | GGCCCAAGCCCUUCU | CD276\|CD86 | |
| 11 | GGGCCCAAGCCCUU | CD276\|CD86 | |
| 12 | GGGCCCAAGCCCUUC | CD276\|CD86 | |
| 13 | GGGCCCAAGCCCUUCU | CD276\|CD86 | |
| 14 | UUGCCUCUGGCCAGC | CD276\|CD86 | |
| 15 | AAGGUUUAUAAUCC | CD276\|PDCD1LG2 | |
| 16 | GUAAGGUUUAUAAUC | CD276\|PDCD1LG2 | |
| 17 | GUAAGGUUUAUAAUCC | CD276\|PDCD1LG2 | |
| 18 | UAAGGUUUAUAAUC | CD276\|PDCD1LG2 | |
| 19 | UAAGGUUUAUAAUCC | CD276\|PDCD1LG2 | |
| 20 | CCCUCCCAGGACCUU | CD276\|TNFRSF14 | |
| 21 | CUGCAGCCUCUGAAA | CD276\|VTCN1 | |
| 22 | CUUCACUGGGGUUUU | CD276\|VTCN1 | |
| 23 | CUUCACUGGGGUUUUG | CD276\|VTCN1 | |
| 24 | GCUUCACUGGGGUU | CD276\|VTCN1 | |
| 25 | GCUUCACUGGGGUUU | CD276\|VTCN1 | |
| 26 | GCUUCACUGGGGUUUU | CD276\|VTCN1 | |
| 27 | GCUUCACUGGGGUUUUG | CD276\|VTCN1 | |
| 28 | GGCUUCACUGGGGU | CD276\|VTCN1 | |
| 29 | GGCUUCACUGGGGUU | CD276\|VTCN1 | |
| 30 | GGCUUCACUGGGGUUU | CD276\|VTCN1 | |
| 31 | GGCUUCACUGGGGUUUU | CD276\|VTCN1 | |
| 32 | GGCUUCACUGGGGUUUUG | CD276\|VTCN1 | |
| 33 | GGGUCAGGGAAAGAG | CD276\|VTCN1 | |
| 34 | UAGAAUCUGCUCCU | CD276\|VTCN1 | |
| 35 | UCCUUGACUGGGUA | CD276\|VTCN1 | |
| 36 | UUCACUGGGGUUUUG | CD276\|VTCN1 | |
| 37 | CAACCAGGUUUGAG | CD80\|CD274 | |
| 38 | UGACAUUCAUCUUC | CD80\|CD274 | |
| 39 | UGGGUAACUAAAUG | CD80\|CD274 | |
| 40 | UUGGGUAACUAAAU | CD80\|CD274 | |
| 41 | UUGGGUAACUAAAUG | CD80\|CD274 | |
| 42 | AACCAAGCAAGAGC | CD80\|CD86 | |
| 43 | AACCAAGCAAGAGCA | CD80\|CD86 | |
| 44 | ACCAAGCAAGAGCA | CD80\|CD86 | |
| 45 | AGUAACUGAUGAUG | CD80\|CD86 | |
| 46 | CACUUUGAGUUUCAG | CD80\|CD86 | |
| 47 | CACUUUGAGUUUCAGU | CD80\|CD86 | |
| 48 | CAGAUCACCUUAGA | CD80\|CD86 | |
| 49 | CCUCAGAAAAUUAAAAAUAG | CD80\|CD86 | |
| 50 | GAUGGAGAAAUGAAC | CD80\|CD86 | |
| 51 | GCUUUACCCAGGAG | CD80\|CD86 | |
| 52 | GGAUGGAGAAAUGAA | CD80\|CD86 | |
| 53 | GGAUGGAGAAAUGAAC | CD80\|CD86 | |
| 54 | GGCUUUACCCAGGA | CD80\|CD86 | |
| 55 | GGCUUUACCCAGGAG | CD80\|CD86 | |
| 56 | GUUCCUCAGAAAAUUAAAAA | CD80\|CD86 | |
| 57 | GUUCUGUUUGCCUCU | CD80\|CD86 | |
| 58 | UCAGAAAAUUAAAAAUAGAA | CD80\|CD86 | |
| 59 | UGUUCUGUUUGCCUC | CD80\|CD86 | |
| 60 | UGUUCUGUUUGCCUCU | CD80\|CD86 | |
| 61 | CUCUAAUCUAGCAG | CD80\|IDO1 | |
| 62 | AAAUCUCAGCUAAG | CD80\|PDCD1LG2 | |
| 63 | AGGUAUUUAAUUGG | CD80\|PDCD1LG2 | |
| 64 | CAGGUAUUUAAUUG | CD80\|PDCD1LG2 | |
| 65 | CAGGUAUUUAAUUGG | CD80\|PDCD1LG2 | |
| 66 | CUUUUGUAACCACC | CD80\|PDCD1LG2 | |
| 67 | UUAAAAAUACAAGAAAU | CD80\|PDCD1LG2 | |
| 68 | UUAAAAAUACAAGAAAUU | CD80\|PDCD1LG2 | |
| 69 | AAAGAGCCUCUCAA | CD80\|VTCN1 | |
| 70 | AAAGGAAGGAAAUCCUA | CD80\|VTCN1 | |
| 71 | AAAGGAAGGAAAUCCUAU | CD80\|VTCN1 | |
| 72 | AAAGGAAGGAAAUCCUAUC | CD80\|VTCN1 | |
| 73 | AAAGGAAGGAAAUCCUAUCA | CD80\|VTCN1 | |
| 74 | AAAUCCUAUCAUAUG | CD80\|VTCN1 | |
| 75 | AAAUCCUAUCAUAUGC | CD80\|VTCN1 | |
| 76 | AAAUCCUAUCAUAUGCU | CD80\|VTCN1 | |
| 77 | AAAUCCUAUCAUAUGCUA | CD80\|VTCN1 | |
| 78 | AAGGAAAUCCUAUCAUA | CD80\|VTCN1 | |
| 79 | AAGGAAAUCCUAUCAUAU | CD80\|VTCN1 | |
| 80 | AAGGAAAUCCUAUCAUAUG | CD80\|VTCN1 | |
| 81 | AAGGAAAUCCUAUCAUAUGC | CD80\|VTCN1 | |
| 82 | AAGGAAGGAAAUCCUA | CD80\|VTCN1 | |
| 83 | AAGGAAGGAAAUCCUAU | CD80\|VTCN1 | |
| 84 | AAGGAAGGAAAUCCUAUC | CD80\|VTCN1 | |
| 85 | AAGGAAGGAAAUCCUAUCA | CD80\|VTCN1 | |
| 86 | AAGGAAGGAAAUCCUAUCAU | CD80\|VTCN1 | |
| 87 | AAUCCUAUCAUAUGC | CD80\|VTCN1 | |
| 88 | AAUCCUAUCAUAUGCU | CD80\|VTCN1 | |
| 89 | AAUCCUAUCAUAUGCUA | CD80\|VTCN1 | |
| 90 | AGAGUUUCAGAUUUGCAAA | CD80\|VTCN1 | |
| 91 | AGAGUUUCAGAUUUGCAAAA | CD80\|VTCN1 | |
| 92 | AGAUUUGCAAAAUGAA | CD80\|VTCN1 | |
| 93 | AGAUUUGCAAAAUGAAA | CD80\|VTCN1 | |
| 94 | AGAUUUGCAAAAUGAAAA | CD80\|VTCN1 | |
| 95 | AGGAAAUCCUAUCAUA | CD80\|VTCN1 | |
| 96 | AGGAAAUCCUAUCAUAU | CD80\|VTCN1 | |
| 97 | AGGAAAUCCUAUCAUAUG | CD80\|VTCN1 | |
| 98 | AGGAAAUCCUAUCAUAUGC | CD80\|VTCN1 | |
| 99 | AGGAAAUCCUAUCAUAUGCU | CD80\|VTCN1 | |
| 100 | AGGAAGGAAAUCCUA | CD80\|VTCN1 | |
| 101 | AGGAAGGAAAUCCUAU | CD80\|VTCN1 | |
| 102 | AGGAAGGAAAUCCUAUC | CD80\|VTCN1 | |
| 103 | AGGAAGGAAAUCCUAUCA | CD80\|VTCN1 | |
| 104 | AGGAAGGAAAUCCUAUCAU | CD80\|VTCN1 | |
| 105 | AGGAAGGAAAUCCUAUCAUA | CD80\|VTCN1 | |
| 106 | AGUUUCAGAUUUGCAAA | CD80\|VTCN1 | |
| 107 | AGUUUCAGAUUUGCAAAA | CD80\|VTCN1 | |
| 108 | AGUUUCAGAUUUGCAAAAU | CD80\|VTCN1 | |
| 109 | AGUUUCAGAUUUGCAAAAUG | CD80\|VTCN1 | |
| 110 | AUAGAGUUUCAGAUUU | CD80\|VTCN1 | |
| 111 | AUAGAGUUUCAGAUUUG | CD80\|VTCN1 | |
| 112 | AUAGAGUUUCAGAUUUGC | CD80\|VTCN1 | |
| 113 | AUAGAGUUUCAGAUUUGCA | CD80\|VTCN1 | |
| 114 | AUAGAGUUUCAGAUUUGCAA | CD80\|VTCN1 | |
| 115 | AUCCUAUCAUAUGC | CD80\|VTCN1 | |
| 116 | AUCCUAUCAUAUGCU | CD80\|VTCN1 | |
| 117 | AUCCUAUCAUAUGCUA | CD80\|VTCN1 | |
| 118 | CAGAUUUGCAAAAUG | CD80\|VTCN1 | |
| 119 | CAGAUUUGCAAAAUGA | CD80\|VTCN1 | |
| 120 | CAGAUUUGCAAAAUGAA | CD80\|VTCN1 | |
| 121 | CAGAUUUGCAAAAUGAAA | CD80\|VTCN1 | |
| 122 | CAGAUUUGCAAAAUGAAAA | CD80\|VTCN1 | |
| 123 | CAGUGAACAAAGGAG | CD80\|VTCN1 | |
| 124 | CCUAUCAUAUGCUA | CD80\|VTCN1 | |
| 125 | CUAAGAAGCACCUA | CD80\|VTCN1 | |
| 126 | CUUUUUAAACAAACAA | CD80\|VTCN1 | |
| 127 | GAAAUCCUAUCAUAU | CD80\|VTCN1 | |
| 128 | GAAAUCCUAUCAUAUG | CD80\|VTCN1 | |
| 129 | GAAAUCCUAUCAUAUGC | CD80\|VTCN1 | |
| 130 | GAAAUCCUAUCAUAUGCU | CD80\|VTCN1 | |
| 131 | GAAAUCCUAUCAUAUGCUA | CD80\|VTCN1 | |
| 132 | GAAGGAAAUCCUAUCAU | CD80\|VTCN1 | |
| 133 | GAAGGAAAUCCUAUCAUA | CD80\|VTCN1 | |
| 134 | GAAGGAAAUCCUAUCAUAU | CD80\|VTCN1 | |
| 135 | GAAGGAAAUCCUAUCAUAUG | CD80\|VTCN1 | |
| 136 | GAGUUUCAGAUUUGCAAA | CD80\|VTCN1 | |
| 137 | GAGUUUCAGAUUUGCAAAA | CD80\|VTCN1 | |
| 138 | GAGUUUCAGAUUUGCAAAAU | CD80\|VTCN1 | |
| 139 | GAUUUGCAAAAUGAAA | CD80\|VTCN1 | |
| 140 | GAUUUGCAAAAUGAAAA | CD80\|VTCN1 | |
| 141 | GCAGUGAACAAAGGA | CD80\|VTCN1 | |
| 142 | GCAGUGAACAAAGGAG | CD80\|VTCN1 | |
| 143 | GGAAAUCCUAUCAUA | CD80\|VTCN1 | |
| 144 | GGAAAUCCUAUCAUAU | CD80\|VTCN1 | |
| 145 | GGAAAUCCUAUCAUAUG | CD80\|VTCN1 | |
| 146 | GGAAAUCCUAUCAUAUGC | CD80\|VTCN1 | |
| 147 | GGAAAUCCUAUCAUAUGCU | CD80\|VTCN1 | |
| 148 | GGAAAUCCUAUCAUAUGCUA | CD80\|VTCN1 | |
| 149 | GGAAGGAAAUCCUAU | CD80\|VTCN1 | |
| 150 | GGAAGGAAAUCCUAUC | CD80\|VTCN1 | |
| 151 | GGAAGGAAAUCCUAUCA | CD80\|VTCN1 | |
| 152 | GGAAGGAAAUCCUAUCAU | CD80\|VTCN1 | |
| 153 | GGAAGGAAAUCCUAUCAUA | CD80\|VTCN1 | |
| 154 | GGAAGGAAAUCCUAUCAUAU | CD80\|VTCN1 | |
| 155 | GUUUCAGAUUUGCAAA | CD80\|VTCN1 | |
| 156 | GUUUCAGAUUUGCAAAA | CD80\|VTCN1 | |
| 157 | GUUUCAGAUUUGCAAAAU | CD80\|VTCN1 | |
| 158 | GUUUCAGAUUUGCAAAAUG | CD80\|VTCN1 | |
| 159 | GUUUCAGAUUUGCAAAAUGA | CD80\|VTCN1 | |
| 160 | UAGAGUUUCAGAUUUG | CD80\|VTCN1 | |
| 161 | UAGAGUUUCAGAUUUGC | CD80\|VTCN1 | |
| 162 | UAGAGUUUCAGAUUUGCA | CD80\|VTCN1 | |
| 163 | UAGAGUUUCAGAUUUGCAA | CD80\|VTCN1 | |
| 164 | UAGAGUUUCAGAUUUGCAAA | CD80\|VTCN1 | |
| 165 | UCAGAUUUGCAAAAUG | CD80\|VTCN1 | |
| 166 | UCAGAUUUGCAAAAUGA | CD80\|VTCN1 | |
| 167 | UCAGAUUUGCAAAAUGAA | CD80\|VTCN1 | |
| 168 | UCAGAUUUGCAAAAUGAAA | CD80\|VTCN1 | |
| 169 | UCAGAUUUGCAAAAUGAAAA | CD80\|VTCN1 | |
| 170 | UCCUAUCAUAUGCU | CD80\|VTCN1 | |
| 171 | UCCUAUCAUAUGCUA | CD80\|VTCN1 | |
| 172 | UUCAGAUUUGCAAAA | CD80\|VTCN1 | |
| 173 | UUCAGAUUUGCAAAAU | CD80\|VTCN1 | |
| 174 | UUCAGAUUUGCAAAAUG | CD80\|VTCN1 | |
| 175 | UUCAGAUUUGCAAAAUGA | CD80\|VTCN1 | |
| 176 | UUCAGAUUUGCAAAAUGAA | CD80\|VTCN1 | |
| 177 | UUCAGAUUUGCAAAAUGAAA | CD80\|VTCN1 | |
| 178 | UUUCAGAUUUGCAAAA | CD80\|VTCN1 | |
| 179 | UUUCAGAUUUGCAAAAU | CD80\|VTCN1 | |
| 180 | UUUCAGAUUUGCAAAAUG | CD80\|VTCN1 | |
| 181 | UUUCAGAUUUGCAAAAUGA | CD80\|VTCN1 | |
| 182 | UUUCAGAUUUGCAAAAUGAA | CD80\|VTCN1 | |
| 183 | GUGUGAAUUACAGG | CD86\|CD274 | |
| 184 | GUUUUCCAUAAUUAG | CD86\|CD274 | |
| 185 | GUUUUCCAUAAUUAGG | CD86\|CD274 | |
| 186 | UGUGUGAAUUACAGG | CD86\|CD274 | |
| 187 | UGUUUUCCAUAAUUAG | CD86\|CD274 | |
| 188 | UGUUUUCCAUAAUUAGG | CD86\|CD274 | |
| 189 | UUUUCAUUUACAAAGA | CD86\|CD274 | |
| 190 | UUUUCCAUAAUUAGG | CD86\|CD274 | |
| 191 | AGUGGGAAGCCAAA | CD86\|IDO1 | |
| 192 | AGUGGGAAGCCAAAU | CD86\|IDO1 | |
| 193 | CAGUGGGAAGCCAAA | CD86\|IDO1 | |
| 194 | CAGUGGGAAGCCAAAU | CD86\|IDO1 | |
| 195 | GUGGGAAGCCAAAU | CD86\|IDO1 | |
| 196 | AAGAGAAGGAGAAGAGA | CD86\|LGALS9 | |
| 197 | CAGACAGUCAUCCA | CD86\|LGALS9 | |
| 198 | GAAGAGAAGGAGAAGAG | CD86\|LGALS9 | |
| 199 | GAAGAGAAGGAGAAGAGA | CD86\|LGALS9 | |
| 200 | CCAGUUCCAAACCC | CD86\|PDCD1 | |
| 201 | CUCUCAUCAACCCA | CD86\|PDCD1 | |
| 202 | CUCUCUCAUCAACC | CD86\|PDCD1 | |
| 203 | CUCUCUCAUCAACCC | CD86\|PDCD1 | |
| 204 | CUCUCUCAUCAACCCA | CD86\|PDCD1 | |
| 205 | GCCCAGGCCUGAGAC | CD86\|PDCD1 | |
| 206 | GGAGAUUCUGGGCA | CD86\|PDCD1 | |
| 207 | UCUCUCAUCAACCC | CD86\|PDCD1 | |
| 208 | UCUCUCAUCAACCCA | CD86\|PDCD1 | |
| 209 | AGACACUGGGAGAGG | CD86\|PDCD1LG2 | |
| 210 | CAACCUUCAGAAAG | CD86\|PDCD1LG2 | |
| 211 | CCAUGGAAGGGCCC | CD86\|PDCD1LG2 | |
| 212 | CUUCUUUGAGCCUCAGUUUC | CD86\|PDCD1LG2 | |
| 213 | CUUUUAUCUGCCCAG | CD86\|PDCD1LG2 | |
| 214 | GGAUGGAUGGAAAAA | CD86\|PDCD1LG2 | |
| 215 | UAGACACUGGGAGAG | CD86\|PDCD1LG2 | |
| 216 | UAGACACUGGGAGAGG | CD86\|PDCD1LG2 | |
| 217 | UUCUUAGCUCCUGA | CD86\|PDCD1LG2 | |
| 218 | AGCCGUCACCUCUU | CD86\|TNFRSF14 | |
| 219 | AGCCGUCACCUCUUG | CD86\|TNFRSF14 | |
| 220 | CAGCCGUCACCUCU | CD86\|TNFRSF14 | |
| 221 | CAGCCGUCACCUCUU | CD86\|TNFRSF14 | |
| 222 | CAGCCGUCACCUCUUG | CD86\|TNFRSF14 | |
| 223 | CCAGCCGUCACCUC | CD86\|TNFRSF14 | |
| 224 | CCAGCCGUCACCUCU | CD86\|TNFRSF14 | |
| 225 | CCAGCCGUCACCUCUU | CD86\|TNFRSF14 | |
| 226 | CCAGCCGUCACCUCUUG | CD86\|TNFRSF14 | |
| 227 | GCCGUCACCUCUUG | CD86\|TNFRSF14 | |
| 228 | UGAUGAAGCCCUGG | CD86\|TNFRSF14 | |
| 229 | CACACUACUGUGUA | CD86\|VTCN1 | |
| 230 | GAUGGGCAUGGCUC | CD86\|VTCN1 | |
| 231 | GAUGGGCAUGGCUCC | CD86\|VTCN1 | |
| 232 | GCAGUCCAAAGAUG | CD86\|VTCN1 | |
| 233 | GCCAGGAUAGAGAU | CD86\|VTCN1 | |
| 234 | GGAUGGGCAUGGCUC | CD86\|VTCN1 | |
| 235 | GGAUGGGCAUGGCUCC | CD86\|VTCN1 | |
| 236 | UCUUCUCCUAACUCU | CD86\|VTCN1 | |
| 237 | UUAGCAGCCAGAUC | CD86\|VTCN1 | |
| 238 | UUGGUUUACAAAUGC | CD86\|VTCN1 | |
| 239 | UUUUGCCAAGUCUC | CD86\|VTCN1 | |
| 240 | CUUCCGUAUUCCUC | CTLA4\|CD274 | |
| 241 | CUUCCGUAUUCCUCA | CTLA4\|CD274 | |
| 242 | GAAUUGGAUCAUGG | CTLA4\|CD274 | |
| 243 | UUCCGUAUUCCUCA | CTLA4\|CD274 | |
| 244 | AUCACAGGUGUUGG | CTLA4\|CD86 | |
| 245 | AUCACAGGUGUUGGU | CTLA4\|CD86 | |
| 246 | AUCACAGGUGUUGGUA | CTLA4\|CD86 | |
| 247 | CACAGGUGUUGGUA | CTLA4\|CD86 | |
| 248 | UCACAGGUGUUGGUA | CTLA4\|CD86 | |
| 249 | UAGAGCCCUAGAGU | CTLA4\|LGALS9 | |
| 250 | AAAAGAUGUUGUGUC | HMOX1\|CD80 | |
| 251 | AAAGAUGUUGUGUC | HMOX1\|CD80 | |
| 252 | UGUCCUGCUUCUAA | HMOX1\|CD80 | |
| 253 | UGUCCUGCUUCUAAA | HMOX1\|CD80 | |
| 254 | CCAGCCACAAGGCUG | HMOX1\|CD86 | |
| 255 | CAAGAGCAGGCAGGG | HMOX1\|PDCD1LG2 | |
| 256 | GCAAGAGCAGGCAGG | HMOX1\|PDCD1LG2 | |
| 257 | GCAAGAGCAGGCAGGG | HMOX1\|PDCD1LG2 | |
| 258 | GCAGGUGAGGGAACU | HMOX1\|PDCD1LG2 | |
| 259 | AACAUCAGCGUGGG | HMOX1\|VTCN1 | |
| 260 | UUGUUCAUUGGCUUA | PDCD1LG2\|IDO1 | |
| 261 | UCCUUCCUGGGUGGG | PDCD1\|LGALS9 | |
| 262 | CACCAGUGUUCUGC | PDCD1\|PDCD1LG2 | |
| 263 | GGAAAAGGGUUGAG | PDCD1\|PDCD1LG2 | |
| 264 | AGGCCCUUUGUGGG | PDCD1\|VTCN1 | |
| 265 | CAGGCCCUUUGUGG | PDCD1\|VTCN1 | |
| 266 | CAGGCCCUUUGUGGG | PDCD1\|VTCN1 | |
| 267 | CUCUGAAGCAUCUUU | PDCD1\|VTCN1 | |
| 268 | CUGUGAAGCGCUUG | TNFRSF14\|IDO1 | |
| 269 | ACAGGGAGCCUGCCC | TNFRSF14\|VTCN1 | |
| 270 | CUGGGGGCAGGGCCUG | TNFRSF14\|VTCN1 | |
| 271 | GGGCCAGCUCUGUGG | TNFRSF14\|VTCN1 | |
| 272 | GGGCCAGCUCUGUGGG | TNFRSF14\|VTCN1 | |
| 273 | AUCAAGUCCUGAGU | VTCN1\|CD274 | |
| 274 | AUCAAGUCCUGAGUG | VTCN1\|CD274 | |
| 275 | CAUCAAGUCCUGAGU | VTCN1\|CD274 | |
| 276 | CAUCAAGUCCUGAGUG | VTCN1\|CD274 | |
| 277 | CCAUCAAGUCCUGAG | VTCN1\|CD274 | |
| 278 | CCAUCAAGUCCUGAGU | VTCN1\|CD274 | |
| 279 | CCAUCAAGUCCUGAGUG | VTCN1\|CD274 | |
| 280 | UCAAGUCCUGAGUG | VTCN1\|CD274 | |
| 281 | CUCUUCUGAAAAUGC | VTCN1\|IDO1 | |
| 282 | CUCUUCUGAAAAUGCA | VTCN1\|IDO1 | |
| 283 | CUCUUCUGAAAAUGCAA | VTCN1\|IDO1 | |
| 284 | CUCUUCUGAAAAUGCAAA | VTCN1\|IDO1 | |
| 285 | CUUCUGAAAAUGCAA | VTCN1\|IDO1 | |
| 286 | CUUCUGAAAAUGCAAA | VTCN1\|IDO1 | |
| 287 | GUUUCCAGACAGGU | VTCN1\|IDO1 | |
| 288 | UCUUCUGAAAAUGCAA | VTCN1\|IDO1 | |
| 289 | UCUUCUGAAAAUGCAAA | VTCN1\|IDO1 | |
| 290 | UUCUCAUAGCCAUCC | VTCN1\|IDO1 | |
| 291 | ACUCCUGGGUGGCAG | VTCN1\|LGALS9 | |
| 292 | AGGCCAAUGAGGCA | VTCN1\|LGALS9 | |
| 293 | AGGCCAAUGAGGCAG | VTCN1\|LGALS9 | |
| 294 | AGGCCAAUGAGGCAGU | VTCN1\|LGALS9 | |
| 295 | CCAACAUCUAAAAG | VTCN1\|LGALS9 | |
| 296 | GCCAAUGAGGCAGU | VTCN1\|LGALS9 | |
| 297 | GGCCAAUGAGGCAGU | VTCN1\|LGALS9 | |
| 298 | AAACAAAAAGAAGCCA | VTCN1\|PDCD1LG2 | |
| 299 | AAGGUUUCCAGACAG | VTCN1\|PDCD1LG2 | |
| 300 | AAGGUUUCCAGACAGG | VTCN1\|PDCD1LG2 | |
| 301 | ACAUUCUGCCUCAGA | VTCN1\|PDCD1LG2 | |
| 302 | ACGUAUACACCAUA | VTCN1\|PDCD1LG2 | |
| 303 | ACGUAUACACCAUAG | VTCN1\|PDCD1LG2 | |
| 304 | ACGUAUACACCAUAGA | VTCN1\|PDCD1LG2 | |
| 305 | ACGUAUACACCAUAGAA | VTCN1\|PDCD1LG2 | |
| 306 | ACGUAUACACCAUAGAAU | VTCN1\|PDCD1LG2 | |
| 307 | ACGUAUACACCAUAGAAUA | VTCN1\|PDCD1LG2 | |
| 308 | ACGUAUACACCAUAGAAUAC | VTCN1\|PDCD1LG2 | |
| 309 | ACUGAUCUGGACUC | VTCN1\|PDCD1LG2 | |
| 310 | ACUGAUCUGGACUCA | VTCN1\|PDCD1LG2 | |
| 311 | AGAAAUAACUUCCUU | VTCN1\|PDCD1LG2 | |
| 312 | AGGAUUUCAAAAAUC | VTCN1\|PDCD1LG2 | |
| 313 | AGGGUCCACUGUUG | VTCN1\|PDCD1LG2 | |
| 314 | AGGUUUCCAGACAGG | VTCN1\|PDCD1LG2 | |
| 315 | AGUCCUCAGAGGCA | VTCN1\|PDCD1LG2 | |
| 316 | AUCCAAAACUACCC | VTCN1\|PDCD1LG2 | |
| 317 | CACGUAUACACCAUA | VTCN1\|PDCD1LG2 | |
| 318 | CACGUAUACACCAUAG | VTCN1\|PDCD1LG2 | |
| 319 | CACGUAUACACCAUAGA | VTCN1\|PDCD1LG2 | |
| 320 | CACGUAUACACCAUAGAA | VTCN1\|PDCD1LG2 | |
| 321 | CACGUAUACACCAUAGAAU | VTCN1\|PDCD1LG2 | |
| 322 | CACGUAUACACCAUAGAAUA | VTCN1\|PDCD1LG2 | |
| 323 | CAUUCUGCCUCAGA | VTCN1\|PDCD1LG2 | |
| 324 | CGUAUACACCAUAG | VTCN1\|PDCD1LG2 | |
| 325 | CGUAUACACCAUAGA | VTCN1\|PDCD1LG2 | |
| 326 | CGUAUACACCAUAGAA | VTCN1\|PDCD1LG2 | |
| 327 | CGUAUACACCAUAGAAU | VTCN1\|PDCD1LG2 | |
| 328 | CGUAUACACCAUAGAAUA | VTCN1\|PDCD1LG2 | |
| 329 | CGUAUACACCAUAGAAUAC | VTCN1\|PDCD1LG2 | |
| 330 | CGUAUACACCAUAGAAUACU | VTCN1\|PDCD1LG2 | |
| 331 | CUAAAGUGCAAUGC | VTCN1\|PDCD1LG2 | |
| 332 | CUGAUCUGGACUCA | VTCN1\|PDCD1LG2 | |
| 333 | GAUAACAUCUCUCA | VTCN1\|PDCD1LG2 | |
| 334 | GAUAACAUCUCUCAG | VTCN1\|PDCD1LG2 | |
| 335 | GCACGUAUACACCAUA | VTCN1\|PDCD1LG2 | |
| 336 | GCACGUAUACACCAUAG | VTCN1\|PDCD1LG2 | |
| 337 | GCACGUAUACACCAUAGA | VTCN1\|PDCD1LG2 | |
| 338 | GCACGUAUACACCAUAGAA | VTCN1\|PDCD1LG2 | |
| 339 | GCACGUAUACACCAUAGAAU | VTCN1\|PDCD1LG2 | |
| 340 | GGCACGUAUACACCAUA | VTCN1\|PDCD1LG2 | |
| 341 | GGCACGUAUACACCAUAG | VTCN1\|PDCD1LG2 | |
| 342 | GGCACGUAUACACCAUAGA | VTCN1\|PDCD1LG2 | |
| 343 | GGCACGUAUACACCAUAGAA | VTCN1\|PDCD1LG2 | |
| 344 | GUAUACACCAUAGAAUA | VTCN1\|PDCD1LG2 | |
| 345 | GUAUACACCAUAGAAUAC | VTCN1\|PDCD1LG2 | |
| 346 | GUAUACACCAUAGAAUACU | VTCN1\|PDCD1LG2 | |
| 347 | GUAUACACCAUAGAAUACUA | VTCN1\|PDCD1LG2 | |
| 348 | GUGGCACGUAUACACCAUA | VTCN1\|PDCD1LG2 | |
| 349 | GUGGCACGUAUACACCAUAG | VTCN1\|PDCD1LG2 | |
| 350 | UAACAAAUGCAUAGU | VTCN1\|PDCD1LG2 | |
| 351 | UAUGUUUUCUGAAUUU | VTCN1\|PDCD1LG2 | |
| 352 | UGGCACGUAUACACCAUA | VTCN1\|PDCD1LG2 | |
| 353 | UGGCACGUAUACACCAUAG | VTCN1\|PDCD1LG2 | |
| 354 | UGGCACGUAUACACCAUAGA | VTCN1\|PDCD1LG2 | |
| 355 | UGUAACACUCAGGU | VTCN1\|PDCD1LG2 | |
| 356 | UGUGGCACGUAUACACCAUA | VTCN1\|PDCD1LG2 | |
| 357 | UUAACAAAUGCAUAGU | VTCN1\|PDCD1LG2 | |
| 358 | UUUAACAAAUGCAUAG | VTCN1\|PDCD1LG2 | |
| 359 | UUUAACAAAUGCAUAGU | VTCN1\|PDCD1LG2 | |
| 360 | UUUUAACAAAUGCAUAG | VTCN1\|PDCD1LG2 | |
| 361 | UUUUAACAAAUGCAUAGU | VTCN1\|PDCD1LG2 | |
| 362 | GUACAAUUGCUCCAUUU | IDO1\|PDCD1LG2 | CRM0140 |
| 363 | CUUCCGUAUUCCUCAGU | CD274\|CTLA4 | CRM0141 |
| 364 | AAUUUUGUCGCCAAACU | CD274\|PDCD1LG2 | CRM0142 |
| 365 | ACCCUCUAGUGUUCCUG | PDCD1\|PDCD1LG2 | CRM0143 |
| 366 | CUUCCGUAUUCCUCAUG | CD274\|CTLA4 | CRM0144 |
| 367 | UUCACUUUCCCUGUAGG | CD274\|PDCD1LG2 | CRM0145 |
| 368 | AACAGUAUCUUAAGG | CD274\|IDO1\| | CRM0146 |
| | | PDCD1LG2 | |
| 369 | GGGCUGGACGUGCAG | IDO1\|PDCD1\| | CRM0147 |
| | | PDCD1LG2 | |
| 370 | CAACAAAAUCAACCAAAG | CD274\|PDCD1LG2 | CRM0148 |
| 371 | AGCUAGAUGCACUGUC | IDO1\|PDCD1LG2 | CRM0149 |
| 372 | UGACUUCCGUAUUCCUC | CD274\|CTLA4 | CRM0150 |
| 373 | CUCUCUCAUCAACCCAC | PDCD1\|PDCD1LG2 | CRM0151 |
| 374 | AACAUCUACCUCGCAGA | IDO1\|PDCD1LG2 | CRM0152 |
| 375 | GGCUUCCGUAUUCCUCA | CD274\|CTLA4 | CRM0153 |

TABLE 2.2

| SEQ | | | |
|---|---|---|---|
| ID | target sequence | | |
| NO | (5′-3′) | targets | oligoID |
| 1655 | AAAAAGAAAAGGAAAGGG | VSIR\|PDCD1LG2 | |
| 1656 | AAAAUCAAGGUGACAGC | HAVCR2\|KIR2DL1\| | |
| | | KIR2DL3 | |
| 1657 | AAAGCCCUCAGAAUC | KIR2DL3\|TIGIT | |
| 1658 | AAAGCCCUCAGAAUCC | KIR2DL3\|TIGIT | |
| 1659 | AAAGGAUGUAUCAGU | CD274\|VSIR | |
| 1660 | AAAGGAUGUAUCAGUU | CD274\|VSIR | |
| 1661 | AAAGUGAGUGAAGUG | VTCN1\|VSIR | |
| 1662 | AAAGUGAGUGAAGUGG | VTCN1\|VSIR | |
| 1663 | AACCUGCAGCAGGU | NT5E\|VTCN1 | |
| 1664 | AAGAAAACAACUCUG | NT5E\|PDCD1LG2 | |
| 1665 | AAGCCCUCAGAAUC | NT5E\|KIR2DL3\| | |
| | | TIGIT | |
| 1666 | AAGCCCUCAGAAUCC | KIR2DL3\|TIGIT | |
| 1667 | AAGGAAGAGGCUCUGC | PDCD1\|KIR2DL3 | |
| 1668 | AAGGAUGUAUCAGU | CD274\|VSIR | |
| 1669 | AAGGAUGUAUCAGUU | CD274\|VSIR | |
| 1670 | AAGGGGCAGAGGUGU | NT5E\|VSIR | |
| 1671 | AAGGGGCCCAGGACC | NT5E\|CD276 | |
| 1672 | AAGGGGCCCAGGACCA | NT5E\|CD276 | |
| 1673 | AAGGGGCCCAGGACCAC | NT5E\|CD276 | |
| 1674 | AAGUGAGUGAAGUGG | VTCN1\|VSIR | |
| 1675 | AAUAACAAAGAAUUAU | VTCN1\|TIGIT | |
| 1676 | AAUAUGUGUAAUGAAU | NT5E\|PDCD1LG2 | |
| 1677 | AAUCUUUUCCCUGGA | HAVCR2\|TDO2 | |
| 1678 | AAUCUUUUCCCUGGAA | HAVCR2\|TDO2 | |
| 1679 | AAUGAUAAAGUUAC | NT5E\|CD274 | |
| 1680 | AAUUCAUAAAAAUAC | VTCN1\|TDO2 | |
| 1681 | ACAUAGGAGCAUGG | NT5E\|CD276 | |
| 1682 | ACAUAGGAGCAUGGC | NT5E\|CD276 | |
| 1683 | ACAUAGGAGCAUGGCA | NT5E\|CD276 | |
| 1684 | ACCAGCAACUGAAG | PDCD1LG2\|TIGIT | |
| 1685 | ACCAGUCCAAGGCC | CD86\|TIGIT | |
| 1686 | AGAAAUGACUUUGAA | NT5E\|CD86 | |
| 1687 | AGAACAUGCAUUUUG | CEACAM1\|VSIR | |
| 1688 | AGAACAUGCAUUUUGG | CEACAM1\|VSIR | |
| 1689 | AGACACACGGAUGA | NT5E\|CD86 | |
| 1690 | AGACGGCACAGGCC | VSIR\|LGALS9 | |
| 1691 | AGACGGCACAGGCCA | VSIR\|LGALS9 | |
| 1692 | AGAGAAAAGGAAGAAAG | CEACAM1\|NT5E | |
| 1693 | AGAGAUGUCCAAGC | CD86\|VSIR | |
| 1694 | AGCAAAGAGAAGAUA | HAVCR2\|PDCD1LG2 | |
| 1695 | AGCCAUGGGUGUGAU | NT5E\|TNFRSF14 | |
| 1696 | AGCCAUGGGUGUGAUG | NT5E\|TNFRSF14 | |
| 1697 | AGCCAUGGGUGUGAUGA | NT5E\|TNFRSF14 | |
| 1698 | AGCCCACAGCCCAGA | VTCN1\|VSIR | |
| 1699 | AGCUCCUAUGACAU | KIR2DL1\|KIR2DL3\| | |
| | | TDO2 | |
| 1700 | AGCUCCUCACAGGCA | PDCD1LG2\|TDO2 | |
| 1701 | AGCUCCUCACAGGCAA | PDCD1LG2\|TDO2 | |
| 1702 | AGGAAAUCUGAUGCU | HAVCR2\|CD276 | |
| 1703 | AGGAUAAAAUUGGAU | NT5E\|CD86 | |
| 1704 | AGGAUGUAUCAGUU | CD274\|VSIR | |
| 1705 | AGGCAGAGCUGGAGGC | NT5E\|PDCD1 | |
| 1706 | AGGGGCCCAGGACCA | NT5E\|CD276 | |
| 1707 | AGGGGCCCAGGACCAC | NT5E\|CD276 | |
| 1708 | AGGGGGCUCCUGCC | LAG3\|CD276 | |
| 1709 | AGUGGGGUUACAUA | VTCN1\|VSIR | |
| 1710 | AGUGGGGUUACAUAA | VTCN1\|VSIR | |
| 1711 | AGUGGGGUUACAUAAC | VTCN1\|VSIR | |
| 1712 | AGUGGGGUUACAUAACU | VTCN1\|VSIR | |
| 1713 | AGUGGGGUUACAUAACUG | VTCN1\|VSIR | |
| 1714 | AGUGUAGUCACAGG | CD86\|KIR2DL1 | |
| 1715 | AGUUUGAAGUAUUCC | VTCN1\|TDO2 | |
| 1716 | AUAAACAAAAUAAUGUA | NT5E\|VTCN1 | |
| 1717 | AUAAAUGUUUGCCG | NT5E\|VTCN1 | |
| 1718 | AUAGGAGCAUGGCA | NT5E\|CD276 | |
| 1719 | AUAUGAUCAAUUGA | PDCD1LG2\|TDO2 | |
| 1720 | AUAUGAUCAAUUGAU | PDCD1LG2\|TDO2 | |
| 1721 | AUAUGUGUAAUGAAU | NT5E\|PDCD1LG2 | |
| 1722 | AUCCAAUAUACAAA | NT5E\|CD80 | |
| 1723 | AUCCUCUUGGCAUG | VTCN1\|TDO2 | |
| 1724 | AUCUUUUCCCUGGAA | HAVCR2\|TDO2 | |
| 1725 | AUGACCUCCAGGUUC | NT5E\|VTCN1 | |
| 1726 | AUGAGUAUGAGUAA | CD86\|TDO2 | |
| 1727 | AUGGAUAUAAGAUAU | CD86\|KIR2DL1\| | |
| | | KIR2DL3 | |
| 1728 | AUGUGGGGAGGGGGU | CEACAM1\|CD80 | |
| 1729 | AUGUGGGGAGGGGGUU | CEACAM1\|CD80 | |
| 1730 | AUUACCCAUUUCCCA | KIR2DL3\|CD274 | |
| 1731 | AUUACCCAUUUCCCAG | KIR2DL3\|CD274 | |
| 1732 | AUUCUCUAGAGAGU | VTCN1\|VSIR | |
| 1733 | AUUGUACAAGGAAAA | NT5E\|CD80 | |
| 1734 | AUUGUACAAGGAAAAU | NT5E\|CD80 | |
| 1735 | AUUGUACAAGGAAAAUU | NT5E\|CD80 | |
| 1736 | AUUGUACAAGGAAAAUUA | NT5E\|CD80 | |
| 1737 | AUUGUACAAGGAAAAUUAG | NT5E\|CD80 | |
| 1738 | AUUUGAGGCAAGAGA | KIR2DL1\|PDCD1LG2 | |
| 1739 | AUUUGUAAAUGUAUAU | HAVCR2\|VTCN1 | |
| 1740 | CAAAUGUCUAAGGU | CD80\|KIR2DL1\| | |
| | | KIR2DL3 | |
| 1741 | CAGAGCUGAGGUCAA | CEACAM1\|CD86 | |
| 1742 | CAGCCACAGAAAGAA | HAVCR2\|VTCN1 | |
| 1743 | CAGGGCUAGAUUGU | CEACAM1\|NT5E | |
| 1744 | CAUAAACAAAAUAAUG | NT5E\|VTCN1 | |
| 1745 | CAUAAACAAAAUAAUGU | NT5E\|VTCN1 | |
| 1746 | CAUAAACAAAAUAAUGUA | NT5E\|VTCN1 | |
| 1747 | CAUAGGAGCAUGGC | NT5E\|CD276 | |
| 1748 | CAUAGGAGCAUGGCA | NT5E\|CD276 | |
| 1749 | CAUGGGUGUGAUGA | NT5E\|TNFRSF14 | |
| 1750 | CCAAAAACAUUAAAA | NT5E\|HAVCR2 | |
| 1751 | CCAAGCCCUCAGAA | NT5E\|VSIR | |
| 1752 | CCACACCCACACACACC | CD86\|VSIR | |
| 1753 | CCAGGGCCCAAUAU | CEACAM1\|KIR2DL3 | |
| 1754 | CCAUGGGUGUGAUG | NT5E\|TNFRSF14 | |
| 1755 | CCAUGGGUGUGAUGA | NT5E\|TNFRSF14 | |
| 1756 | CCCAAAAACAUUAA | NT5E\|HAVCR2 | |
| 1757 | CCCAAAAACAUUAAA | NT5E\|HAVCR2 | |
| 1758 | CCCAAAAACAUUAAAA | NT5E\|HAVCR2 | |
| 1759 | CCCUUGGACACACA | NT5E\|TIGIT | |
| 1760 | CCGCGUCCAGCUGG | LAG3\|HMOX1 | |
| 1761 | CCUCUGAGUGGGUGG | NT5E\|CD276 | |
| 1762 | CCUGGUAGCAGCCU | CD80\|TIGIT | |
| 1763 | CGGAUGUGGGCACU | CEACAM1\|TNFRSF14 | |
| 1764 | CUCACCAAACACAA | CEACAM1\|CD86 | |
| 1765 | CUCACCAAACACAAG | CEACAM1\|CD86 | |
| 1766 | CUCAGAAAAUUAAAAAUAGA | CD80\|CD86 | |
| 1767 | CUCAGUGAGGCUGAC | CEACAM1\|VSIR | |
| 1768 | CUCCUCUGGUUGCU | NT5E\|CD86 | |
| 1769 | CUCCUGUCUGGCCCU | CD276\|VSIR | |
| 1770 | CUGACUCAGGGUGAG | CD86\|VSIR | |
| 1771 | CUGACUCAGGGUGAGA | CD86\|VSIR | |
| 1772 | CUGAGUCUGUUUCCUCAUC | CD274\|VSIR | |
| 1773 | CUGAGUCUGUUUCCUCAUCU | CD274\|VSIR | |
| 1774 | CUGCAGACAUUUGCUU | CEACAM1\|PDCD1LG2 | |
| 1775 | CUGCAUUAUCCUAU | CEACAM1\|CD276 | |
| 1776 | CUGCCAACACCAGCC | VSIR\|PDCD1LG2 | |
| 1777 | CUGCCAACACCAGCCA | VSIR\|PDCD1LG2 | |
| 1778 | CUGCCAACACCAGCCAC | VSIR\|PDCD1LG2 | |
| 1779 | CUGGGAAGUAGCAGA | CD80\|VSIR | |
| 1780 | CUGGGAAGUAGCAGAG | CD80\|VSIR | |
| 1781 | CUGGGAAGUAGCAGAGG | CD80\|VSIR | |
| 1782 | CUUCUUGCUUGGAGA | VSIR\|IDO1 | |
| 1783 | CUUGUUGGAAAGCA | NT5E\|LGALS9 | |
| 1784 | GAAAGCCCUCAGAAU | KIR2DL3\|TIGIT | |
| 1785 | GAAAGCCCUCAGAAUC | KIR2DL3\|TIGIT | |
| 1786 | GAAAGCCCUCAGAAUCC | KIR2DL3\|TIGIT | |
| 1787 | GAACAUGCAUUUUGG | CEACAM1\|VSIR | |
| 1788 | GAACCCUGGCCUUG | NT5E\|TIGIT | |
| 1789 | GAAGGAAGAGGCUCUG | PDCD1\|KIR2DL1\| | |
| | | KIR2DL3 | |
| 1790 | GAAGGAAGAGGCUCUGC | PDCD1\|KIR2DL3 | |
| 1791 | GAAUAUUCCUGUGG | NT5E\|CD80 | |
| 1792 | GACAUAGGAGCAUG | NT5E\|CD276 | |
| 1793 | GACAUAGGAGCAUGG | NT5E\|CD276 | |
| 1794 | GACAUAGGAGCAUGGC | NT5E\|CD276 | |
| 1795 | GACAUAGGAGCAUGGCA | NT5E\|CD276 | |
| 1796 | GACCCACAACACAG | HAVCR2\|PDCD1LG2 | |
| 1797 | GACGGCACAGGCCA | VSIR\|LGALS9 | |
| 1798 | GACUCAGGGUGAGA | CD86\|VSIR | |
| 1799 | GAGACAUGGCUGGUG | CD86\|TIGIT | |
| 1800 | GAGAGAAAAGGAAGAAAG | CEACAM1\|NT5E | |
| 1801 | GAGCAAGAACCGGA | LAG3\|CD80 | |
| 1802 | GAGGAUAAAAUUGGA | NT5E\|CD86 | |
| 1803 | GAGGAUAAAAUUGGAU | NT5E\|CD86 | |
| 1804 | GAGGCACUCUCAGG | CEACAM1\|VSIR | |
| 1805 | GAGGGGUAGAGGCC | NT5E\|VTCN1 | |
| 1806 | GAGUCUGUUUCCUCAUC | CD274\|VSIR | |
| 1807 | GAGUCUGUUUCCUCAUCU | CD274\|VSIR | |
| 1808 | GAUAGAUCUGAGGC | PDCD1LG2\|TIGIT | |
| 1809 | GAUCAUCAGUGAGU | NT5E\|VSIR | |
| 1810 | GAUGAGUAUGAGUA | CD86\|TDO2 | |
| 1811 | GAUGAGUAUGAGUAA | CD86\|TDO2 | |
| 1812 | GAUGGCCUGGGGAA | NT5E\|CD86 | |
| 1813 | GCAGGGCCCAGCAGGG | CD276\|TIGIT | |
| 1814 | GCAGUCUUUUCCUG | NT5E\|CD276 | |
| 1815 | GCAUAAACAAAAUAAU | NT5E\|VTCN1 | |
| 1816 | GCAUAAACAAAAUAAUG | NT5E\|VTCN1 | |
| 1817 | GCAUAAACAAAAUAAUGU | NT5E\|VTCN1 | |
| 1818 | GCAUAAACAAAAUAAUGUA | NT5E\|VTCN1 | |
| 1819 | GCAUCUAGUGCAGG | CEACAM1\|VSIR | |
| 1820 | GCCAACACCAGCCAC | VSIR\|PDCD1LG2 | |
| 1821 | GCCAGGAGGGCAAAG | NT5E\|LGALS9 | |
| 1822 | GCCAUGGGUGUGAU | NT5E\|TNFRSF14 | |
| 1823 | GCCAUGGGUGUGAUG | NT5E\|TNFRSF14 | |
| 1824 | GCCAUGGGUGUGAUGA | NT5E\|TNFRSF14 | |
| 1825 | GCCAUUUCAACCAU | VSIR\|PDCD1LG2 | |
| 1826 | GCUCCCUUAAUCCA | HAVCR2\|TIGIT | |
| 1827 | GCUCCCUUAAUCCAG | HAVCR2\|TIGIT | |
| 1828 | GCUCCUCACAGGCAA | PDCD1LG2\|TDO2 | |
| 1829 | GCUCCUUCUCUACCC | LAG3\|HAVCR2 | |
| 1830 | GCUCUUCUCCUCUCC | CD276\|TDO2 | |
| 1831 | GCUGCAUUAUCCUA | CEACAM1\|CD276 | |
| 1832 | GCUGCAUUAUCCUAU | CEACAM1\|CD276 | |
| 1833 | GCUGGGAAGUAGCAG | CD80\|VSIR | |
| 1834 | GCUGGGAAGUAGCAGA | CD80\|VSIR | |
| 1835 | GCUGGGAAGUAGCAGAG | CD80\|VSIR | |
| 1836 | GCUGGGAAGUAGCAGAGG | CD80\|VSIR | |
| 1837 | GCUUUGGCGUGGGA | NT5E\|PDCD1 | |
| 1838 | GGACCUGGGGUCAA | HMOX1\|VSIR | |
| 1839 | GGAGAAUGGUAGUG | CD276\|VSIR | |
| 1840 | GGAGUAAAUGUUUUU | CD276\|TDO2 | |
| 1841 | GGAGUCUCUUACUC | CD80\|KIR2DL1 | |
| 1842 | GGAUAAAAUUGGAU | NT5E\|CD86 | |
| 1843 | GGAUCCCUGGGGAAG | CEACAM1\|LAG3 | |
| 1844 | GGCAGUCUUUUCCUG | NT5E\|CD276 | |
| 1845 | GGCAUGAAAAUGGG | CD276\|VSIR | |
| 1846 | GGCAUGAAAAUGGGC | CD276\|VSIR | |
| 1847 | GGGAAGUAGCAGAGG | CD80\|VSIR | |
| 1848 | GGGACUCGGAGGGA | CD276\|VSIR | |
| 1849 | GGGAGGAGCUGGGGUC | TNFRSF14\|VSIR | |
| 1850 | GGGCAGUCUUUUCC | NT5E\|CD276 | |
| 1851 | GGGCAGUCUUUUCCU | NT5E\|CD276 | |
| 1852 | GGGCAGUCUUUUCCUG | NT5E\|CD276 | |
| 1853 | GGGGCCCAGGACCAC | NT5E\|CD276 | |
| 1854 | GGGGUUACAUAACU | VTCN1\|VSIR | |
| 1855 | GGGGUUACAUAACUG | VTCN1\|VSIR | |
| 1856 | GGGUUACAUAACUG | VTCN1\|VSIR | |
| 1857 | GGGUUCCUCUUUUUA | CEACAM1\|CD80 | |
| 1858 | GGUAAGAAUAUCAG | CEACAM1\|CD274 | |
| 1859 | GGUGCACACCCAGG | HMOX1\|NT5E | |
| 1860 | GGUUUCACAGCCUA | TIGIT\|TDO2 | |
| 1861 | GUACAAGGAAAAUUA | NT5E\|CD80 | |
| 1862 | GUACAAGGAAAAUUAG | NT5E\|CD80 | |
| 1863 | GUAGAAGUUAUGGA | CD86\|TDO2 | |
| 1864 | GUAGGCAGAAAAAUA | CD86\|TDO2 | |
| 1865 | GUAGGCAGAAAAAUAA | CD86\|TDO2 | |
| 1866 | GUAUAAAACAAACAC | LAG3\|PDCD1LG2 | |
| 1867 | GUAUGGCUAUGGCU | VTCN1\|TIGIT | |
| 1868 | GUCCCUACCAGGAA | CD276\|KIR2DL1 | |
| 1869 | GUCCCUACCAGGAAC | CD276\|KIR2DL1 | |
| 1870 | GUCCUCAGAGGCAU | NT5E\|PDCD1LG2 | |
| 1871 | GUCCUGGUAGCAGC | CD80\|TIGIT | |
| 1872 | GUCCUGGUAGCAGCC | CD80\|TIGIT | |
| 1873 | GUCCUGGUAGCAGCCU | CD80\|TIGIT | |
| 1874 | GUCUACCUGUAGGA | CEACAM1\|VTCN1 | |
| 1875 | GUCUACCUGUAGGAU | CEACAM1\|VTCN1 | |
| 1876 | GUCUACUUUGCAGC | CEACAM1\|LAG3 | |
| 1877 | GUCUAUGGUUGUAA | CD86\|TDO2 | |
| 1878 | GUCUCUGUUGCAACA | CD80\|TIGIT | |
| 1879 | GUCUCUGUUGCAACAA | CD80\|TIGIT | |
| 1880 | GUGAUAGAACCAGAA | NT5E\|TDO2 | |
| 1881 | GUGCCCAUGAAUUU | PDCD1LG2\|TDO2 | |
| 1882 | GUGGGCGGCCUGCU | LAG3\|PDCD1 | |
| 1883 | GUGGGCGGCCUGCUG | LAG3\|PDCD1 | |
| 1884 | GUGGGCGGCCUGCUGG | LAG3\|PDCD1 | |
| 1885 | GUGGGGUUACAUAA | VTCN1\|VSIR | |
| 1886 | GUGGGGUUACAUAAC | VTCN1\|VSIR | |
| 1887 | GUGGGGUUACAUAACU | VTCN1\|VSIR | |
| 1888 | GUGGGGUUACAUAACUG | VTCN1\|VSIR | |
| 1889 | GUGUCUGGUAUUGUU | NT5E\|CD274 | |
| 1890 | GUGUCUGUCUGUUCA | NT5E\|VSIR | |
| 1891 | GUUACAGCCUAUCU | NT5E\|CD276 | |
| 1892 | GUUACAGCCUAUCUC | NT5E\|CD276 | |
| 1893 | GUUCUAAUUUCAGCU | HAVCR2\|VTCN1 | |
| 1894 | GUUGGUCAUCAAAC | HAVCR2\|PDCD1LG2 | |
| 1895 | GUUUCAAGCCAGGG | VSIR\|LGALS9 | |
| 1896 | UAAAAUCAAGGUGAC | HAVCR2\|KIR2DL1\| | |
| | | KIR2DL3 | |
| 1897 | UAAAAUCAAGGUGACA | HAVCR2\|KIR2DL1\| | |
| | | KIR2DL3 | |
| 1898 | UAAAAUCAAGGUGACAG | HAVCR2\|KIR2DL1\| | |
| | | KIR2DL3 | |
| 1899 | UAAAAUCAAGGUGACAGC | HAVCR2\|KIR2DL1\| | |
| | | KIR2DL3 | |
| 1900 | UAAACAAAAUAAUGUA | NT5E\|VTCN1 | |
| 1901 | UAAAUCCUCUCCUC | NT5E\|PDCD1LG2 | |
| 1902 | UAACUUCCCUGUGUU | VTCN1\|VSIR | |
| 1903 | UAAGAAAACAACUCU | NT5E\|PDCD1LG2 | |
| 1904 | UAAGAAAACAACUCUG | NT5E\|PDCD1LG2 | |
| 1905 | UACAAGGAAAAUUAG | NT5E\|CD80 | |
| 1906 | UACCCAUUCAUAGU | NT5E\|CD86 | |
| 1907 | UAGAGAAAUCUCCC | NT5E\|CD86 | |
| 1908 | UAUCUAAGCUGCUU | VTCN1\|VSIR | |
| 1909 | UAUCUUCAUCUGUCC | VTCN1\|TIGIT | |
| 1910 | UAUUCUAAGUGGGU | TIGIT\|TDO2 | |
| 1911 | UCACCAAACACAAG | CEACAM1\|CD86 | |
| 1912 | UCACCAGCUACAGA | VSIR\|PDCD1LG2 | |
| 1913 | UCAGAAAAUUAAAAAUAGA | CD80\|CD86 | |
| 1914 | UCAGAUUGACCCUA | NT5E\|VTCN1 | |
| 1915 | UCCACACCCACACACA | CD86\|VSIR | |
| 1916 | UCCACACCCACACACAC | CD86\|VSIR | |
| 1917 | UCCACACCCACACACACC | CD86\|VSIR | |
| 1918 | UCCCUACCAGGAAC | CD276\|KIR2DL1 | |
| 1919 | UCCUGACCCUGCCCU | CD276\|VSIR | |
| 1920 | UCCUGGUAGCAGCC | CD80\|TIGIT | |
| 1921 | UCCUGGUAGCAGCCU | CD80\|TIGIT | |
| 1922 | UCCUGGUCUCUUCUA | HAVCR2\|TDO2 | |
| 1923 | UCUAAUCACCUCCA | NT5E\|VTCN1 | |
| 1924 | UCUACCUGUAGGAU | CEACAM1\|VTCN1 | |
| 1925 | UCUCAAGUUGGAUG | NT5E\|CD80 | |
| 1926 | UCUCACUUCAGUCC | VTCN1\|TIGIT | |
| 1927 | UCUCCAUCAGUCGC | KIR2DL1\|PDCD1LG2 | |
| 1928 | UCUCCUGUCUGGCCC | CD276\|VSIR | |
| 1929 | UCUCCUGUCUGGCCCU | CD276\|VSIR | |
| 1930 | UCUUCUAUUCUUUAG | VTCN1\|TDO2 | |
| 1931 | UCUUUUUCAGAAACUA | HAVCR2\|IDO1 | |
| 1932 | UGAAGCACACAGACA | NT5E\|LGALS9 | |
| 1933 | UGAAUAUUCCUGUGG | NT5E\|CD80 | |
| 1934 | UGAAUGCCUGCUCCA | CEACAM1\|CD276 | |
| 1935 | UGACUCAGGGUGAGA | CD86\|VSIR | |
| 1936 | UGAGUCUGUUUCCUCAUC | CD274\|VSIR | |
| 1937 | UGAGUCUGUUUCCUCAUCU | CD274\|VSIR | |
| 1938 | UGCAGACAUUUGCUU | CEACAM1\|PDCD1LG2 | |
| 1939 | UGCCAACACCAGCC | VSIR\|PDCD1LG2 | |
| 1940 | UGCCAACACCAGCCA | VSIR\|PDCD1LG2 | |
| 1941 | UGCCAACACCAGCCAC | VSIR\|PDCD1LG2 | |
| 1942 | UGCUGCAUUAUCCUA | CEACAM1\|CD276 | |
| 1943 | UGCUGCAUUAUCCUAU | CEACAM1\|CD276 | |
| 1944 | UGCUGGGCCCACAUU | KIR2DL1\|LGALS9 | |
| 1945 | UGGACUGAGCCUCAG | NT5E\|VSIR | |
| 1946 | UGGCAUGAAAAUGGG | CD276\|VSIR | |
| 1947 | UGGCAUGAAAAUGGGC | CD276\|VSIR | |
| 1948 | UGGGAAGUAGCAGAG | CD80\|VSIR | |
| 1949 | UGGGAAGUAGCAGAGG | CD80\|VSIR | |
| 1950 | UGGGCGGCCUGCUG | LAG3\|PDCD1 | |
| 1951 | UGGGCGGCCUGCUGG | LAG3\|PDCD1 | |
| 1952 | UGGGGUUACAUAAC | VTCN1\|VSIR | |
| 1953 | UGGGGUUACAUAACU | VTCN1\|VSIR | |
| 1954 | UGGGGUUACAUAACUG | VTCN1\|VSIR | |
| 1955 | UGGGUGGUGGGAAUA | VTCN1\|TDO2 | |
| 1956 | UGGGUUCCUCUUUUU | CEACAM1\|CD80 | |
| 1957 | UGGGUUCCUCUUUUUA | CEACAM1\|CD80 | |
| 1958 | UGUACAAGGAAAAUU | NT5E\|CD80 | |
| 1959 | UGUACAAGGAAAAUUA | NT5E\|CD80 | |
| 1960 | UGUACAAGGAAAAUUAG | NT5E\|CD80 | |
| 1961 | UGUAGGCAGAAAAAU | CD86\|TDO2 | |
| 1962 | UGUAGGCAGAAAAAUA | CD86\|TDO2 | |
| 1963 | UGUAGGCAGAAAAAUAA | CD86\|TDO2 | |
| 1964 | UGUAUGGCUAUGGC | VTCN1\|TIGIT | |
| 1965 | UGUAUGGCUAUGGCU | VTCN1\|TIGIT | |
| 1966 | UGUGUCUGUCUGUUCA | NT5E\|VSIR | |
| 1967 | UUACAGCCUAUCUC | NT5E\|CD276 | |
| 1968 | UUCCUCACCUCUCUCC | PDCD1\|KIR2DL1\| | |
| | | KIR2DL3 | |
| 1969 | UUCCUCAGAAAAUUAAAAAU | CD80\|CD86 | |
| 1970 | UUCUCACUUCAGUCC | VTCN1\|TIGIT | |
| 1971 | UUCUUCUAUUCUUUAG | VTCN1\|TDO2 | |
| 1972 | UUGCAAGGGUGCCA | VSIR\|PDCD1LG2 | |
| 1973 | UUGCUGCAUUAUCCU | CEACAM1\|CD276 | |
| 1974 | UUGCUGCAUUAUCCUA | CEACAM1\|CD276 | |
| 1975 | UUGCUGCAUUAUCCUAU | CEACAM1\|CD276 | |
| 1976 | UUGGACUGAGCCUC | NT5E\|VSIR | |
| 1977 | UUGGACUGAGCCUCA | NT5E\|VSIR | |
| 1978 | UUGGACUGAGCCUCAG | NT5E\|VSIR | |
| 1979 | UUGGGUUCCUCUUUU | CEACAM1\|CD80 | |
| 1980 | UUGGGUUCCUCUUUUU | CEACAM1\|CD80 | |
| 1981 | UUGGGUUCCUCUUUUUA | CEACAM1\|CD80 | |
| 1982 | UUGUACAAGGAAAAU | NT5E\|CD80 | |
| 1983 | UUGUACAAGGAAAAUU | NT5E\|CD80 | |
| 1984 | UUGUACAAGGAAAAUUA | NT5E\|CD80 | |
| 1985 | UUGUACAAGGAAAAUUAG | NT5E\|CD80 | |
| 1986 | UUUCUUCUAUUCUUUA | VTCN1\|TDO2 | |
| 1987 | UUUCUUCUAUUCUUUAG | VTCN1\|TDO2 | |
| 1988 | UUUGGACUGAGCCUC | NT5E\|VSIR | |
| 1989 | UUUGGACUGAGCCUCA | NT5E\|VSIR | |
| 1990 | UUUGGACUGAGCCUCAG | NT5E\|VSIR | |
| 1991 | UUUGGGUUCCUCUUU | CEACAM1\|CD80 | |
| 1992 | UUUGGGUUCCUCUUUU | CEACAM1\|CD80 | |
| 1993 | UUUGGGUUCCUCUUUUU | CEACAM1\|CD80 | |
| 1994 | UUUGGGUUCCUCUUUUUA | CEACAM1\|CD80 | |
| 1995 | UUUUCUUCUAUUCUUUA | VTCN1\|TDO2 | |
| 1996 | UUUUCUUCUAUUCUUUAG | VTCN1\|TDO2 | |
| 1997 | UUUUGGGUUCCUCUU | CEACAM1\|CD80 | |
| 1998 | UUUUGGGUUCCUCUUU | CEACAM1\|CD80 | |
| 1999 | UUUUGGGUUCCUCUUUU | CEACAM1\|CD80 | |
| 2000 | UUUUGGGUUCCUCUUUUU | CEACAM1\|CD80 | |
| 2001 | UUUUGGGUUCCUCUUUUUA | CEACAM1\|CD80 | |

LNA-modified ASOs were designed against each of the target sites listed above in Table 2.1 and Table 2.2 (see below in Table 3.1: SEQ ID NOs: 376-1475; and Table 3.2: SEQ ID NOs: 2002-3043; LNA shown in uppercase, DNA lowercase).

TABLE 3.1

| SEQ | Oligonucleotide | | |
|---|---|---|---|
| ID NO | (5′-3′) | targets | oligoID |
| 376 | AGATtatgactGAT | CD274\|IDO1 | |
| 377 | AGAttatgacTGAT | CD274\|IDO1 | CRM0193 |
| 378 | AGATtatgacTGAT | CD274\|IDO1 | |
| 379 | GGGTcaggagaATG | CD274\|PDCD1LG2 | |
| 380 | GGGtcaggagaaTG | CD274\|PDCD1LG2 | |
| 381 | GGGTcaggagAATG | CD274\|PDCD1LG2 | |
| 382 | CAAagaaggcaTGGA | CD274\|PDCD1LG2 | CRM0196 |
| 383 | CAAAgaaggcaTGGA | CD274\|PDCD1LG2 | |
| 384 | CAAAgaaggcatGGA | CD274\|PDCD1LG2 | |
| 385 | TGAcacttttTATC | CD276\|CD274 | |
| 386 | TGACacttttTATC | CD276\|CD274 | |
| 387 | TGACactttttATC | CD276\|CD274 | |
| 388 | GCTTcatatcCTCT | CD276\|CD86 | |
| 389 | GCttcatatcctCT | CD276\|CD86 | |
| 390 | GCTtcatatccTCT | CD276\|CD86 | |
| 391 | GAagatcagttCCT | CD276\|CD86 | |
| 392 | GAAGatcagtTCCT | CD276\|CD86 | |
| 393 | GAAGatcagttCCT | CD276\|CD86 | |
| 394 | TTcctaggagccTG | CD276\|CD86 | |
| 395 | TTCctaggagcCTG | CD276\|CD86 | |
| 396 | TTCctaggagCCTG | CD276\|CD86 | |
| 397 | AGAgaagaacTCTC | CD276\|CD86 | |
| 398 | AGAGaagaactCTC | CD276\|CD86 | |
| 399 | AGAGaagaacTCTC | CD276\|CD86 | |
| 400 | AGAAgggcttgGGC | CD276\|CD86 | |
| 401 | AGAagggcttggGC | CD276\|CD86 | |
| 402 | AGaagggcttggGC | CD276\|CD86 | |
| 403 | AGaagggcttggGCC | CD276\|CD86 | |
| 404 | AGAagggcttgggCC | CD276\|CD86 | |
| 405 | AGaagggcttgggCC | CD276\|CD86 | |
| 406 | AAGggcttgggcCC | CD276\|CD86 | |
| 407 | AAgggcttgggcCC | CD276\|CD86 | |
| 408 | AAgggcttgggCCC | CD276\|CD86 | |
| 409 | GAagggcttgggcCC | CD276\|CD86 | |
| 410 | GAAgggcttgggcCC | CD276\|CD86 | |
| 411 | GAagggcttgggCCC | CD276\|CD86 | |
| 412 | AGAagggcttgggcCC | CD276\|CD86 | |
| 413 | AGaagggcttgggCCC | CD276\|CD86 | |
| 414 | AGaagggcttgggcCC | CD276\|CD86 | |
| 415 | GCtggccagaggCAA | CD276\|CD86 | |
| 416 | GCtggccagaggcAA | CD276\|CD86 | |
| 417 | GCTggccagaggcAA | CD276\|CD86 | |
| 418 | GGATtataaaCCTT | CD276\|PDCD1LG2 | |
| 419 | GGAttataaaCCTT | CD276\|PDCD1LG2 | |
| 420 | GGATtataaacCTT | CD276\|PDCD1LG2 | |
| 421 | GATTataaacctTAC | CD276\|PDCD1LG2 | |
| 422 | GATtataaaccTTAC | CD276\|PDCD1LG2 | |
| 423 | GATTataaaccTTAC | CD276\|PDCD1LG2 | |
| 424 | GGATtataaaccTTAC | CD276\|PDCD1LG2 | |
| 425 | GGATtataaacctTAC | CD276\|PDCD1LG2 | |
| 426 | GGAttataaaccTTAC | CD276\|PDCD1LG2 | |
| 427 | GATtataaacCTTA | CD276\|PDCD1LG2 | |
| 428 | GATTataaaccTTA | CD276\|PDCD1LG2 | |
| 429 | GATTataaacCTTA | CD276\|PDCD1LG2 | |
| 430 | GGAttataaacCTTA | CD276\|PDCD1LG2 | |
| 431 | GGATtataaaccTTA | CD276\|PDCD1LG2 | |
| 432 | GGATtataaacCTTA | CD276\|PDCD1LG2 | |
| 433 | AAggtcctgggAGGG | CD276\|TNFRSF14 | |
| 434 | AAGgtcctgggagGG | CD276\|TNFRSF14 | |
| 435 | AAggtcctgggagGG | CD276\|TNFRSF14 | |
| 436 | TTTCagaggctGCAG | CD276\|VTCN1 | |
| 437 | TTTcagaggctgcAG | CD276\|VTCN1 | |
| 438 | TTtcagaggctGCAG | CD276\|VTCN1 | |
| 439 | AAAaccccagtGAAG | CD276\|VTCN1 | |
| 440 | AAAAccccagtGAAG | CD276\|VTCN1 | |
| 441 | AAaaccccagtGAAG | CD276\|VTCN1 | |
| 442 | CAAaaccccagtgaAG | CD276\|VTCN1 | |
| 443 | CAAaaccccagtGAAG | CD276\|VTCN1 | |
| 444 | CAAAaccccagtGAAG | CD276\|VTCN1 | |
| 445 | AACCccagtgAAGC | CD276\|VTCN1 | |
| 446 | AAccccagtgaaGC | CD276\|VTCN1 | |
| 447 | AACcccagtgaAGC | CD276\|VTCN1 | |
| 448 | AAACcccagtgAAGC | CD276\|VTCN1 | |
| 449 | AAAccccagtgAAGC | CD276\|VTCN1 | |
| 450 | AAaccccagtgaaGC | CD276\|VTCN1 | |
| 451 | AAAAccccagtgaAGC | CD276\|VTCN1 | |
| 452 | AAAAccccagtgAAGC | CD276\|VTCN1 | |
| 453 | AAaaccccagtgaaGC | CD276\|VTCN1 | |
| 454 | CAaaaccccagtgaaGC | CD276\|VTCN1 | |
| 455 | CAAaaccccagtgaaGC | CD276\|VTCN1 | |
| 456 | CAAaaccccagtgAAGC | CD276\|VTCN1 | |
| 457 | ACCccagtgaagCC | CD276\|VTCN1 | |
| 458 | ACcccagtgaagCC | CD276\|VTCN1 | |
| 459 | ACcccagtgaaGCC | CD276\|VTCN1 | |
| 460 | AAccccagtgaagCC | CD276\|VTCN1 | |
| 461 | AACCccagtgaagCC | CD276\|VTCN1 | |
| 462 | AACcccagtgaagCC | CD276\|VTCN1 | |
| 463 | AAAccccagtgaaGCC | CD276\|VTCN1 | |
| 464 | AAAccccagtgaagCC | CD276\|VTCN1 | |
| 465 | AAaccccagtgaagCC | CD276\|VTCN1 | |
| 466 | AAAaccccagtgaagCC | CD276\|VTCN1 | |
| 467 | AAaaccccagtgaaGCC | CD276\|VTCN1 | |
| 468 | AAaaccccagtgaagCC | CD276\|VTCN1 | |
| 469 | CAAAaccccagtgaagCC | CD276\|VTCN1 | |
| 470 | CAaaaccccagtgaagCC | CD276\|VTCN1 | |
| 471 | CAAaaccccagtgaagCC | CD276\|VTCN1 | |
| 472 | CTCtttccctgacCC | CD276\|VTCN1 | |
| 473 | CTctttccctgaCCC | CD276\|VTCN1 | |
| 474 | CTctttccctgacCC | CD276\|VTCN1 | |
| 475 | AGGAgcagattCTA | CD276\|VTCN1 | |
| 476 | AGGAgcagatTCTA | CD276\|VTCN1 | |
| 477 | AGgagcagattCTA | CD276\|VTCN1 | |
| 478 | TAcccagtcaAGGA | CD276\|VTCN1 | |
| 479 | TACCcagtcaAGGA | CD276\|VTCN1 | |
| 480 | TAcccagtcaagGA | CD276\|VTCN1 | |
| 481 | CAAaaccccagTGAA | CD276\|VTCN1 | |
| 482 | CAAAaccccagtGAA | CD276\|VTCN1 | |
| 483 | CAAAaccccagTGAA | CD276\|VTCN1 | |
| 484 | CTCaaacctgGTTG | CD80\|CD274 | |
| 485 | CTCAaacctgGTTG | CD80\|CD274 | |
| 486 | CTCAaacctggtTG | CD80\|CD274 | |
| 487 | GAAGatgaatGTCA | CD80\|CD274 | |
| 488 | GAAgatgaatGTCA | CD80\|CD274 | |
| 489 | GAAGatgaatgTCA | CD80\|CD274 | |
| 490 | CAtttagttacCCA | CD80\|CD274 | |
| 491 | CAtttagttaCCCA | CD80\|CD274 | |
| 492 | CATTtagttaCCCA | CD80\|CD274 | |
| 493 | ATTTagttacCCAA | CD80\|CD274 | |
| 494 | ATTtagttacCCAA | CD80\|CD274 | |
| 495 | ATttagttacCCAA | CD80\|CD274 | |
| 496 | CATttagttacCCAA | CD80\|CD274 | |
| 497 | CATttagttaccCAA | CD80\|CD274 | |
| 498 | CATTtagttacCCAA | CD80\|CD274 | |
| 499 | GCtcttgcttgGTT | CD80\|CD86 | |
| 500 | GCtcttgcttggTT | CD80\|CD86 | |
| 501 | GCTCttgcttgGTT | CD80\|CD86 | |
| 502 | TGCtcttgcttgGTT | CD80\|CD86 | |
| 503 | TGctcttgcttggTT | CD80\|CD86 | |
| 504 | TGctcttgcttgGTT | CD80\|CD86 | |
| 505 | TGctcttgcttgGT | CD80\|CD86 | |
| 506 | TGctcttgcttGGT | CD80\|CD86 | |
| 507 | TGCTcttgcttGGT | CD80\|CD86 | |
| 508 | CATCatcagtTACT | CD80\|CD86 | |
| 509 | CATcatcagtTACT | CD80\|CD86 | |
| 510 | CATCatcagttACT | CD80\|CD86 | |
| 511 | CTGAaactcaaaGTG | CD80\|CD86 | |
| 512 | CTGaaactcaaAGTG | CD80\|CD86 | |
| 513 | CTGAaactcaaAGTG | CD80\|CD86 | |
| 514 | ACTGaaactcaaAGTG | CD80\|CD86 | |
| 515 | ACTGaaactcaaaGTG | CD80\|CD86 | |
| 516 | ACTgaaactcaaAGTG | CD80\|CD86 | |
| 517 | TCTAaggtgaTCTG | CD80\|CD86 | |
| 518 | TCTaaggtgaTCTG | CD80\|CD86 | |
| 519 | TCTAaggtgatCTG | CD80\|CD86 | |
| 520 | CTatttttaattttctgaGG | CD80\|CD86 | |
| 521 | CTatttttaattttctgAGG | CD80\|CD86 | |
| 522 | CTATttttaattttctGAGG | CD80\|CD86 | |
| 523 | GTTcatttctccaTC | CD80\|CD86 | |
| 524 | GTTCatttctcCATC | CD80\|CD86 | |
| 525 | GTTcatttctcCATC | CD80\|CD86 | |
| 526 | CTCCtgggtaAAGC | CD80\|CD86 | |
| 527 | CTcctgggtaaaGC | CD80\|CD86 | |
| 528 | CTCctgggtaaAGC | CD80\|CD86 | |
| 529 | TTCAtttctccATCC | CD80\|CD86 | |
| 530 | TTCAtttctccatCC | CD80\|CD86 | |
| 531 | TTcatttctccatCC | CD80\|CD86 | |
| 532 | GTTcatttctccATCC | CD80\|CD86 | |
| 533 | GTTcatttctccatCC | CD80\|CD86 | |
| 534 | GTtcatttctccatCC | CD80\|CD86 | |
| 535 | TCctgggtaaaGCC | CD80\|CD86 | |
| 536 | TCctgggtaaaGCC | CD80\|CD86 | |
| 537 | TCctgggtaaagCC | CD80\|CD86 | |
| 538 | CTCctgggtaaagCC | CD80\|CD86 | |
| 539 | CTcctgggtaaaGCC | CD80\|CD86 | |
| 540 | CTcctgggtaaagCC | CD80\|CD86 | |
| 541 | TTTTtaattttctgagGAAC | CD80\|CD86 | |
| 542 | TTTTtaattttctgaggaAC | CD80\|CD86 | |
| 543 | TTTttaattttctgagGAAC | CD80\|CD86 | |
| 544 | AGAGgcaaacagAAC | CD80\|CD86 | |
| 545 | AGAGgcaaacaGAAC | CD80\|CD86 | |
| 546 | AGAggcaaacaGAAC | CD80\|CD86 | |
| 547 | TTCtatttttaattttctGA | CD80\|CD86 | |
| 548 | TTCTatttttaattttCTGA | CD80\|CD86 | |
| 549 | TTCTatttttaattttcTGA | CD80\|CD86 | |
| 550 | GAGgcaaacagaACA | CD80\|CD86 | |
| 551 | GAGGcaaacagaACA | CD80\|CD86 | |
| 552 | GAGGcaaacagAACA | CD80\|CD86 | |
| 553 | AGAggcaaacagaaCA | CD80\|CD86 | |
| 554 | AGAGgcaaacagAACA | CD80\|CD86 | |
| 555 | AGAGgcaaacagaACA | CD80\|CD86 | |
| 556 | CTGCtagattagAG | CD80\|IDO1 | |
| 557 | CTGCtagattaGAG | CD80\|IDO1 | |
| 558 | CTGCtagattAGAG | CD80\|IDO1 | |
| 559 | CTTAgctgagaTTT | CD80\|PDCD1LG2 | |
| 560 | CTTAgctgagATTT | CD80\|PDCD1LG2 | |
| 561 | CTTagctgagATTT | CD80\|PDCD1LG2 | |
| 562 | CCAAttaaataCCT | CD80\|PDCD1LG2 | |
| 563 | CCAAttaaatACCT | CD80\|PDCD1LG2 | |
| 564 | CCAattaaatACCT | CD80\|PDCD1LG2 | |
| 565 | CAATtaaataCCTG | CD80\|PDCD1LG2 | |
| 566 | CAattaaataCCTG | CD80\|PDCD1LG2 | |
| 567 | CAAttaaataCCTG | CD80\|PDCD1LG2 | |
| 568 | CCAAttaaataCCTG | CD80\|PDCD1LG2 | |
| 569 | CCAattaaataCCTG | CD80\|PDCD1LG2 | |
| 570 | CCaattaaataCCTG | CD80\|PDCD1LG2 | |
| 571 | GGTGgttacaaaAG | CD80\|PDCD1LG2 | |
| 572 | GGTGgttacaaAAG | CD80\|PDCD1LG2 | |
| 573 | GGTGgttacaAAAG | CD80\|PDCD1LG2 | |
| 574 | ATTtcttgtatttTTAA | CD80\|PDCD1LG2 | |
| 575 | ATTTcttgtatttTTAA | CD80\|PDCD1LG2 | |
| 576 | ATTTcttgtattttTAA | CD80\|PDCD1LG2 | |
| 577 | AATttcttgtatttTTAA | CD80\|PDCD1LG2 | |
| 578 | AATTtcttgtattttTAA | CD80\|PDCD1LG2 | |
| 579 | AATTtcttgtatttTTAA | CD80\|PDCD1LG2 | |
| 580 | TTGAgaggctCTTT | CD80\|VTCN1 | |
| 581 | TTGAgaggctctTT | CD80\|VTCN1 | |
| 582 | TTGAgaggctcTTT | CD80\|VTCN1 | |
| 583 | TAggatttccttccTTT | CD80\|VTCN1 | |
| 584 | TAggatttccttcctTT | CD80\|VTCN1 | |
| 585 | TAGGatttccttccTTT | CD80\|VTCN1 | |
| 586 | ATaggatttccttccTTT | CD80\|VTCN1 | |
| 587 | ATAGgatttccttcctTT | CD80\|VTCN1 | |
| 588 | ATaggatttccttcctTT | CD80\|VTCN1 | |
| 589 | GAtaggatttccttccTTT | CD80\|VTCN1 | |
| 590 | GAtaggatttccttcctTT | CD80\|VTCN1 | |
| 591 | GATaggatttccttcctTT | CD80\|VTCN1 | |
| 592 | TGAtaggatttccttcctTT | CD80\|VTCN1 | |
| 593 | TGataggatttccttcctTT | CD80\|VTCN1 | |
| 594 | TGataggatttccttccTTT | CD80\|VTCN1 | |
| 595 | CATAtgataggaTTT | CD80\|VTCN1 | |
| 596 | CATAtgataggATTT | CD80\|VTCN1 | |
| 597 | CATatgataggATTT | CD80\|VTCN1 | |
| 598 | GCATatgataggATTT | CD80\|VTCN1 | |
| 599 | GCAtatgataggaTTT | CD80\|VTCN1 | |
| 600 | GCATatgataggaTTT | CD80\|VTCN1 | |
| 601 | AGCatatgataggatTT | CD80\|VTCN1 | |
| 602 | AGCAtatgataggATTT | CD80\|VTCN1 | |
| 603 | AGCAtatgataggaTTT | CD80\|VTCN1 | |
| 604 | TAGCatatgataggaTTT | CD80\|VTCN1 | |
| 605 | TAGCatatgataggatTT | CD80\|VTCN1 | |
| 606 | TAGCatatgataggATTT | CD80\|VTCN1 | |
| 607 | TAtgataggatttcCTT | CD80\|VTCN1 | |
| 608 | TATGataggatttcCTT | CD80\|VTCN1 | |
| 609 | TATGataggatttCCTT | CD80\|VTCN1 | |
| 610 | ATatgataggatttCCTT | CD80\|VTCN1 | |
| 611 | ATAtgataggatttccTT | CD80\|VTCN1 | |
| 612 | ATATgataggatttCCTT | CD80\|VTCN1 | |
| 613 | CATAtgataggatttcCTT | CD80\|VTCN1 | |
| 614 | CATatgataggatttccTT | CD80\|VTCN1 | |
| 615 | CAtatgataggatttccTT | CD80\|VTCN1 | |
| 616 | GCatatgataggatttccTT | CD80\|VTCN1 | |
| 617 | GCAtatgataggatttccTT | CD80\|VTCN1 | |
| 618 | GCatatgataggatttccTT | CD80\|VTCN1 | |
| 619 | TAGgatttccttccTT | CD80\|VTCN1 | |
| 620 | TAGGatttccttcCTT | CD80\|VTCN1 | |
| 621 | TAggatttccttccTT | CD80\|VTCN1 | |
| 622 | ATaggatttccttCCTT | CD80\|VTCN1 | |
| 623 | ATaggatttccttccTT | CD80\|VTCN1 | |
| 624 | ATaggatttccttcCTT | CD80\|VTCN1 | |
| 625 | GAtaggatttccttccTT | CD80\|VTCN1 | |
| 626 | GAtaggatttccttcCTT | CD80\|VTCN1 | |
| 627 | GATaggatttccttcCTT | CD80\|VTCN1 | |
| 628 | TGataggatttccttcCTT | CD80\|VTCN1 | |
| 629 | TGAtaggatttccttccTT | CD80\|VTCN1 | |
| 630 | TGataggatttccttccTT | CD80\|VTCN1 | |
| 631 | ATgataggatttccttcCTT | CD80\|VTCN1 | |
| 632 | ATgataggatttccttccTT | CD80\|VTCN1 | |
| 633 | ATGataggatttccttccTT | CD80\|VTCN1 | |
| 634 | GCAtatgatagGATT | CD80\|VTCN1 | |
| 635 | GCATatgatagGATT | CD80\|VTCN1 | |
| 636 | GCatatgatagGATT | CD80\|VTCN1 | |
| 637 | AGCatatgatagGATT | CD80\|VTCN1 | |
| 638 | AGcatatgatagGATT | CD80\|VTCN1 | |
| 639 | AGCAtatgatagGATT | CD80\|VTCN1 | |
| 640 | TAGCatatgataggATT | CD80\|VTCN1 | |
| 641 | TAGCatatgataggATT | CD80\|VTCN1 | |
| 642 | TAGCatatgatagGATT | CD80\|VTCN1 | |
| 643 | TTTGcaaatctgaaaCTCT | CD80\|VTCN1 | |
| 644 | TTTGcaaatctgaaacTCT | CD80\|VTCN1 | |
| 645 | TTtgcaaatctgaaacTCT | CD80\|VTCN1 | |
| 646 | TTttgcaaatctgaaaCTCT | CD80\|VTCN1 | |
| 647 | TTttgcaaatctgaaactCT | CD80\|VTCN1 | |
| 648 | TTTTgcaaatctgaaaCTCT | CD80\|VTCN1 | |
| 649 | TTCAttttgcaaaTCT | CD80\|VTCN1 | |
| 650 | TTCAttttgcaaATCT | CD80\|VTCN1 | |
| 651 | TTCattttgcaaATCT | CD80\|VTCN1 | |
| 652 | TTTCattttgcaaATCT | CD80\|VTCN1 | |
| 653 | TTTCattttgcaaaTCT | CD80\|VTCN1 | |
| 654 | TTTcattttgcaaATCT | CD80\|VTCN1 | |
| 655 | TTTTcattttgcaaATCT | CD80\|VTCN1 | |
| 656 | TTTTcattttgcaaatCT | CD80\|VTCN1 | |
| 657 | TTTtcattttgcaaATCT | CD80\|VTCN1 | |
| 658 | TATgataggatttcCT | CD80\|VTCN1 | |
| 659 | TATgataggattTCCT | CD80\|VTCN1 | |
| 660 | TATGataggattTCCT | CD80\|VTCN1 | |
| 661 | ATATgataggattTCCT | CD80\|VTCN1 | |
| 662 | ATAtgataggatttCCT | CD80\|VTCN1 | |
| 663 | ATAtgataggatttcCT | CD80\|VTCN1 | |
| 664 | CAtatgataggatttcCT | CD80\|VTCN1 | |
| 665 | CATAtgataggatttcCT | CD80\|VTCN1 | |
| 666 | CAtatgataggatttCCT | CD80\|VTCN1 | |
| 667 | GCAtatgataggatttcCT | CD80\|VTCN1 | |
| 668 | GCatatgataggatttcCT | CD80\|VTCN1 | |
| 669 | GCatatgataggatttcCT | CD80\|VTCN1 | |
| 670 | AGCatatgataggatttcCT | CD80\|VTCN1 | |
| 671 | AGCatatgataggatttcCT | CD80\|VTCN1 | |
| 672 | AGCatatgataggatttcCT | CD80\|VTCN1 | |
| 673 | TAggatttccttcCT | CD80\|VTCN1 | |
| 674 | TAGgatttcctTCCT | CD80\|VTCN1 | |
| 675 | TAGgatttccttcCT | CD80\|VTCN1 | |
| 676 | ATaggatttccttcCT | CD80\|VTCN1 | |
| 677 | ATAggatttccttcCT | CD80\|VTCN1 | |
| 678 | ATAggatttcctTCCT | CD80\|VTCN1 | |
| 679 | GAtaggatttccttcCT | CD80\|VTCN1 | |
| 680 | GATAggatttccttcCT | CD80\|VTCN1 | |
| 681 | GATaggatttccttcCT | CD80\|VTCN1 | |
| 682 | TGAtaggatttccttcCT | CD80\|VTCN1 | |
| 683 | TGataggatttccttCCT | CD80\|VTCN1 | |
| 684 | TGataggatttccttcCT | CD80\|VTCN1 | |
| 685 | ATGataggatttccttcCT | CD80\|VTCN1 | |
| 686 | ATgataggatttccttCCT | CD80\|VTCN1 | |
| 687 | ATgataggatttccttcCT | CD80\|VTCN1 | |
| 688 | TAtgataggatttccttcCT | CD80\|VTCN1 | |
| 689 | TATgataggatttccttcCT | CD80\|VTCN1 | |
| 690 | TAtgataggatttccttCCT | CD80\|VTCN1 | |
| 691 | TTTgcaaatctgaAACT | CD80\|VTCN1 | |
| 692 | TTTGcaaatctgaaACT | CD80\|VTCN1 | |
| 693 | TTTGcaaatctgaAACT | CD80\|VTCN1 | |
| 694 | TTTTgcaaatctgaAACT | CD80\|VTCN1 | |
| 695 | TTTtgcaaatctgaAACT | CD80\|VTCN1 | |
| 696 | TTTTgcaaatctgaaACT | CD80\|VTCN1 | |
| 697 | ATTTtgcaaatctgaAACT | CD80\|VTCN1 | |
| 698 | ATTTtgcaaatctgaaACT | CD80\|VTCN1 | |
| 699 | ATTTtgcaaatctgaaaCT | CD80\|VTCN1 | |
| 700 | CATTttgcaaatctgaAACT | CD80\|VTCN1 | |
| 701 | CAttttgcaaatctgaaaCT | CD80\|VTCN1 | |
| 702 | CATtttgcaaatctgaAACT | CD80\|VTCN1 | |
| 703 | AAATctgaaactCTAT | CD80\|VTCN1 | |
| 704 | AAAtctgaaactCTAT | CD80\|VTCN1 | |
| 705 | AAATctgaaactcTAT | CD80\|VTCN1 | |
| 706 | CAaatctgaaactCTAT | CD80\|VTCN1 | |
| 707 | CAAatctgaaactCTAT | CD80\|VTCN1 | |
| 708 | CAAAtctgaaactCTAT | CD80\|VTCN1 | |
| 709 | GCAaatctgaaactctAT | CD80\|VTCN1 | |
| 710 | GCAAatctgaaactCTAT | CD80\|VTCN1 | |
| 711 | GCAAatctgaaactcTAT | CD80\|VTCN1 | |
| 712 | TGCAaatctgaaactCTAT | CD80\|VTCN1 | |
| 713 | TGCaaatctgaaactcTAT | CD80\|VTCN1 | |
| 714 | TGcaaatctgaaactcTAT | CD80\|VTCN1 | |
| 715 | TTGCaaatctgaaactCTAT | CD80\|VTCN1 | |
| 716 | TTGCaaatctgaaactctAT | CD80\|VTCN1 | |
| 717 | TTgcaaatctgaaactctAT | CD80\|VTCN1 | |
| 718 | GCATatgataGGAT | CD80\|VTCN1 | |
| 719 | GCAtatgataGGAT | CD80\|VTCN1 | |
| 720 | GCatatgataGGAT | CD80\|VTCN1 | |
| 721 | AGCAtatgataggAT | CD80\|VTCN1 | |
| 722 | AGCatatgataGGAT | CD80\|VTCN1 | |
| 723 | AGCAtatgataGGAT | CD80\|VTCN1 | |
| 724 | TAGCatatgataGGAT | CD80\|VTCN1 | |
| 725 | TAGCatatgatagGAT | CD80\|VTCN1 | |
| 726 | TAGcatatgatagGAT | CD80\|VTCN1 | |
| 727 | CATtttgcaaaTCTG | CD80\|VTCN1 | |
| 728 | CATTttgcaaatCTG | CD80\|VTCN1 | |
| 729 | CATTttgcaaaTCTG | CD80\|VTCN1 | |
| 730 | TCATtttgcaaaTCTG | CD80\|VTCN1 | |
| 731 | TCATtttgcaaatCTG | CD80\|VTCN1 | |
| 732 | TCAttttgcaaatCTG | CD80\|VTCN1 | |
| 733 | TTCAttttgcaaatCTG | CD80\|VTCN1 | |
| 734 | TTCAttttgcaaaTCTG | CD80\|VTCN1 | |
| 735 | TTCattttgcaaatCTG | CD80\|VTCN1 | |
| 736 | TTTCattttgcaaatcTG | CD80\|VTCN1 | |
| 737 | TTTCattttgcaaaTCTG | CD80\|VTCN1 | |
| 738 | TTTCattttgcaaatCTG | CD80\|VTCN1 | |
| 739 | TTTtcattttgcaaaTCTG | CD80\|VTCN1 | |
| 740 | TTTtcattttgcaaatcTG | CD80\|VTCN1 | |
| 741 | TTTTcattttgcaaaTCTG | CD80\|VTCN1 | |
| 742 | CTCctttgttcaCTG | CD80\|VTCN1 | |
| 743 | CTcctttgttcacTG | CD80\|VTCN1 | |
| 744 | CTCCtttgttcaCTG | CD80\|VTCN1 | |
| 745 | TAGcatatgaTAGG | CD80\|VTCN1 | |
| 746 | TAGCatatgaTAGG | CD80\|VTCN1 | |
| 747 | TAGCatatgatAGG | CD80\|VTCN1 | |
| 748 | TAggtgcttcTTAG | CD80\|VTCN1 | |
| 749 | TAGGtgcttcTTAG | CD80\|VTCN1 | |
| 750 | TAGGtgcttctTAG | CD80\|VTCN1 | |
| 751 | TTGTttgtttaaaaAG | CD80\|VTCN1 | |
| 752 | TTGTttgtttaaAAAG | CD80\|VTCN1 | |
| 753 | TTGTttgtttaaaAAG | CD80\|VTCN1 | |
| 754 | ATATgataggatTTC | CD80\|VTCN1 | |
| 755 | ATATgataggaTTTC | CD80\|VTCN1 | |
| 756 | ATAtgataggaTTTC | CD80\|VTCN1 | |
| 757 | CATAtgataggaTTTC | CD80\|VTCN1 | |
| 758 | CATAtgataggatTTC | CD80\|VTCN1 | |
| 759 | CATatgataggaTTTC | CD80\|VTCN1 | |
| 760 | GCatatgataggatTTC | CD80\|VTCN1 | |
| 761 | GCATatgataggatTTC | CD80\|VTCN1 | |
| 762 | GCATatgataggaTTTC | CD80\|VTCN1 | |
| 763 | AGCAtatgataggattTC | CD80\|VTCN1 | |
| 764 | AGCatatgataggatTTC | CD80\|VTCN1 | |
| 765 | AGCAtatgataggaTTTC | CD80\|VTCN1 | |
| 766 | TAGCatatgataggaTTTC | CD80\|VTCN1 | |
| 767 | TAgcatatgataggattTC | CD80\|VTCN1 | |
| 768 | TAGcatatgataggaTTTC | CD80\|VTCN1 | |
| 769 | ATGataggatttcCTTC | CD80\|VTCN1 | |
| 770 | ATgataggatttccTTC | CD80\|VTCN1 | |
| 771 | ATGAtaggatttcCTTC | CD80\|VTCN1 | |
| 772 | TATGataggatttcCTTC | CD80\|VTCN1 | |
| 773 | TATGataggatttcctTC | CD80\|VTCN1 | |
| 774 | TAtgataggatttcctTC | CD80\|VTCN1 | |
| 775 | ATATgataggatttcCTTC | CD80\|VTCN1 | |
| 776 | ATAtgataggatttccTTC | CD80\|VTCN1 | |
| 777 | ATatgataggatttcctTC | CD80\|VTCN1 | |
| 778 | CAtatgataggatttcctTC | CD80\|VTCN1 | |
| 779 | CATAtgataggatttccTTC | CD80\|VTCN1 | |
| 780 | CAtatgataggatttccTTC | CD80\|VTCN1 | |
| 781 | TTTGcaaatctgaaACTC | CD80\|VTCN1 | |
| 782 | TTTgcaaatctgaaaCTC | CD80\|VTCN1 | |
| 783 | TTTGcaaatctgaaaCTC | CD80\|VTCN1 | |
| 784 | TTTTgcaaatctgaaaCTC | CD80\|VTCN1 | |
| 785 | TTTTgcaaatctgaaacTC | CD80\|VTCN1 | |
| 786 | TTTTgcaaatctgaaACTC | CD80\|VTCN1 | |
| 787 | ATTttgcaaatctgaaACTC | CD80\|VTCN1 | |
| 788 | ATTTtgcaaatctgaaACTC | CD80\|VTCN1 | |
| 789 | ATTttgcaaatctgaaacTC | CD80\|VTCN1 | |
| 790 | TTTCattttgcaAATC | CD80\|VTCN1 | |
| 791 | TTTCattttgcaaATC | CD80\|VTCN1 | |
| 792 | TTTcattttgcaAATC | CD80\|VTCN1 | |
| 793 | TTTTcattttgcaAATC | CD80\|VTCN1 | |
| 794 | TTTtcattttgcaAATC | CD80\|VTCN1 | |
| 795 | TTTTcattttgcaaATC | CD80\|VTCN1 | |
| 796 | TCCtttgttcacTGC | CD80\|VTCN1 | |
| 797 | TCctttgttcacTGC | CD80\|VTCN1 | |
| 798 | TCctttgttcactGC | CD80\|VTCN1 | |
| 799 | CTcctttgttcactGC | CD80\|VTCN1 | |
| 800 | CTCctttgttcactGC | CD80\|VTCN1 | |
| 801 | CTCctttgttcacTGC | CD80\|VTCN1 | |
| 802 | TATGataggattTCC | CD80\|VTCN1 | |
| 803 | TATGataggatTTCC | CD80\|VTCN1 | |
| 804 | TATGataggatttCC | CD80\|VTCN1 | |
| 805 | ATatgataggatTTCC | CD80\|VTCN1 | |
| 806 | ATATgataggatTTCC | CD80\|VTCN1 | |
| 807 | ATATgataggattTCC | CD80\|VTCN1 | |
| 808 | CATAtgataggatTTCC | CD80\|VTCN1 | |
| 809 | CATatgataggatTTCC | CD80\|VTCN1 | |
| 810 | CAtatgataggatttCC | CD80\|VTCN1 | |
| 811 | GCatatgataggattTCC | CD80\|VTCN1 | |
| 812 | GCAtatgataggattTCC | CD80\|VTCN1 | |
| 813 | GCatatgataggatttCC | CD80\|VTCN1 | |
| 814 | AGcatatgataggattTCC | CD80\|VTCN1 | |
| 815 | AGcatatgataggatttCC | CD80\|VTCN1 | |
| 816 | AGCatatgataggatttCC | CD80\|VTCN1 | |
| 817 | TAgcatatgataggatTTCC | CD80\|VTCN1 | |
| 818 | TAgcatatgataggattTCC | CD80\|VTCN1 | |
| 819 | TAgcatatgataggatttCC | CD80\|VTCN1 | |
| 820 | ATaggatttccttCC | CD80\|VTCN1 | |
| 821 | ATAggatttcctTCC | CD80\|VTCN1 | |
| 822 | ATAGgatttccTTCC | CD80\|VTCN1 | |
| 823 | GATAggatttcctTCC | CD80\|VTCN1 | |
| 824 | GAtaggatttccttCC | CD80\|VTCN1 | |
| 825 | GAtaggatttcctTCC | CD80\|VTCN1 | |
| 826 | TGataggatttcctTCC | CD80\|VTCN1 | |
| 827 | TGataggatttccttCC | CD80\|VTCN1 | |
| 828 | TGATaggatttccttCC | CD80\|VTCN1 | |
| 829 | ATgataggatttccttCC | CD80\|VTCN1 | |
| 830 | ATGataggatttcctTCC | CD80\|VTCN1 | |
| 831 | ATgataggatttcctTCC | CD80\|VTCN1 | |
| 832 | TAtgataggatttccttCC | CD80\|VTCN1 | |
| 833 | TAtgataggatttcctTCC | CD80\|VTCN1 | |
| 834 | TATgataggatttccttCC | CD80\|VTCN1 | |
| 835 | ATAtgataggatttccttCC | CD80\|VTCN1 | |
| 836 | ATatgataggatttcctTCC | CD80\|VTCN1 | |
| 837 | ATatgataggatttccttCC | CD80\|VTCN1 | |
| 838 | TTTgcaaatctgAAAC | CD80\|VTCN1 | |
| 839 | TTTGcaaatctgaAAC | CD80\|VTCN1 | |
| 840 | TTTGcaaatctgAAAC | CD80\|VTCN1 | |
| 841 | TTTTgcaaatctgAAAC | CD80\|VTCN1 | |
| 842 | TTTTgcaaatctgaAAC | CD80\|VTCN1 | |
| 843 | TTTTgcaaatctgaaAC | CD80\|VTCN1 | |
| 844 | ATTTtgcaaatctgaAAC | CD80\|VTCN1 | |
| 845 | ATTTtgcaaatctgAAAC | CD80\|VTCN1 | |
| 846 | ATTttgcaaatctgAAAC | CD80\|VTCN1 | |
| 847 | CATTttgcaaatctgaaAC | CD80\|VTCN1 | |
| 848 | CATTttgcaaatctgAAAC | CD80\|VTCN1 | |
| 849 | CATTttgcaaatctgaAAC | CD80\|VTCN1 | |
| 850 | TCATtttgcaaatctgAAAC | CD80\|VTCN1 | |
| 851 | TCattttgcaaatctgAAAC | CD80\|VTCN1 | |
| 852 | TCATtttgcaaatctgaAAC | CD80\|VTCN1 | |
| 853 | CAAatctgaaacTCTA | CD80\|VTCN1 | |
| 854 | CAAAtctgaaacTCTA | CD80\|VTCN1 | |
| 855 | CAaatctgaaacTCTA | CD80\|VTCN1 | |
| 856 | GCAaatctgaaactcTA | CD80\|VTCN1 | |
| 857 | GCAAatctgaaacTCTA | CD80\|VTCN1 | |
| 858 | GCAaatctgaaacTCTA | CD80\|VTCN1 | |
| 859 | TGCAaatctgaaacTCTA | CD80\|VTCN1 | |
| 860 | TGCaaatctgaaactCTA | CD80\|VTCN1 | |
| 861 | TGcaaatctgaaactCTA | CD80\|VTCN1 | |
| 862 | TTGCaaatctgaaacTCTA | CD80\|VTCN1 | |
| 863 | TTGcaaatctgaaactcTA | CD80\|VTCN1 | |
| 864 | TTGCaaatctgaaactcTA | CD80\|VTCN1 | |
| 865 | TTtgcaaatctgaaactcTA | CD80\|VTCN1 | |
| 866 | TTTgcaaatctgaaactCTA | CD80\|VTCN1 | |
| 867 | TTTGcaaatctgaaacTCTA | CD80\|VTCN1 | |
| 868 | CATtttgcaaatcTGA | CD80\|VTCN1 | |
| 869 | CATtttgcaaatCTGA | CD80\|VTCN1 | |
| 870 | CATTttgcaaatCTGA | CD80\|VTCN1 | |
| 871 | TCAttttgcaaatCTGA | CD80\|VTCN1 | |
| 872 | TCATtttgcaaatCTGA | CD80\|VTCN1 | |
| 873 | TCattttgcaaatcTGA | CD80\|VTCN1 | |
| 874 | TTCattttgcaaatctGA | CD80\|VTCN1 | |
| 875 | TTCAttttgcaaatCTGA | CD80\|VTCN1 | |
| 876 | TTCAttttgcaaatcTGA | CD80\|VTCN1 | |
| 877 | TTTcattttgcaaatctGA | CD80\|VTCN1 | |
| 878 | TTTCattttgcaaatCTGA | CD80\|VTCN1 | |
| 879 | TTtcattttgcaaatCTGA | CD80\|VTCN1 | |
| 880 | TTTTcattttgcaaatCTGA | CD80\|VTCN1 | |
| 881 | TTttcattttgcaaatctGA | CD80\|VTCN1 | |
| 882 | TTTtcattttgcaaatcTGA | CD80\|VTCN1 | |
| 883 | AGCAtatgataGGA | CD80\|VTCN1 | |
| 884 | AGCAtatgatagGA | CD80\|VTCN1 | |
| 885 | AGCAtatgatAGGA | CD80\|VTCN1 | |
| 886 | TAGCatatgatAGGA | CD80\|VTCN1 | |
| 887 | TAGCatatgataGGA | CD80\|VTCN1 | |
| 888 | TAgcatatgatAGGA | CD80\|VTCN1 | |
| 889 | TTTTgcaaatcTGAA | CD80\|VTCN1 | |
| 890 | TTTtgcaaatcTGAA | CD80\|VTCN1 | |
| 891 | TTTTgcaaatctGAA | CD80\|VTCN1 | |
| 892 | ATTttgcaaatcTGAA | CD80\|VTCN1 | |
| 893 | ATTTtgcaaatcTGAA | CD80\|VTCN1 | |
| 894 | ATtttgcaaatcTGAA | CD80\|VTCN1 | |
| 895 | CATTttgcaaatctGAA | CD80\|VTCN1 | |
| 896 | CATtttgcaaatcTGAA | CD80\|VTCN1 | |
| 897 | CATTttgcaaatcTGAA | CD80\|VTCN1 | |
| 898 | TCATtttgcaaatcTGAA | CD80\|VTCN1 | |
| 899 | TCAttttgcaaatcTGAA | CD80\|VTCN1 | |
| 900 | TCATtttgcaaatctgAA | CD80\|VTCN1 | |
| 901 | TTCAttttgcaaatcTGAA | CD80\|VTCN1 | |
| 902 | TTCattttgcaaatctGAA | CD80\|VTCN1 | |
| 903 | TTCattttgcaaatcTGAA | CD80\|VTCN1 | |
| 904 | TTtcattttgcaaatctGAA | CD80\|VTCN1 | |
| 905 | TTTCattttgcaaatcTGAA | CD80\|VTCN1 | |
| 906 | TTTcattttgcaaatcTGAA | CD80\|VTCN1 | |
| 907 | TTTTgcaaatctgAAA | CD80\|VTCN1 | |
| 908 | TTTtgcaaatctGAAA | CD80\|VTCN1 | |
| 909 | TTTTgcaaatctGAAA | CD80\|VTCN1 | |
| 910 | ATTttgcaaatctGAAA | CD80\|VTCN1 | |
| 911 | ATTTtgcaaatctGAAA | CD80\|VTCN1 | |
| 912 | ATTTtgcaaatctgAAA | CD80\|VTCN1 | |
| 913 | CATTttgcaaatctGAAA | CD80\|VTCN1 | |
| 914 | CATTttgcaaatctgAAA | CD80\|VTCN1 | |
| 915 | CATtttgcaaatctGAAA | CD80\|VTCN1 | |
| 916 | TCAttttgcaaatctGAAA | CD80\|VTCN1 | |
| 917 | TCATtttgcaaatctgaAA | CD80\|VTCN1 | |
| 918 | TCATtttgcaaatctGAAA | CD80\|VTCN1 | |
| 919 | TTCAttttgcaaatctGAAA | CD80\|VTCN1 | |
| 920 | TTCAttttgcaaatctgAAA | CD80\|VTCN1 | |
| 921 | TTcattttgcaaatctGAAA | CD80\|VTCN1 | |
| 922 | CCTGtaattcacAC | CD86\|CD274 | |
| 923 | CCTGtaattcACAC | CD86\|CD274 | |
| 924 | CCTGtaattcaCAC | CD86\|CD274 | |
| 925 | CTAAttatggaaAAC | CD86\|CD274 | |
| 926 | CTAAttatggaAAAC | CD86\|CD274 | |
| 927 | CTAattatggaAAAC | CD86\|CD274 | |
| 928 | CCTAattatggaaaAC | CD86\|CD274 | |
| 929 | CCTAattatggaAAAC | CD86\|CD274 | |
| 930 | CCTAattatggaaAAC | CD86\|CD274 | |
| 931 | CCTGtaattcacACA | CD86\|CD274 | |
| 932 | CCTGtaattcaCACA | CD86\|CD274 | |
| 933 | CCtgtaattcacACA | CD86\|CD274 | |
| 934 | CTAattatggaaAACA | CD86\|CD274 | |
| 935 | CTAAttatggaaAACA | CD86\|CD274 | |
| 936 | CTAAttatggaaaACA | CD86\|CD274 | |
| 937 | CCTAattatggaaAACA | CD86\|CD274 | |
| 938 | CCTaattatggaaaACA | CD86\|CD274 | |
| 939 | CCTAattatggaaaACA | CD86\|CD274 | |
| 940 | TCTTtgtaaatgaAAA | CD86\|CD274 | |
| 941 | TCTTtgtaaatgAAAA | CD86\|CD274 | |
| 942 | TCTttgtaaatgAAAA | CD86\|CD274 | |
| 943 | CCTAattatggAAAA | CD86\|CD274 | |
| 944 | CCTaattatggAAAA | CD86\|CD274 | |
| 945 | CCTAattatggaAAA | CD86\|CD274 | |
| 946 | TTTggcttcccACT | CD86\|IDO1 | |
| 947 | TTTGgcttccCACT | CD86\|IDO1 | |
| 948 | TTtggcttcccaCT | CD86\|IDO1 | |
| 949 | ATttggcttcccaCT | CD86\|IDO1 | |
| 950 | ATTtggcttcccaCT | CD86\|IDO1 | |
| 951 | ATTtggcttccCACT | CD86\|IDO1 | |
| 952 | TTTGgcttcccaCTG | CD86\|IDO1 | |
| 953 | TTtggcttcccacTG | CD86\|IDO1 | |
| 954 | TTTggcttcccacTG | CD86\|IDO1 | |
| 955 | ATttggcttcccacTG | CD86\|IDO1 | |
| 956 | ATTtggcttcccACTG | CD86\|IDO1 | |
| 957 | ATTtggcttcccacTG | CD86\|IDO1 | |
| 958 | ATTtggcttcccAC | CD86\|IDO1 | |
| 959 | ATTTggcttccCAC | CD86\|IDO1 | |
| 960 | ATTTggcttcCCAC | CD86\|IDO1 | |
| 961 | TCtcttctccttctcTT | CD86\|LGALS9 | |
| 962 | TCTCttctccttctcTT | CD86\|LGALS9 | |
| 963 | TCTcttctccttctcTT | CD86\|LGALS9 | |
| 964 | TGgatgactgtCTG | CD86\|LGALS9 | |
| 965 | TGGAtgactgTCTG | CD86\|LGALS9 | |
| 966 | TGGAtgactgtCTG | CD86\|LGALS9 | |
| 967 | CTcttctccttctcTTC | CD86\|LGALS9 | |
| 968 | CTCttctccttctcTTC | CD86\|LGALS9 | |
| 969 | CTcttctccttctctTC | CD86\|LGALS9 | |
| 970 | TCtcttctccttctctTC | CD86\|LGALS9 | |
| 971 | TCTcttctccttctctTC | CD86\|LGALS9 | |
| 972 | TCtcttctccttctcTTC | CD86\|LGALS9 | |
| 973 | GGgtttggaactGG | CD86\|PDCD1 | |
| 974 | GGGTttggaaCTGG | CD86\|PDCD1 | |
| 975 | GGGtttggaacTGG | CD86\|PDCD1 | |
| 976 | TGGGttgatgAGAG | CD86\|PDCD1 | |
| 977 | TGGgttgatgAGAG | CD86\|PDCD1 | |
| 978 | TGGgttgatgagAG | CD86\|PDCD1 | |
| 979 | GGTtgatgagAGAG | CD86\|PDCD1 | |
| 980 | GGTTgatgagAGAG | CD86\|PDCD1 | |
| 981 | GGttgatgagAGAG | CD86\|PDCD1 | |
| 982 | GGGTtgatgagAGAG | CD86\|PDCD1 | |
| 983 | GGgttgatgagagAG | CD86\|PDCD1 | |
| 984 | GGgttgatgagAGAG | CD86\|PDCD1 | |
| 985 | TGggttgatgagagAG | CD86\|PDCD1 | |
| 986 | TGGGttgatgagagAG | CD86\|PDCD1 | |
| 987 | TGGgttgatgagAGAG | CD86\|PDCD1 | |
| 988 | GTctcaggcctgGGC | CD86\|PDCD1 | |
| 989 | GTctcaggcctggGC | CD86\|PDCD1 | |
| 990 | GTctcaggcctggGC | CD86\|PDCD1 | |
| 991 | TGcccagaatctCC | CD86\|PDCD1 | |
| 992 | TGCccagaatcTCC | CD86\|PDCD1 | |
| 993 | TGcccagaatcTCC | CD86\|PDCD1 | |
| 994 | GGGttgatgagaGA | CD86\|PDCD1 | |
| 995 | GGGTtgatgagAGA | CD86\|PDCD1 | |
| 996 | GGGTtgatgaGAGA | CD86\|PDCD1 | |
| 997 | TGGgttgatgagAGA | CD86\|PDCD1 | |
| 998 | TGGGttgatgaGAGA | CD86\|PDCD1 | |
| 999 | TGggttgatgagAGA | CD86\|PDCD1 | |
| 1000 | CCtctcccagtgTCT | CD86\|PDCD1LG2 | |
| 1001 | CCtctcccagtgtCT | CD86\|PDCD1LG2 | |
| 1002 | CCTctcccagtgtCT | CD86\|PDCD1LG2 | |
| 1003 | CTTtctgaagGTTG | CD86\|PDCD1LG2 | |
| 1004 | CTttctgaagGTTG | CD86\|PDCD1LG2 | |
| 1005 | CTTTctgaagGTTG | CD86\|PDCD1LG2 | |
| 1006 | GGGcccttccatGG | CD86\|PDCD1LG2 | |
| 1007 | GGgcccttccaTGG | CD86\|PDCD1LG2 | |
| 1008 | GGgcccttccatGG | CD86\|PDCD1LG2 | |
| 1009 | GAaactgaggctcaaagaAG | CD86\|PDCD1LG2 | |
| 1010 | GAAActgaggctcaaaGAAG | CD86\|PDCD1LG2 | |
| 1011 | GAAActgaggctcaaagAAG | CD86\|PDCD1LG2 | |
| 1012 | CTGGgcagataaaAG | CD86\|PDCD1LG2 | |
| 1013 | CTGGgcagataAAAG | CD86\|PDCD1LG2 | |
| 1014 | CTGGgcagataaAAG | CD86\|PDCD1LG2 | |
| 1015 | TTtttccatccatCC | CD86\|PDCD1LG2 | |
| 1016 | TTtttccatccATCC | CD86\|PDCD1LG2 | |
| 1017 | TTTTtccatccATCC | CD86\|PDCD1LG2 | |
| 1018 | CTctcccagtgtcTA | CD86\|PDCD1LG2 | |
| 1019 | CTctcccagtgTCTA | CD86\|PDCD1LG2 | |
| 1020 | CTCtcccagtgtcTA | CD86\|PDCD1LG2 | |
| 1021 | CCtctcccagtgtCTA | CD86\|PDCD1LG2 | |
| 1022 | CCtctcccagtgtcTA | CD86\|PDCD1LG2 | |
| 1023 | CCTctcccagtgtcTA | CD86\|PDCD1LG2 | |
| 1024 | TCAGgagctaaGAA | CD86\|PDCD1LG2 | |
| 1025 | TCAGgagctaAGAA | CD86\|PDCD1LG2 | |
| 1026 | TCAggagctaAGAA | CD86\|PDCD1LG2 | |
| 1027 | AAGAggtgacGGCT | CD86\|TNFRSF14 | |
| 1028 | AAGaggtgacGGCT | CD86\|TNFRSF14 | |
| 1029 | AAGaggtgacggCT | CD86\|TNFRSF14 | |
| 1030 | CAAGaggtgacggCT | CD86\|TNFRSF14 | |
| 1031 | CAagaggtgacggCT | CD86\|TNFRSF14 | |
| 1032 | CAAgaggtgacGGCT | CD86\|TNFRSF14 | |
| 1033 | AGAggtgacggCTG | CD86\|TNFRSF14 | |
| 1034 | AGAggtgacgGCTG | CD86\|TNFRSF14 | |
| 1035 | AGaggtgacggcTG | CD86\|TNFRSF14 | |
| 1036 | AAGAggtgacgGCTG | CD86\|TNFRSF14 | |
| 1037 | AAGaggtgacggcTG | CD86\|TNFRSF14 | |
| 1038 | AAGAggtgacggcTG | CD86\|TNFRSF14 | |
| 1039 | CAagaggtgacggCTG | CD86\|TNFRSF14 | |
| 1040 | CAagaggtgacggcTG | CD86\|TNFRSF14 | |
| 1041 | CAagaggtgacgGCTG | CD86\|TNFRSF14 | |
| 1042 | GAggtgacggctGG | CD86\|TNFRSF14 | |
| 1043 | GAGgtgacggcTGG | CD86\|TNFRSF14 | |
| 1044 | GAggtgacggcTGG | CD86\|TNFRSF14 | |
| 1045 | AGaggtgacggctGG | CD86\|TNFRSF14 | |
| 1046 | AGaggtgacggcTGG | CD86\|TNFRSF14 | |
| 1047 | AGAggtgacggcTGG | CD86\|TNFRSF14 | |
| 1048 | AAGaggtgacggCTGG | CD86\|TNFRSF14 | |
| 1049 | AAgaggtgacggctGG | CD86\|TNFRSF14 | |
| 1050 | AAgaggtgacggcTGG | CD86\|TNFRSF14 | |
| 1051 | CAAGaggtgacggctGG | CD86\|TNFRSF14 | |
| 1052 | CAAgaggtgacggctGG | CD86\|TNFRSF14 | |
| 1053 | CAagaggtgacggctGG | CD86\|TNFRSF14 | |
| 1054 | CAagaggtgaCGGC | CD86\|TNFRSF14 | |
| 1055 | CAagaggtgacgGC | CD86\|TNFRSF14 | |
| 1056 | CAAGaggtgaCGGC | CD86\|TNFRSF14 | |
| 1057 | CCAGggcttcatCA | CD86\|TNFRSF14 | |
| 1058 | CCagggcttcatCA | CD86\|TNFRSF14 | |
| 1059 | CCagggcttcaTCA | CD86\|TNFRSF14 | |
| 1060 | TACAcagtagtGTG | CD86\|VTCN1 | |
| 1061 | TACacagtagTGTG | CD86\|VTCN1 | |
| 1062 | TACAcagtagTGTG | CD86\|VTCN1 | |
| 1063 | GAgccatgcccATC | CD86\|VTCN1 | |
| 1064 | GAgccatgccCATC | CD86\|VTCN1 | |
| 1065 | GAgccatgcccaTC | CD86\|VTCN1 | |
| 1066 | GGagccatgcccATC | CD86\|VTCN1 | |
| 1067 | GGagccatgcccaTC | CD86\|VTCN1 | |
| 1068 | GGAgccatgcccaTC | CD86\|VTCN1 | |
| 1069 | CATCtttggaCTGC | CD86\|VTCN1 | |
| 1070 | CATctttggactGC | CD86\|VTCN1 | |
| 1071 | CATCtttggacTGC | CD86\|VTCN1 | |
| 1072 | ATCTctatcctgGC | CD86\|VTCN1 | |
| 1073 | ATCTctatccTGGC | CD86\|VTCN1 | |
| 1074 | ATctctatcctgGC | CD86\|VTCN1 | |
| 1075 | GAgccatgcccaTCC | CD86\|VTCN1 | |
| 1076 | GAgccatgcccatCC | CD86\|VTCN1 | |
| 1077 | GAGccatgcccatCC | CD86\|VTCN1 | |
| 1078 | GGagccatgcccATCC | CD86\|VTCN1 | |
| 1079 | GGagccatgcccaTCC | CD86\|VTCN1 | |
| 1080 | GGagccatgcccatCC | CD86\|VTCN1 | |
| 1081 | AGAgttaggagaAGA | CD86\|VTCN1 | |
| 1082 | AGAGttaggagAAGA | CD86\|VTCN1 | |
| 1083 | AGAGttaggagaAGA | CD86\|VTCN1 | |
| 1084 | GATctggctgCTAA | CD86\|VTCN1 | |
| 1085 | GATCtggctgCTAA | CD86\|VTCN1 | |
| 1086 | GATCtggctgctAA | CD86\|VTCN1 | |
| 1087 | GCAtttgtaaaCCAA | CD86\|VTCN1 | |
| 1088 | GCATttgtaaaCCAA | CD86\|VTCN1 | |
| 1089 | GCatttgtaaaCCAA | CD86\|VTCN1 | |
| 1090 | GAGActtggcaaAA | CD86\|VTCN1 | |
| 1091 | GAGActtggcaAAA | CD86\|VTCN1 | |
| 1092 | GAGActtggcAAAA | CD86\|VTCN1 | |
| 1093 | GAGGaatacggAAG | CTLA4\|CD274 | |
| 1094 | GAGGaatacgGAAG | CTLA4\|CD274 | |
| 1095 | GAGgaatacgGAAG | CTLA4\|CD274 | |
| 1096 | TGAggaatacgGAAG | CTLA4\|CD274 | |
| 1097 | TGAGgaatacgGAAG | CTLA4\|CD274 | |
| 1098 | TGAGgaatacggaAG | CTLA4\|CD274 | |
| 1099 | CCAtgatccaaTTC | CTLA4\|CD274 | |
| 1100 | CCATgatccaATTC | CTLA4\|CD274 | |
| 1101 | CCATgatccaaTTC | CTLA4\|CD274 | |
| 1102 | TGAGgaatacgGAA | CTLA4\|CD274 | |
| 1103 | TGAGgaatacGGAA | CTLA4\|CD274 | |
| 1104 | TGAggaatacGGAA | CTLA4\|CD274 | |
| 1105 | CCAAcacctgtGAT | CTLA4\|CD86 | |
| 1106 | CCaacacctgtgAT | CTLA4\|CD86 | |
| 1107 | CCAAcacctgTGAT | CTLA4\|CD86 | |
| 1108 | ACcaacacctgtgAT | CTLA4\|CD86 | |
| 1109 | ACCAacacctgtgAT | CTLA4\|CD86 | |
| 1110 | ACCAacacctgTGAT | CTLA4\|CD86 | |
| 1111 | TACcaacacctgtGAT | CTLA4\|CD86 | |
| 1112 | TAccaacacctgtgAT | CTLA4\|CD86 | |
| 1113 | TACCaacacctgtGAT | CTLA4\|CD86 | |
| 1114 | TACCaacaccTGTG | CTLA4\|CD86 | |
| 1115 | TACCaacacctGTG | CTLA4\|CD86 | |
| 1116 | TAccaacacctGTG | CTLA4\|CD86 | |
| 1117 | TAccaacacctGTGA | CTLA4\|CD86 | |
| 1118 | TAccaacacctgtGA | CTLA4\|CD86 | |
| 1119 | TACCaacacctGTGA | CTLA4\|CD86 | |
| 1120 | ACTctagggctcTA | CTLA4\|LGALS9 | |
| 1121 | ACTCtagggcTCTA | CTLA4\|LGALS9 | |
| 1122 | ACTctagggcTCTA | CTLA4\|LGALS9 | |
| 1123 | GACacaacatcTTTT | HMOX1\|CD80 | |
| 1124 | GACAcaacatctTTT | HMOX1\|CD80 | |
| 1125 | GACAcaacatcTTTT | HMOX1\|CD80 | |
| 1126 | GACAcaacatCTTT | HMOX1\|CD80 | |
| 1127 | GACAcaacatcTTT | HMOX1\|CD80 | |
| 1128 | GACacaacatCTTT | HMOX1\|CD80 | |
| 1129 | TTAgaagcagGACA | HMOX1\|CD80 | |
| 1130 | TTAGaagcagGACA | HMOX1\|CD80 | |
| 1131 | TTAGaagcaggACA | HMOX1\|CD80 | |
| 1132 | TTTagaagcagGACA | HMOX1\|CD80 | |
| 1133 | TTTAgaagcagGACA | HMOX1\|CD80 | |
| 1134 | TTTAgaagcaggACA | HMOX1\|CD80 | |
| 1135 | CAgccttgtggcTGG | HMOX1\|CD86 | |
| 1136 | CAGccttgtggctGG | HMOX1\|CD86 | |
| 1137 | CAgccttgtggctGG | HMOX1\|CD86 | |
| 1138 | CCCtgcctgctctTG | HMOX1\|PDCD1LG2 | |
| 1139 | CCctgcctgctcTTG | HMOX1\|PDCD1LG2 | |
| 1140 | CCctgcctgctctTG | HMOX1\|PDCD1LG2 | |
| 1141 | CCtgcctgctctTGC | HMOX1\|PDCD1LG2 | |
| 1142 | CCTgcctgctcttGC | HMOX1\|PDCD1LG2 | |
| 1143 | CCtgcctgctcttGC | HMOX1\|PDCD1LG2 | |
| 1144 | CCctgcctgctcttGC | HMOX1\|PDCD1LG2 | |
| 1145 | CCCtgcctgctcttGC | HMOX1\|PDCD1LG2 | |
| 1146 | CCctgcctgctctTGC | HMOX1\|PDCD1LG2 | |
| 1147 | AGttccctcaccTGC | HMOX1\|PDCD1LG2 | |
| 1148 | AGttccctcacctGC | HMOX1\|PDCD1LG2 | |
| 1149 | AGTtccctcacctGC | HMOX1\|PDCD1LG2 | |
| 1150 | CCCAcgctgatgTT | HMOX1\|VTCN1 | |
| 1151 | CCcacgctgatGTT | HMOX1\|VTCN1 | |
| 1152 | CCcacgctgatgTT | HMOX1\|VTCN1 | |
| 1153 | TAAGccaatgaACAA | PDCD1LG2\|IDO1 | |
| 1154 | TAAGccaatgaaCAA | PDCD1LG2\|IDO1 | CRM0198 |
| 1155 | TAAgccaatgaACAA | PDCD1LG2\|IDO1 | |
| 1156 | CCCacccaggaagGA | PDCD1\|LGALS9 | |
| 1157 | CCcacccaggaaGGA | PDCD1\|LGALS9 | |
| 1158 | CCcacccaggaagGA | PDCD1\|LGALS9 | |
| 1159 | GCAgaacactgGTG | PDCD1\|PDCD1LG2 | |
| 1160 | GCagaacactgGTG | PDCD1\|PDCD1LG2 | |
| 1161 | GCAGaacactGGTG | PDCD1\|PDCD1LG2 | |
| 1162 | CTcaacccttTTCC | PDCD1\|PDCD1LG2 | |
| 1163 | CTcaacccttttCC | PDCD1\|PDCD1LG2 | |
| 1164 | CTCAacccttTTCC | PDCD1\|PDCD1LG2 | |
| 1165 | CCCacaaagggcCT | PDCD1\|VTCN1 | |
| 1166 | CCcacaaagggcCT | PDCD1\|VTCN1 | |
| 1167 | CCcacaaagggCCT | PDCD1\|VTCN1 | |
| 1168 | CCacaaagggccTG | PDCD1\|VTCN1 | |
| 1169 | CCACaaagggcCTG | PDCD1\|VTCN1 | |
| 1170 | CCacaaagggcCTG | PDCD1\|VTCN1 | |
| 1171 | CCCacaaagggccTG | PDCD1\|VTCN1 | |
| 1172 | CCcacaaagggccTG | PDCD1\|VTCN1 | |
| 1173 | CCcacaaagggcCTG | PDCD1\|VTCN1 | |
| 1174 | AAAGatgcttcaGAG | PDCD1\|VTCN1 | |
| 1175 | AAAGatgcttcAGAG | PDCD1\|VTCN1 | |
| 1176 | AAAgatgcttcAGAG | PDCD1\|VTCN1 | |
| 1177 | CAAGcgcttcaCAG | TNFRSF14\|IDO1 | |
| 1178 | CAagcgcttcaCAG | TNFRSF14\|IDO1 | |
| 1179 | CAAGcgcttcACAG | TNFRSF14\|IDO1 | |
| 1180 | GGgcaggctccctGT | TNFRSF14\|VTCN1 | |
| 1181 | GGgcaggctcccTGT | TNFRSF14\|VTCN1 | |
| 1182 | GGGcaggctccctGT | TNFRSF14\|VTCN1 | |
| 1183 | CAggccctgccccCAG | TNFRSF14\|VTCN1 | |
| 1184 | CAggccctgcccccAG | TNFRSF14\|VTCN1 | |
| 1185 | CAGgccctgcccccAG | TNFRSF14\|VTCN1 | |
| 1186 | CCacagagctggcCC | TNFRSF14\|VTCN1 | |
| 1187 | CCAcagagctggcCC | TNFRSF14\|VTCN1 | |
| 1188 | CCacagagctggCCC | TNFRSF14\|VTCN1 | |
| 1189 | CCcacagagctggCCC | TNFRSF14\|VTCN1 | |
| 1190 | CCcacagagctggcCC | TNFRSF14\|VTCN1 | |
| 1191 | CCCacagagctggcCC | TNFRSF14\|VTCN1 | |
| 1192 | ACTCaggactTGAT | VTCN1\|CD274 | |
| 1193 | ACTcaggactTGAT | VTCN1\|CD274 | |
| 1194 | ACTCaggacttGAT | VTCN1\|CD274 | |
| 1195 | CACTcaggactTGAT | VTCN1\|CD274 | |
| 1196 | CACtcaggactTGAT | VTCN1\|CD274 | |
| 1197 | CACtcaggacttgAT | VTCN1\|CD274 | |
| 1198 | ACTCaggacttgaTG | VTCN1\|CD274 | |
| 1199 | ACTcaggacttGATG | VTCN1\|CD274 | |
| 1200 | ACTCaggacttGATG | VTCN1\|CD274 | |
| 1201 | CActcaggacttgaTG | VTCN1\|CD274 | |
| 1202 | CACTcaggacttgATG | VTCN1\|CD274 | |
| 1203 | CACTcaggacttGATG | VTCN1\|CD274 | |
| 1204 | CTCAggacttgATGG | VTCN1\|CD274 | |
| 1205 | CTCaggacttgATGG | VTCN1\|CD274 | |
| 1206 | CTCaggacttgatGG | VTCN1\|CD274 | |
| 1207 | ACTcaggacttgaTGG | VTCN1\|CD274 | |
| 1208 | ACtcaggacttgatGG | VTCN1\|CD274 | |
| 1209 | ACTCaggacttgATGG | VTCN1\|CD274 | |
| 1210 | CActcaggacttgatGG | VTCN1\|CD274 | |
| 1211 | CACTcaggacttgaTGG | VTCN1\|CD274 | |
| 1212 | CACtcaggacttgatGG | VTCN1\|CD274 | |
| 1213 | CACTcaggactTGA | VTCN1\|CD274 | |
| 1214 | CACTcaggacttGA | VTCN1\|CD274 | |
| 1215 | CACTcaggacTTGA | VTCN1\|CD274 | |
| 1216 | GCATtttcagaagAG | VTCN1\|IDO1 | |
| 1217 | GCAttttcagaAGAG | VTCN1\|IDO1 | |
| 1218 | GCATtttcagaAGAG | VTCN1\|IDO1 | |
| 1219 | TGCattttcagaAGAG | VTCN1\|IDO1 | |
| 1220 | TGCattttcagaagAG | VTCN1\|IDO1 | |
| 1221 | TGCAttttcagaAGAG | VTCN1\|IDO1 | |
| 1222 | TTgcattttcagaaGAG | VTCN1\|IDO1 | |
| 1223 | TTGcattttcagaAGAG | VTCN1\|IDO1 | |
| 1224 | TTGCattttcagaAGAG | VTCN1\|IDO1 | |
| 1225 | TTTGcattttcagaAGAG | VTCN1\|IDO1 | |
| 1226 | TTTGcattttcagaaGAG | VTCN1\|IDO1 | |
| 1227 | TTTgcattttcagaagAG | VTCN1\|IDO1 | |
| 1228 | TTGCattttcaGAAG | VTCN1\|IDO1 | |
| 1229 | TTGcattttcaGAAG | VTCN1\|IDO1 | |
| 1230 | TTGCattttcagAAG | VTCN1\|IDO1 | |
| 1231 | TTTgcattttcaGAAG | VTCN1\|IDO1 | |
| 1232 | TTTGcattttcagAAG | VTCN1\|IDO1 | |
| 1233 | TTTGcattttcaGAAG | VTCN1\|IDO1 | |
| 1234 | ACCTgtctggAAAC | VTCN1\|IDO1 | |
| 1235 | ACCtgtctggAAAC | VTCN1\|IDO1 | |
| 1236 | ACCTgtctggaAAC | VTCN1\|IDO1 | |
| 1237 | TTGCattttcagaAGA | VTCN1\|IDO1 | |
| 1238 | TTGCattttcagaAGA | VTCN1\|IDO1 | |
| 1239 | TTGCattttcagAAGA | VTCN1\|IDO1 | |
| 1240 | TTtgcattttcagAAGA | VTCN1\|IDO1 | |
| 1241 | TTTGcattttcagAAGA | VTCN1\|IDO1 | |
| 1242 | TTTGcattttcagaAGA | VTCN1\|IDO1 | |
| 1243 | GGATggctatgaGAA | VTCN1\|IDO1 | |
| 1244 | GGatggctatgaGAA | VTCN1\|IDO1 | |
| 1245 | GGATggctatgAGAA | VTCN1\|IDO1 | |
| 1246 | CTgccacccaggAGT | VTCN1\|LGALS9 | |
| 1247 | CTGccacccaggaGT | VTCN1\|LGALS9 | |
| 1248 | CTgccacccaggaGT | VTCN1\|LGALS9 | |
| 1249 | TGcctcattggcCT | VTCN1\|LGALS9 | |
| 1250 | TGCctcattggcCT | VTCN1\|LGALS9 | |
| 1251 | TGcctcattggCCT | VTCN1\|LGALS9 | |
| 1252 | CTgcctcattggCCT | VTCN1\|LGALS9 | |
| 1253 | CTgcctcattggcCT | VTCN1\|LGALS9 | |
| 1254 | CTGcctcattggcCT | VTCN1\|LGALS9 | |
| 1255 | ACtgcctcattggCCT | VTCN1\|LGALS9 | |
| 1256 | ACTgcctcattggcCT | VTCN1\|LGALS9 | |
| 1257 | ACtgcctcattggcCT | VTCN1\|LGALS9 | |
| 1258 | CTTTtagatgtTGG | VTCN1\|LGALS9 | |
| 1259 | CTTTtagatgTTGG | VTCN1\|LGALS9 | |
| 1260 | CTTttagatgTTGG | VTCN1\|LGALS9 | |
| 1261 | ACTGcctcattGGC | VTCN1\|LGALS9 | |
| 1262 | ACtgcctcattGGC | VTCN1\|LGALS9 | |
| 1263 | ACtgcctcattgGC | VTCN1\|LGALS9 | |
| 1264 | ACTgcctcattggCC | VTCN1\|LGALS9 | |
| 1265 | ACtgcctcattgGCC | VTCN1\|LGALS9 | |
| 1266 | ACtgcctcattggCC | VTCN1\|LGALS9 | |
| 1267 | TGgcttctttttgtTT | VTCN1\|PDCD1LG2 | |
| 1268 | TGGcttctttttgTTT | VTCN1\|PDCD1LG2 | |
| 1269 | TGGCttctttttGTTT | VTCN1\|PDCD1LG2 | |
| 1270 | CTgtctggaaaCCTT | VTCN1\|PDCD1LG2 | |
| 1271 | CTGtctggaaaccTT | VTCN1\|PDCD1LG2 | |
| 1272 | CTGTctggaaaCCTT | VTCN1\|PDCD1LG2 | |
| 1273 | CCTgtctggaaacCTT | VTCN1\|PDCD1LG2 | |
| 1274 | CCtgtctggaaacCTT | VTCN1\|PDCD1LG2 | |
| 1275 | CCtgtctggaaaccTT | VTCN1\|PDCD1LG2 | |
| 1276 | TCTGaggcagaATGT | VTCN1\|PDCD1LG2 | |
| 1277 | TCTGaggcagaaTGT | VTCN1\|PDCD1LG2 | |
| 1278 | TCTgaggcagaatGT | VTCN1\|PDCD1LG2 | |
| 1279 | TATggtgtatACGT | VTCN1\|PDCD1LG2 | |
| 1280 | TATGgtgtatACGT | VTCN1\|PDCD1LG2 | |
| 1281 | TATGgtgtataCGT | VTCN1\|PDCD1LG2 | |
| 1282 | CTATggtgtataCGT | VTCN1\|PDCD1LG2 | |
| 1283 | CTATggtgtatACGT | VTCN1\|PDCD1LG2 | |
| 1284 | CTatggtgtataCGT | VTCN1\|PDCD1LG2 | |
| 1285 | TCTAtggtgtatACGT | VTCN1\|PDCD1LG2 | |
| 1286 | TCTatggtgtatACGT | VTCN1\|PDCD1LG2 | |
| 1287 | TCTatggtgtatacGT | VTCN1\|PDCD1LG2 | |
| 1288 | TTCtatggtgtatACGT | VTCN1\|PDCD1LG2 | |
| 1289 | TTCtatggtgtatacGT | VTCN1\|PDCD1LG2 | |
| 1290 | TTCTatggtgtatACGT | VTCN1\|PDCD1LG2 | |
| 1291 | ATTctatggtgtataCGT | VTCN1\|PDCD1LG2 | |
| 1292 | ATTCtatggtgtatACGT | VTCN1\|PDCD1LG2 | |
| 1293 | ATtctatggtgtatacGT | VTCN1\|PDCD1LG2 | |
| 1294 | TAttctatggtgtatacGT | VTCN1\|PDCD1LG2 | |
| 1295 | TATTctatggtgtatACGT | VTCN1\|PDCD1LG2 | |
| 1296 | TAttctatggtgtataCGT | VTCN1\|PDCD1LG2 | |
| 1297 | GTAttctatggtgtatacGT | VTCN1\|PDCD1LG2 | |
| 1298 | GTattctatggtgtatacGT | VTCN1\|PDCD1LG2 | |
| 1299 | GTattctatggtgtatacGT | VTCN1\|PDCD1LG2 | |
| 1300 | GAgtccagatCAGT | VTCN1\|PDCD1LG2 | |
| 1301 | GAgtccagatcaGT | VTCN1\|PDCD1LG2 | |
| 1302 | GAGTccagatCAGT | VTCN1\|PDCD1LG2 | |
| 1303 | TGagtccagatcaGT | VTCN1\|PDCD1LG2 | |
| 1304 | TGagtccagatCAGT | VTCN1\|PDCD1LG2 | |
| 1305 | TGAgtccagatCAGT | VTCN1\|PDCD1LG2 | |
| 1306 | AAGGaagttattTCT | VTCN1\|PDCD1LG2 | |
| 1307 | AAGGaagttatTTCT | VTCN1\|PDCD1LG2 | |
| 1308 | AAGgaagttatTTCT | VTCN1\|PDCD1LG2 | |
| 1309 | GATttttgaaaTCCT | VTCN1\|PDCD1LG2 | |
| 1310 | GATTtttgaaaTCCT | VTCN1\|PDCD1LG2 | |
| 1311 | GATTtttgaaatCCT | VTCN1\|PDCD1LG2 | |
| 1312 | CAAcagtggaCCCT | VTCN1\|PDCD1LG2 | |
| 1313 | CAacagtggaccCT | VTCN1\|PDCD1LG2 | |
| 1314 | CAAcagtggacCCT | VTCN1\|PDCD1LG2 | |
| 1315 | CCTgtctggaaaCCT | VTCN1\|PDCD1LG2 | |
| 1316 | CCTgtctggaaacCT | VTCN1\|PDCD1LG2 | |
| 1317 | CCtgtctggaaacCT | VTCN1\|PDCD1LG2 | |
| 1318 | TGCCtctgaggaCT | VTCN1\|PDCD1LG2 | |
| 1319 | TGcctctgaggaCT | VTCN1\|PDCD1LG2 | |
| 1320 | TGCctctgaggaCT | VTCN1\|PDCD1LG2 | |
| 1321 | GGGTagttttGGAT | VTCN1\|PDCD1LG2 | |
| 1322 | GGGtagttttggAT | VTCN1\|PDCD1LG2 | |
| 1323 | GGGtagttttGGAT | VTCN1\|PDCD1LG2 | |
| 1324 | TAtggtgtataCGTG | VTCN1\|PDCD1LG2 | |
| 1325 | TATggtgtataCGTG | VTCN1\|PDCD1LG2 | |
| 1326 | TATGgtgtataCGTG | VTCN1\|PDCD1LG2 | |
| 1327 | CTATggtgtataCGTG | VTCN1\|PDCD1LG2 | |
| 1328 | CTATggtgtatacGTG | VTCN1\|PDCD1LG2 | |
| 1329 | CTAtggtgtatacgTG | VTCN1\|PDCD1LG2 | |
| 1330 | TCTAtggtgtatacgTG | VTCN1\|PDCD1LG2 | |
| 1331 | TCtatggtgtatacgTG | VTCN1\|PDCD1LG2 | |
| 1332 | TCTAtggtgtataCGTG | VTCN1\|PDCD1LG2 | |
| 1333 | TTctatggtgtatacgTG | VTCN1\|PDCD1LG2 | |
| 1334 | TTCTatggtgtataCGTG | VTCN1\|PDCD1LG2 | |
| 1335 | TTCtatggtgtatacGTG | VTCN1\|PDCD1LG2 | |
| 1336 | ATtctatggtgtatacgTG | VTCN1\|PDCD1LG2 | |
| 1337 | ATTctatggtgtataCGTG | VTCN1\|PDCD1LG2 | |
| 1338 | ATtctatggtgtatacGTG | VTCN1\|PDCD1LG2 | |
| 1339 | TAttctatggtgtatacGTG | VTCN1\|PDCD1LG2 | |
| 1340 | TAttctatggtgtatacgTG | VTCN1\|PDCD1LG2 | |
| 1341 | TATTctatggtgtatacGTG | VTCN1\|PDCD1LG2 | |
| 1342 | TCTgaggCagAATG | VTCN1\|PDCD1LG2 | |
| 1343 | TCTGaggcagAATG | VTCN1\|PDCD1LG2 | |
| 1344 | TCTGaggcagaATG | VTCN1\|PDCD1LG2 | |
| 1345 | CTAtggtgtaTACG | VTCN1\|PDCD1LG2 | |
| 1346 | CTATggtgtatACG | VTCN1\|PDCD1LG2 | |
| 1347 | CTATggtgtaTACG | VTCN1\|PDCD1LG2 | |
| 1348 | TCTAtggtgtaTACG | VTCN1\|PDCD1LG2 | |
| 1349 | TCTatggtgtaTACG | VTCN1\|PDCD1LG2 | |
| 1350 | TCTAtggtgtataCG | VTCN1\|PDCD1LG2 | |
| 1351 | TTCtatggtgtaTACG | VTCN1\|PDCD1LG2 | |
| 1352 | TTCTatggtgtaTACG | VTCN1\|PDCD1LG2 | |
| 1353 | TTCtatggtgtaTACG | VTCN1\|PDCD1LG2 | |
| 1354 | ATTCtatggtgtaTACG | VTCN1\|PDCD1LG2 | |
| 1355 | ATTCtatggtgtaTACG | VTCN1\|PDCD1LG2 | |
| 1356 | ATTctatggtgtatACG | VTCN1\|PDCD1LG2 | |
| 1357 | TAttctatggtgtatACG | VTCN1\|PDCD1LG2 | |
| 1358 | TATTctatggtgtaTACG | VTCN1\|PDCD1LG2 | |
| 1359 | TATtctatggtgtaTACG | VTCN1\|PDCD1LG2 | |
| 1360 | GTAttctatggtgtataCG | VTCN1\|PDCD1LG2 | |
| 1361 | GTAttctatggtgtaTACG | VTCN1\|PDCD1LG2 | |
| 1362 | GTattctatggtgtataCG | VTCN1\|PDCD1LG2 | |
| 1363 | AGtattctatggtgtataCG | VTCN1\|PDCD1LG2 | |
| 1364 | AGtattctatggtgtatACG | VTCN1\|PDCD1LG2 | |
| 1365 | AGTAttctatggtgtataCG | VTCN1\|PDCD1LG2 | |
| 1366 | GCATtgcactTTAG | VTCN1\|PDCD1LG2 | |
| 1367 | GCATtgcacttTAG | VTCN1\|PDCD1LG2 | |
| 1368 | GCattgcacttTAG | VTCN1\|PDCD1LG2 | |
| 1369 | TGAgtccagatcAG | VTCN1\|PDCD1LG2 | |
| 1370 | TGAGtccagatCAG | VTCN1\|PDCD1LG2 | |
| 1371 | TGAGtccagaTCAG | VTCN1\|PDCD1LG2 | |
| 1372 | TGAgagatgtTATC | VTCN1\|PDCD1LG2 | |
| 1373 | TGAGagatgtTATC | VTCN1\|PDCD1LG2 | |
| 1374 | TGAGagatgttATC | VTCN1\|PDCD1LG2 | |
| 1375 | CTGagagatgtTATC | VTCN1\|PDCD1LG2 | |
| 1376 | CTGAgagatgttATC | VTCN1\|PDCD1LG2 | |
| 1377 | CTGAgagatgtTATC | VTCN1\|PDCD1LG2 | |
| 1378 | TATGgtgtatacgTGC | VTCN1\|PDCD1LG2 | |
| 1379 | TAtggtgtatacGTGC | VTCN1\|PDCD1LG2 | |
| 1380 | TAtggtgtatacgtGC | VTCN1\|PDCD1LG2 | |
| 1381 | CTatggtgtatacgtGC | VTCN1\|PDCD1LG2 | |
| 1382 | CTatggtgtatacGTGC | VTCN1\|PDCD1LG2 | |
| 1383 | CTAtggtgtatacgtGC | VTCN1\|PDCD1LG2 | |
| 1384 | TCTAtggtgtatacgtGC | VTCN1\|PDCD1LG2 | |
| 1385 | TCTatggtgtatacgtGC | VTCN1\|PDCD1LG2 | |
| 1386 | TCtatggtgtatacgtGC | VTCN1\|PDCD1LG2 | |
| 1387 | TTctatggtgtatacgtGC | VTCN1\|PDCD1LG2 | |
| 1388 | TTctatggtgtatacgTGC | VTCN1\|PDCD1LG2 | |
| 1389 | TTCtatggtgtatacgtGC | VTCN1\|PDCD1LG2 | |
| 1390 | ATtctatggtgtatacgTGC | VTCN1\|PDCD1LG2 | |
| 1391 | ATtctatggtgtatacgtGC | VTCN1\|PDCD1LG2 | |
| 1392 | ATTCtatggtgtatacgtGC | VTCN1\|PDCD1LG2 | |
| 1393 | TATggtgtatacgtgCC | VTCN1\|PDCD1LG2 | |
| 1394 | TAtggtgtatacgtGCC | VTCN1\|PDCD1LG2 | |
| 1395 | TAtggtgtatacgtgCC | VTCN1\|PDCD1LG2 | |
| 1396 | CTatggtgtatacgtgCC | VTCN1\|PDCD1LG2 | |
| 1397 | CTatggtgtatacgtGCC | VTCN1\|PDCD1LG2 | |
| 1398 | CTAtggtgtatacgtgCC | VTCN1\|PDCD1LG2 | |
| 1399 | TCtatggtgtatacgtGCC | VTCN1\|PDCD1LG2 | |
| 1400 | TCTatggtgtatacgtgCC | VTCN1\|PDCD1LG2 | |
| 1401 | TCtatggtgtatacgtgCC | VTCN1\|PDCD1LG2 | |
| 1402 | TTctatggtgtatacgtgCC | VTCN1\|PDCD1LG2 | |
| 1403 | TTctatggtgtatacgtGCC | VTCN1\|PDCD1LG2 | |
| 1404 | TTCtatggtgtatacgtgCC | VTCN1\|PDCD1LG2 | |
| 1405 | TATTctatggtgtATAC | VTCN1\|PDCD1LG2 | |
| 1406 | TATtctatggtgtaTAC | VTCN1\|PDCD1LG2 | |
| 1407 | TATtctatggtgtATAC | VTCN1\|PDCD1LG2 | |
| 1408 | GTATtctatggtgtATAC | VTCN1\|PDCD1LG2 | |
| 1409 | GTAttctatggtgtATAC | VTCN1\|PDCD1LG2 | |
| 1410 | GTattctatggtgtatAC | VTCN1\|PDCD1LG2 | |
| 1411 | AGtattctatggtgtatAC | VTCN1\|PDCD1LG2 | |
| 1412 | AGTAttctatggtgtATAC | VTCN1\|PDCD1LG2 | |
| 1413 | AGtattctatggtgtATAC | VTCN1\|PDCD1LG2 | |
| 1414 | TAgtattctatggtgtaTAC | VTCN1\|PDCD1LG2 | |
| 1415 | TAgtattctatggtgtatAC | VTCN1\|PDCD1LG2 | |
| 1416 | TAGTattctatggtgtaTAC | VTCN1\|PDCD1LG2 | |
| 1417 | TAtggtgtatacgtgccAC | VTCN1\|PDCD1LG2 | |
| 1418 | TATggtgtatacgtgccAC | VTCN1\|PDCD1LG2 | |
| 1419 | TAtggtgtatacgtgcCAC | VTCN1\|PDCD1LG2 | |
| 1420 | CTAtggtgtatacgtgccAC | VTCN1\|PDCD1LG2 | |
| 1421 | CTatggtgtatacgtgccAC | VTCN1\|PDCD1LG2 | |
| 1422 | CTatggtgtatacgtgcCAC | VTCN1\|PDCD1LG2 | |
| 1423 | ACTAtgcatttGTTA | VTCN1\|PDCD1LG2 | |
| 1424 | ACTAtgcatttgTTA | VTCN1\|PDCD1LG2 | |
| 1425 | ACTatgcatttGTTA | VTCN1\|PDCD1LG2 | |
| 1426 | AAAttcagaaaaCATA | VTCN1\|PDCD1LG2 | |
| 1427 | AAATtcagaaaaCATA | VTCN1\|PDCD1LG2 | |
| 1428 | AAattcagaaaaCATA | VTCN1\|PDCD1LG2 | |
| 1429 | TAtggtgtatacgtgCCA | VTCN1\|PDCD1LG2 | |
| 1430 | TATggtgtatacgtgcCA | VTCN1\|PDCD1LG2 | |
| 1431 | TAtggtgtatacgtgcCA | VTCN1\|PDCD1LG2 | |
| 1432 | CTAtggtgtatacgtgcCA | VTCN1\|PDCD1LG2 | |
| 1433 | CTatggtgtatacgtgcCA | VTCN1\|PDCD1LG2 | |
| 1434 | CTatggtgtatacgtgCCA | VTCN1\|PDCD1LG2 | |
| 1435 | TCtatggtgtatacgtgcCA | VTCN1\|PDCD1LG2 | |
| 1436 | TCtatggtgtatacgtgCCA | VTCN1\|PDCD1LG2 | |
| 1437 | TCTatggtgtatacgtgcCA | VTCN1\|PDCD1LG2 | |
| 1438 | ACctgagtgtTACA | VTCN1\|PDCD1LG2 | |
| 1439 | ACCTgagtgtTACA | VTCN1\|PDCD1LG2 | |
| 1440 | ACCtgagtgtTACA | VTCN1\|PDCD1LG2 | |
| 1441 | TATggtgtatacgtgccaCA | VTCN1\|PDCD1LG2 | |
| 1442 | TAtggtgtatacgtgccaCA | VTCN1\|PDCD1LG2 | |
| 1443 | TAtggtgtatacgtgccACA | VTCN1\|PDCD1LG2 | |
| 1444 | ACTAtgcatttgtTAA | VTCN1\|PDCD1LG2 | |
| 1445 | ACTAtgcatttgTTAA | VTCN1\|PDCD1LG2 | |
| 1446 | ACTatgcatttgTTAA | VTCN1\|PDCD1LG2 | |
| 1447 | CTATgcatttgtTAAA | VTCN1\|PDCD1LG2 | |
| 1448 | CTATgcatttgttAAA | VTCN1\|PDCD1LG2 | |
| 1449 | CTAtgcatttgtTAAA | VTCN1\|PDCD1LG2 | |
| 1450 | ACTAtgcatttgttAAA | VTCN1\|PDCD1LG2 | |
| 1451 | ACTatgcatttgtTAAA | VTCN1\|PDCD1LG2 | |
| 1452 | ACTAtgcatttgtTAAA | VTCN1\|PDCD1LG2 | |
| 1453 | CTATgcatttgttaaAA | VTCN1\|PDCD1LG2 | |
| 1454 | CTATgcatttgttaAAA | VTCN1\|PDCD1LG2 | |
| 1455 | CTATgcatttgttAAAA | VTCN1\|PDCD1LG2 | |
| 1456 | ACTAtgcatttgttaaAA | VTCN1\|PDCD1LG2 | |
| 1457 | ACTAtgcatttgttaAAA | VTCN1\|PDCD1LG2 | |
| 1458 | ACTAtgcatttgttAAAA | VTCN1\|PDCD1LG2 | |
| 1459 | AAATggagcaatTGTAC | IDO1\|PDCD1LG2 | CRM0140 |
| 1460 | ACTGaggaatacggaAG | CD274\|CTLA4 | CRM0141 |
| 1461 | AGTttggcgacaaAATT | CD274\|PDCD1LG2 | CRM0142 |
| 1462 | CAggaacactagaggGT | PDCD1\|PDCD1LG2 | CRM0143 |
| 1463 | CATgaggaatacgGAAG | CD274\|CTLA4 | CRM0144 |
| 1464 | CCtacagggaaagtGAA | CD274\|PDCD1LG2 | CRM0145 |
| 1465 | CCTtaagatacTGTT | CD274\|IDO1\| | CRM0146 |
| | | PDCD1LG2 | |
| 1466 | CTgcacgtccagcCC | IDO1\|PDCD1\| | CRM0147 |
| | | PDCD1LG2 | |
| 1467 | CTTtggttgattttgTTG | CD274\|PDCD1LG2 | CRM0148 |
| 1468 | GAcagtgcatctagCT | IDO1\|PDCD1LG2 | CRM0149 |
| 1469 | GAggaatacggaagTCA | CD274\|CTLA4 | CRM0150 |
| 1470 | GTgggttgatgagagAG | PDCD1\|PDCD1LG2 | CRM0151 |
| 1471 | TCtgcgaggtagatgTT | IDO1\|PDCD1LG2 | CRM0152 |
| 1472 | TGaggaatacggaagCC | CD274\|CTLA4 | CRM0153 |

TABLE 3.2

| SEQ ID NO | Oligonucleotide (5′-3′) | targets | oligoID |
|---|---|---|---|
| 2002 | CCctttccttttctttTT | VSIR\|PDCD1LG2 | |
| 2003 | CCctttccttttcttTTT | VSIR\|PDCD1LG2 | |
| 2004 | CCctttccttttctTTTT | VSIR\|PDCD1LG2 | |
| 2005 | GCtgtcaccttgattTT | HAVCR2\|KIR2DL1\|KIR2DL3 | |
| 2006 | GCtgtcaccttgatTTT | HAVCR2\|KIR2DL1\|KIR2DL3 | |
| 2007 | GCTgtcaccttgatTTT | HAVCR2\|KIR2DL1\|KIR2DL3 | |
| 2008 | GAttctgagggcTTT | KIR2DL3\|TIGIT | |
| 2009 | GAttctgagggCTTT | KIR2DL3\|TIGIT | |
| 2010 | GATTctgagggCTTT | KIR2DL3\|TIGIT | |
| 2011 | GGattctgagggctTT | KIR2DL3\|TIGIT | |
| 2012 | GGAttctgagggctTT | KIR2DL3\|TIGIT | |
| 2013 | GGATtctgagggcTTT | KIR2DL3\|TIGIT | |
| 2014 | ACTGatacatccTTT | CD274\|VSIR | |
| 2015 | ACTgatacatcCTTT | CD274\|VSIR | |
| 2016 | ACTGatacatcCTTT | CD274\|VSIR | |
| 2017 | AACtgatacatcCTTT | CD274\|VSIR | |
| 2018 | AACTgatacatccTTT | CD274\|VSIR | |
| 2019 | AACTgatacatcCTTT | CD274\|VSIR | |
| 2020 | CACttcactcacTTT | VTCN1\|VSIR | |
| 2021 | CACTtcactcacTTT | VTCN1\|VSIR | |
| 2022 | CACTtcactcaCTTT | VTCN1\|VSIR | |
| 2023 | CCacttcactcactTT | VTCN1\|VSIR | |
| 2024 | CCActtcactcacTTT | VTCN1\|VSIR | |
| 2025 | CCActtcactcaCTTT | VTCN1\|VSIR | |
| 2026 | ACctgctgcaggT | NT5E\|VTCN1 | |
| 2027 | ACCtgctgcaggTT | NT5E\|VTCN1 | |
| 2028 | ACCtgctgcagGTT | NT5E\|VTCN1 | |
| 2029 | CAGagttgttttCTT | NT5E\|PDCD1LG2 | |
| 2030 | CAGagttgtttTCTT | NT5E\|PDCD1LG2 | |
| 2031 | CAGAgttgtttTCTT | NT5E\|PDCD1LG2 | |
| 2032 | GAttctgagggCTT | NT5E\|KIR2DL3\|TIGIT | |
| 2033 | GAttctgaggGCTT | NT5E\|KIR2DL3\|TIGIT | |
| 2034 | GATTctgaggGCTT | NT5E\|KIR2DL3\|TIGIT | |
| 2035 | GGattctgagggcTT | KIR2DL3\|TIGIT | |
| 2036 | GGattctgagggcTT | KIR2DL3\|TIGIT | |
| 2037 | GGATtctgagggCTT | KIR2DL3\|TIGIT | |
| 2038 | GCagagcctcttccTT | PDCD1\|KIR2DL3 | |
| 2039 | GCagagcctcttcCTT | PDCD1\|KIR2DL3 | |
| 2040 | GCAgagcctcttccTT | PDCD1\|KIR2DL3 | |
| 2041 | ACTGatacatcCTT | CD274\|VSIR | |
| 2042 | ACTgatacatCCTT | CD274\|VSIR | |
| 2043 | ACTGatacatCCTT | CD274\|VSIR | |
| 2044 | AACTgatacatcCTT | CD274\|VSIR | |
| 2045 | AACtgatacatCCTT | CD274\|VSIR | |
| 2046 | AACTgatacatCCTT | CD274\|VSIR | |
| 2047 | ACacctctgccccTT | NT5E\|VSIR | |
| 2048 | ACAcctctgccccTT | NT5E\|VSIR | |
| 2049 | ACacctctgcccCTT | NT5E\|VSIR | |
| 2050 | GGtcctgggccccTT | NT5E\|CD276 | |
| 2051 | GGtcctgggcccCTT | NT5E\|CD276 | |
| 2052 | GGTcctgggccccTT | NT5E\|CD276 | |
| 2053 | TGgtcctgggccccTT | NT5E\|CD276 | |
| 2054 | TGGtcctgggccccTT | NT5E\|CD276 | |
| 2055 | TGgtcctgggcccCTT | NT5E\|CD276 | |
| 2056 | GTggtcctgggccccTT | NT5E\|CD276 | |
| 2057 | GTggtcctgggcccCTT | NT5E\|CD276 | |
| 2058 | GTGgtcctgggccccTT | NT5E\|CD276 | |
| 2059 | CCacttcactcacTT | VTCN1\|VSIR | |
| 2060 | CCACttcactcacTT | VTCN1\|VSIR | |
| 2061 | CCACttcactcACTT | VTCN1\|VSIR | |
| 2062 | ATaattctttgtTATT | VTCN1\|TIGIT | |
| 2063 | ATAattctttgtTATT | VTCN1\|TIGIT | |
| 2064 | ATAAttctttgtTATT | VTCN1\|TIGIT | |
| 2065 | ATTCattacacatATT | NT5E\|PDCD1LG2 | |
| 2066 | ATTcattacacaTATT | NT5E\|PDCD1LG2 | |
| 2067 | ATTCattacacaTATT | NT5E\|PDCD1LG2 | |
| 2068 | TCCagggaaaaGATT | HAVCR2\|TDO2 | |
| 2069 | TCCAgggaaaagATT | HAVCR2\|TDO2 | |
| 2070 | TCCAgggaaaaGATT | HAVCR2\|TDO2 | |
| 2071 | TTCcagggaaaaGATT | HAVCR2\|TDO2 | |
| 2072 | TTCCagggaaaagATT | HAVCR2\|TDO2 | |
| 2073 | TTCCagggaaaaGATT | HAVCR2\|TDO2 | |
| 2074 | GTaactttatCATT | NT5E\|CD274 | |
| 2075 | GTAactttatCATT | NT5E\|CD274 | |
| 2076 | GTAActttatCATT | NT5E\|CD274 | |
| 2077 | GTAtttttatgAATT | VTCN1\|TDO2 | |
| 2078 | GTATttttatgaATT | VTCN1\|TDO2 | |
| 2079 | GTATttttatgAATT | VTCN1\|TDO2 | |
| 2080 | CCatgctcctatGT | NT5E\|CD276 | |
| 2081 | CCatgctcctaTGT | NT5E\|CD276 | |
| 2082 | CCAtgctcctATGT | NT5E\|CD276 | |
| 2083 | GCcatgctcctatGT | NT5E\|CD276 | |
| 2084 | GCcatgctcctaTGT | NT5E\|CD276 | |
| 2085 | GCCatgctcctatGT | NT5E\|CD276 | |
| 2086 | TGccatgctcctatGT | NT5E\|CD276 | |
| 2087 | TGccatgctcctaTGT | NT5E\|CD276 | |
| 2088 | TGCcatgctcctatGT | NT5E\|CD276 | |
| 2089 | CTtcagttgctgGT | PDCD1LG2\|TIGIT | |
| 2090 | CTTcagttgctGGT | PDCD1LG2\|TIGIT | |
| 2091 | CTTCagttgcTGGT | PDCD1LG2\|TIGIT | |
| 2092 | GGccttggactgGT | CD86\|TIGIT | |
| 2093 | GGccttggactGGT | CD86\|TIGIT | |
| 2094 | GGCcttggactgGT | CD86\|TIGIT | |
| 2095 | TTCaaagtcatTTCT | NT5E\|CD86 | |
| 2096 | TTCAaagtcattTCT | NT5E\|CD86 | |
| 2097 | TTCAaagtcatTTCT | NT5E\|CD86 | |
| 2098 | CAaaatgcatgTTCT | CEACAM1\|VSIR | |
| 2099 | CAAaatgcatgTTCT | CEACAM1\|VSIR | |
| 2100 | CAAAatgcatgTTCT | CEACAM1\|VSIR | |
| 2101 | CCaaaatgcatgtTCT | CEACAM1\|VSIR | |
| 2102 | CCAaaatgcatgtTCT | CEACAM1\|VSIR | |
| 2103 | CCAAaatgcatgTTCT | CEACAM1\|VSIR | |
| 2104 | TCatccgtgtgtCT | NT5E\|CD86 | |
| 2105 | TCATccgtgtgtCT | NT5E\|CD86 | |
| 2106 | TCATccgtgtGTCT | NT5E\|CD86 | |
| 2107 | GGcctgtgccgtCT | VSIR\|LGALS9 | |
| 2108 | GGcctgtgccgTCT | VSIR\|LGALS9 | |
| 2109 | GGCctgtgccgtCT | VSIR\|LGALS9 | |
| 2110 | TGgcctgtgccgtCT | VSIR\|LGALS9 | |
| 2111 | TGgcctgtgccgTCT | VSIR\|LGALS9 | |
| 2112 | TGGcctgtgccgtCT | VSIR\|LGALS9 | |
| 2113 | CTttcttccttttctCT | CEACAM1\|NT5E | |
| 2114 | CTTTcttccttttctCT | CEACAM1\|NT5E | |
| 2115 | CTTTcttccttttcTCT | CEACAM1\|NT5E | |
| 2116 | GCttggacatctCT | CD86\|VSIR | |
| 2117 | GCTTggacatctCT | CD86\|VSIR | |
| 2118 | GCTTggacatCTCT | CD86\|VSIR | |
| 2119 | TATcttctctttgCT | HAVCR2\|PDCD1LG2 | |
| 2120 | TAtcttctcttTGCT | HAVCR2\|PDCD1LG2 | |
| 2121 | TATCttctcttTGCT | HAVCR2\|PDCD1LG2 | |
| 2122 | ATcacacccatggCT | NT5E\|TNFRSF14 | |
| 2123 | ATCacacccatggCT | NT5E\|TNFRSF14 | |
| 2124 | ATCAcacccatggCT | NT5E\|TNFRSF14 | |
| 2125 | CAtcacacccatggCT | NT5E\|TNFRSF14 | |
| 2126 | CATcacacccatggCT | NT5E\|TNFRSF14 | |
| 2127 | CAtcacacccatgGCT | NT5E\|TNFRSF14 | |
| 2128 | TCatcacacccatggCT | NT5E\|TNFRSF14 | |
| 2129 | TCAtcacacccatggCT | NT5E\|TNFRSF14 | |
| 2130 | TCatcacacccatgGCT | NT5E\|TNFRSF14 | |
| 2131 | TCtgggctgtgggCT | VTCN1\|VSIR | |
| 2132 | TCTgggctgtgggCT | VTCN1\|VSIR | |
| 2133 | TCtgggctgtggGCT | VTCN1\|VSIR | |
| 2134 | ATgtcataggaGCT | KIR2DL1\|KIR2DL3\|TDO2 | |
| 2135 | ATGtcataggAGCT | KIR2DL1\|KIR2DL3\|TDO2 | |
| 2136 | ATGTcataggAGCT | KIR2DL1\|KIR2DL3\|TDO2 | |
| 2137 | TGcctgtgaggagCT | PDCD1LG2\|TDO2 | |
| 2138 | TGcctgtgaggaGCT | PDCD1LG2\|TDO2 | |
| 2139 | TGCctgtgaggagCT | PDCD1LG2\|TDO2 | |
| 2140 | TTgcctgtgaggagCT | PDCD1LG2\|TDO2 | CRM0284 |
| 2141 | TTGcctgtgaggagCT | PDCD1LG2\|TDO2 | |
| 2142 | TTgcctgtgaggaGCT | PDCD1LG2\|TDO2 | |
| 2143 | AGcatcagatttcCT | HAVCR2\|CD276 | |
| 2144 | AGcatcagatttCCT | HAVCR2\|CD276 | |
| 2145 | AGCatcagattTCCT | HAVCR2\|CD276 | |
| 2146 | ATccaattttaTCCT | NT5E\|CD86 | |
| 2147 | ATCcaattttaTCCT | NT5E\|CD86 | |
| 2148 | ATCCaattttaTCCT | NT5E\|CD86 | |
| 2149 | AACtgatacaTCCT | CD274\|VSIR | |
| 2150 | AACTgatacatCCT | CD274\|VSIR | |
| 2151 | AACTgatacaTCCT | CD274\|VSIR | |
| 2152 | GCctccagctctgcCT | NT5E\|PDCD1 | |
| 2153 | GCCtccagctctgcCT | NT5E\|PDCD1 | |
| 2154 | GCctccagctctgCCT | NT5E\|PDCD1 | |
| 2155 | TGgtcctgggcccCT | NT5E\|CD276 | |
| 2156 | TGGtcctgggcccCT | NT5E\|CD276 | |
| 2157 | TGgtcctgggccCCT | NT5E\|CD276 | |
| 2158 | GTggtcctgggcccCT | NT5E\|CD276 | |
| 2159 | GTGgtcctgggcccCT | NT5E\|CD276 | |
| 2160 | GTggtcctgggccCCT | NT5E\|CD276 | |
| 2161 | GGcaggagccccCT | LAG3\|CD276 | |
| 2162 | GGcaggagcccCCT | LAG3\|CD276 | |
| 2163 | GGCaggagccccCT | LAG3\|CD276 | |
| 2164 | TATgtaaccccaCT | VTCN1\|VSIR | |
| 2165 | TATGtaaccccACT | VTCN1\|VSIR | |
| 2166 | TATGtaacccCACT | VTCN1\|VSIR | |
| 2167 | TTatgtaaccccACT | VTCN1\|VSIR | |
| 2168 | TTATgtaaccccaCT | VTCN1\|VSIR | |
| 2169 | TTATgtaacccCACT | VTCN1\|VSIR | |
| 2170 | GTtatgtaaccccaCT | VTCN1\|VSIR | |
| 2171 | GTTatgtaaccccaCT | VTCN1\|VSIR | |
| 2172 | GTTAtgtaaccccACT | VTCN1\|VSIR | |
| 2173 | AGttatgtaaccccaCT | VTCN1\|VSIR | |
| 2174 | AGTtatgtaaccccaCT | VTCN1\|VSIR | |
| 2175 | AGttatgtaacccCACT | VTCN1\|VSIR | |
| 2176 | CAgttatgtaaccccaCT | VTCN1\|VSIR | |
| 2177 | CAgttatgtaaccccACT | VTCN1\|VSIR | |
| 2178 | CAGttatgtaaccccaCT | VTCN1\|VSIR | |
| 2179 | CCtgtgactacACT | CD86\|KIR2DL1 | |
| 2180 | CCTGtgactacaCT | CD86\|KIR2DL1 | |
| 2181 | CCTGtgactaCACT | CD86\|KIR2DL1 | |
| 2182 | GGAAtacttcaaACT | VTCN1\|TDO2 | |
| 2183 | GGAatacttcaAACT | VTCN1\|TDO2 | |
| 2184 | GGAAtacttcaAACT | VTCN1\|TDO2 | |
| 2185 | TACattattttgtTTAT | NT5E\|VTCN1 | |
| 2186 | TACAttattttgttTAT | NT5E\|VTCN1 | |
| 2187 | TACAttattttgtTTAT | NT5E\|VTCN1 | |
| 2188 | CGGcaaacatTTAT | NT5E\|VTCN1 | |
| 2189 | CGGCaaacattTAT | NT5E\|VTCN1 | |
| 2190 | CGGCaaacatTTAT | NT5E\|VTCN1 | |
| 2191 | TGccatgctcctAT | NT5E\|CD276 | |
| 2192 | TGCcatgctcctAT | NT5E\|CD276 | |
| 2193 | TGCCatgctcctAT | NT5E\|CD276 | |
| 2194 | TCAAttgatcaTAT | PDCD1LG2\|TDO2 | |
| 2195 | TCAattgatcATAT | PDCD1LG2\|TDO2 | |
| 2196 | TCAAttgatcATAT | PDCD1LG2\|TDO2 | |
| 2197 | ATCaattgatcATAT | PDCD1LG2\|TDO2 | |
| 2198 | ATCAattgatcaTAT | PDCD1LG2\|TDO2 | |
| 2199 | ATCAattgatcATAT | PDCD1LG2\|TDO2 | |
| 2200 | ATTcattacacATAT | NT5E\|PDCD1LG2 | |
| 2201 | ATTCattacacaTAT | NT5E\|PDCD1LG2 | |
| 2202 | ATTCattacacATAT | NT5E\|PDCD1LG2 | |
| 2203 | TTTGtatattgGAT | NT5E\|CD80 | |
| 2204 | TTTgtatattGGAT | NT5E\|CD80 | |
| 2205 | TTTGtatattGGAT | NT5E\|CD80 | |
| 2206 | CATGccaagaggAT | VTCN1\|TDO2 | |
| 2207 | CATGccaagagGAT | VTCN1\|TDO2 | |
| 2208 | CATGccaagaGGAT | VTCN1\|TDO2 | |
| 2209 | TTCcagggaaaAGAT | HAVCR2\|TDO2 | |
| 2210 | TTCCagggaaaaGAT | HAVCR2\|TDO2 | |
| 2211 | TTCCagggaaaAGAT | HAVCR2\|TDO2 | |
| 2212 | GAAcctggaggtcAT | NT5E\|VTCN1 | |
| 2213 | GAAcctggaggtCAT | NT5E\|VTCN1 | |
| 2214 | GAACctggaggTCAT | NT5E\|VTCN1 | |
| 2215 | TTActcatacTCAT | CD86\|TDO2 | |
| 2216 | TTACtcatactCAT | CD86\|TDO2 | |
| 2217 | TTACtcatacTCAT | CD86\|TDO2 | |
| 2218 | ATATcttatatcCAT | CD86\|KIR2DL1\|KIR2DL3 | |
| 2219 | ATAtcttatatCCAT | CD86\|KIR2DL1\|KIR2DL3 | |
| 2220 | ATATcttatatCCAT | CD86\|KIR2DL1\|KIR2DL3 | |
| 2221 | ACcccctccccacAT | CEACAM1\|CD80 | |
| 2222 | ACcccctccccaCAT | CEACAM1\|CD80 | |
| 2223 | ACCccctccccacAT | CEACAM1\|CD80 | |
| 2224 | AAccccctccccacAT | CEACAM1\|CD80 | |
| 2225 | AACcccctccccacAT | CEACAM1\|CD80 | |
| 2226 | AAccccctccccaCAT | CEACAM1\|CD80 | |
| 2227 | TGGGaaatgggtAAT | KIR2DL3\|CD274 | |
| 2228 | TGGgaaatgggTAAT | KIR2DL3\|CD274 | |
| 2229 | TGGGaaatgggTAAT | KIR2DL3\|CD274 | |
| 2230 | CTGggaaatgggtAAT | KIR2DL3\|CD274 | |
| 2231 | CTGGgaaatgggtAAT | KIR2DL3\|CD274 | |
| 2232 | CTGGgaaatgggTAAT | KIR2DL3\|CD274 | |
| 2233 | ACTCtctagagAAT | VTCN1\|VSIR | |
| 2234 | ACTctctagaGAAT | VTCN1\|VSIR | |
| 2235 | ACTCtctagaGAAT | VTCN1\|VSIR | |
| 2236 | TTttccttgtaCAAT | NT5E\|CD80 | |
| 2237 | TTTtccttgtaCAAT | NT5E\|CD80 | |
| 2238 | TTTTccttgtaCAAT | NT5E\|CD80 | |
| 2239 | ATtttccttgtaCAAT | NT5E\|CD80 | |
| 2240 | ATTttccttgtaCAAT | NT5E\|CD80 | |
| 2241 | ATTTtccttgtaCAAT | NT5E\|CD80 | |
| 2242 | AAttttccttgtaCAAT | NT5E\|CD80 | |
| 2243 | AATtttccttgtaCAAT | NT5E\|CD80 | |
| 2244 | AATTttccttgtaCAAT | NT5E\|CD80 | |
| 2245 | TAATtttccttgtacAAT | NT5E\|CD80 | |
| 2246 | TAAttttccttgtaCAAT | NT5E\|CD80 | |
| 2247 | TAATtttccttgtaCAAT | NT5E\|CD80 | |
| 2248 | CTaattttccttgtacaAT | NT5E\|CD80 | |
| 2249 | CTAAttttccttgtacAAT | NT5E\|CD80 | |
| 2250 | CTAAttttccttgtaCAAT | NT5E\|CD80 | |
| 2251 | TCTcttgcctcaaAT | KIR2DL1\|PDCD1LG2 | |
| 2252 | TCTCttgcctcaaAT | KIR2DL1\|PDCD1LG2 | |
| 2253 | TCTCttgcctcAAAT | KIR2DL1\|PDCD1LG2 | |
| 2254 | ATATacatttacaaAT | HAVCR2\|VTCNl | |
| 2255 | ATATacatttacaAAT | HAVCR2\|VTCNl | |
| 2256 | ATATacatttacAAAT | HAVCR2\|VTCNl | |
| 2257 | ACCttagacaTTTG | CD80\|KIR2DL1\|KIR2DL3 | |
| 2258 | ACCTtagacatTTG | CD80\|KIR2DL1\|KIR2DL3 | |
| 2259 | ACCTtagacaTTTG | CD80\|KIR2DL1\|KIR2DL3 | |
| 2260 | TTgacctcagctcTG | CEACAM1\|CD86 | |
| 2261 | TTGacctcagctCTG | CEACAM1\|CD86 | |
| 2262 | TTGAcctcagctCTG | CEACAM1\|CD86 | |
| 2263 | TTCtttctgtggcTG | HAVCR2\|VTCNl | |
| 2264 | TTCtttctgtggCTG | HAVCR2\|VTCNl | |
| 2265 | TTCtttctgtgGCTG | HAVCR2\|VTCNl | |
| 2266 | ACAatctagcccTG | CEACAM1\|NT5E | |
| 2267 | ACAAtctagccCTG | CEACAM1\|NT5E | |
| 2268 | ACAAtctagcCCTG | CEACAM1\|NT5E | |
| 2269 | CATTattttgtttATG | NT5E\|VTCNl | |
| 2270 | CATtattttgttTATG | NT5E\|VTCNl | |
| 2271 | CATTattttgttTATG | NT5E\|VTCNl | |
| 2272 | ACATtattttgtttATG | NT5E\|VTCN1 | |
| 2273 | ACAttattttgttTATG | NT5E\|VTCN1 | |
| 2274 | ACATtattttgttTATG | NT5E\|VTCN1 | |
| 2275 | TACAttattttgtttATG | NT5E\|VTCN1 | |
| 2276 | TACattattttgttTATG | NT5E\|VTCN1 | |
| 2277 | TACAttattttgttTATG | NT5E\|VTCN1 | |
| 2278 | GCcatgctcctaTG | NT5E\|CD276 | |
| 2279 | GCCatgctcctaTG | NT5E\|CD276 | |
| 2280 | GCCAtgctcctaTG | NT5E\|CD276 | |
| 2281 | TGccatgctcctaTG | NT5E\|CD276 | |
| 2282 | TGccatgctcctATG | NT5E\|CD276 | |
| 2283 | TGCcatgctcctATG | NT5E\|CD276 | |
| 2284 | TCAtcacacccaTG | NT5E\|TNFRSF14 | |
| 2285 | TCATcacacccATG | NT5E\|TNFRSF14 | |
| 2286 | TCATcacaccCATG | NT5E\|TNFRSF14 | |
| 2287 | TTTtaatgtttTTGG | NT5E\|HAVCR2 | |
| 2288 | TTTTaatgttttTGG | NT5E\|HAVCR2 | |
| 2289 | TTTTaatgtttTTGG | NT5E\|HAVCR2 | |
| 2290 | TTCtgagggcttGG | NT5E\|VSIR | |
| 2291 | TTCtgagggctTGG | NT5E\|VSIR | |
| 2292 | TTCTgagggcTTGG | NT5E\|VSIR | |
| 2293 | GGtgtgtgtgggtgtGG | CD86\|VSIR | |
| 2294 | GGTgtgtgtgggtgtGG | CD86\|VSIR | |
| 2295 | GGtgtgtgtgggtgTGG | CD86\|VSIR | |
| 2296 | ATattgggccctGG | CEACAM1\|KIR2DL3 | |
| 2297 | ATATtgggccctGG | CEACAM1\|KIR2DL3 | |
| 2298 | ATAttgggccCTGG | CEACAM1\|KIR2DL3 | |
| 2299 | CAtcacacccatGG | NT5E\|TNFRSF14 | |
| 2300 | CATCacacccatGG | NT5E\|TNFRSF14 | |
| 2301 | CATCacacccATGG | NT5E\|TNFRSF14 | |
| 2302 | TCatcacacccatGG | NT5E\|TNFRSF14 | |
| 2303 | TCatcacacccATGG | NT5E\|TNFRSF14 | |
| 2304 | TCATcacacccaTGG | NT5E\|TNFRSF14 | |
| 2305 | TTaatgttttTGGG | NT5E\|HAVCR2 | |
| 2306 | TTAatgttttTGGG | NT5E\|HAVCR2 | |
| 2307 | TTAAtgttttTGGG | NT5E\|HAVCR2 | |
| 2308 | TTTAatgtttttGGG | NT5E\|HAVCR2 | |
| 2309 | TTTaatgttttTGGG | NT5E\|HAVCR2 | |
| 2310 | TTTAatgttttTGGG | NT5E\|HAVCR2 | |
| 2311 | TTTTaatgtttttGGG | NT5E\|HAVCR2 | |
| 2312 | TTTtaatgttttTGGG | NT5E\|HAVCR2 | |
| 2313 | TTTTaatgttttTGGG | NT5E\|HAVCR2 | |
| 2314 | TGtgtgtccaagGG | NT5E\|TIGIT | |
| 2315 | TGTGtgtccaagGG | NT5E\|TIGIT | |
| 2316 | TGTgtgtccaAGGG | NT5E\|TIGIT | |
| 2317 | CCagctggacgcGG | LAG3\|HMOX1 | |
| 2318 | CCAgctggacgcGG | LAG3\|HMOX1 | |
| 2319 | CCagctggacgCGG | LAG3\|HMOX1 | |
| 2320 | CCacccactcagaGG | NT5E\|CD276 | |
| 2321 | CCacccactcagAGG | NT5E\|CD276 | |
| 2322 | CCAcccactcagaGG | NT5E\|CD276 | |
| 2323 | AGgctgctaccaGG | CD80\|TIGIT | |
| 2324 | AGgctgctaccAGG | CD80\|TIGIT | |
| 2325 | AGGctgctaccAGG | CD80\|TIGIT | |
| 2326 | AGtgcccacatcCG | CEACAM1\|TNFRSF14 | |
| 2327 | AGTgcccacatcCG | CEACAM1\|TNFRSF14 | |
| 2328 | AGtgcccacatCCG | CEACAM1\|TNFRSF14 | |
| 2329 | TTGTgtttggtgAG | CEACAM1\|CD86 | |
| 2330 | TTGtgtttggTGAG | CEACAM1\|CD86 | |
| 2331 | TTGTgtttggTGAG | CEACAM1\|CD86 | |
| 2332 | CTTgtgtttggtgAG | CEACAM1\|CD86 | |
| 2333 | CTtgtgtttggTGAG | CEACAM1\|CD86 | |
| 2334 | CTTGtgtttggTGAG | CEACAM1\|CD86 | |
| 2335 | TCtatttttaattttctGAG | CD80\|CD86 | |
| 2336 | TCtatttttaattttcTGAG | CD80\|CD86 | |
| 2337 | TCTAtttttaattttcTGAG | CD80\|CD86 | |
| 2338 | GTcagcctcactgAG | CEACAM1\|VSIR | |
| 2339 | GTcagcctcactGAG | CEACAM1\|VSIR | |
| 2340 | GTcagcctcacTGAG | CEACAM1\|VSIR | |
| 2341 | AGcaaccagagGAG | NT5E\|CD86 | |
| 2342 | AGcaaccagaGGAG | NT5E\|CD86 | |
| 2343 | AGCaaccagaGGAG | NT5E\|CD86 | |
| 2344 | AGggccagacaggAG | CD276\|VSIR | |
| 2345 | AGggccagacagGAG | CD276\|VSIR | |
| 2346 | AGGgccagacaggAG | CD276\|VSIR | |
| 2347 | CTcaccctgagtcAG | CD86\|VSIR | |
| 2348 | CTcaccctgagtCAG | CD86\|VSIR | |
| 2349 | CTcaccctgagTCAG | CD86\|VSIR | |
| 2350 | TCtcaccctgagtcAG | CD86\|VSIR | |
| 2351 | TCTcaccctgagtcAG | CD86\|VSIR | |
| 2352 | TCTCaccctgagtcAG | CD86\|VSIR | |
| 2353 | GAtgaggaaacagactcAG | CD274\|VSIR | |
| 2354 | GATGaggaaacagactcAG | CD274\|VSIR | |
| 2355 | GATgaggaaacagacTCAG | CD274\|VSIR | |
| 2356 | AGatgaggaaacagactcAG | CD274\|VSIR | |
| 2357 | AGAtgaggaaacagactcAG | CD274\|VSIR | |
| 2358 | AGAtgaggaaacagactCAG | CD274\|VSIR | |
| 2359 | AAGcaaatgtctgCAG | CEACAM1\|PDCD1LG2 | |
| 2360 | AAGcaaatgtctGCAG | CEACAM1\|PDCD1LG2 | |
| 2361 | AAGCaaatgtctGCAG | CEACAM1\|PDCD1LG2 | |
| 2362 | ATAggataatGCAG | CEACAM1\|CD276 | |
| 2363 | ATAGgataatgCAG | CEACAM1\|CD276 | |
| 2364 | ATAGgataatGCAG | CEACAM1\|CD276 | |
| 2365 | GGctggtgttggcAG | VSIR\|PDCD1LG2 | |
| 2366 | GGctggtgttggCAG | VSIR\|PDCD1LG2 | |
| 2367 | GGctggtgttggcAG | VSIR\|PDCD1LG2 | |
| 2368 | TGgctggtgttggcAG | VSIR\|PDCD1LG2 | |
| 2369 | TGGctggtgttggcAG | VSIR\|PDCD1LG2 | |
| 2370 | TGgctggtgttggCAG | VSIR\|PDCD1LG2 | |
| 2371 | GTggctggtgttggcAG | VSIR\|PDCD1LG2 | |
| 2372 | GTGgctggtgttggcAG | VSIR\|PDCD1LG2 | |
| 2373 | GTggctggtgttggcAG | VSIR\|PDCD1LG2 | |
| 2374 | TCtgctacttcccAG | CD80\|VSIR | |
| 2375 | TCTgctacttcccAG | CD80\|VSIR | |
| 2376 | TCTgctacttccCAG | CD80\|VSIR | |
| 2377 | CTctgctacttcccAG | CD80\|VSIR | |
| 2378 | CTCtgctacttcccAG | CD80\|VSIR | |
| 2379 | CTctgctacttccCAG | CD80\|VSIR | |
| 2380 | CCtctgctacttcccAG | CD80\|VSIR | |
| 2381 | CCTctgctacttcccAG | CD80\|VSIR | |
| 2382 | CCtctgctacttccCAG | CD80\|VSIR | |
| 2383 | TCTCcaagcaagaAG | VSIR\|IDO1 | |
| 2384 | TCTccaagcaaGAAG | VSIR\|IDOl | |
| 2385 | TCTCcaagcaaGAAG | VSIR\|IDOl | |
| 2386 | TCTCcaagcaagAAG | VSIR\|IDOl | CRM0285 |
| 2387 | TGCTttccaacaAG | NT5E\|LGALS9 | |
| 2388 | TGCtttccaaCAAG | NT5E\|LGALS9 | |
| 2389 | TGCTttccaaCAAG | NT5E\|LGALS9 | |
| 2390 | ATTctgagggctTTC | KIR2DL3\|TIGIT | |
| 2391 | ATTCtgagggctTTC | KIR2DL3\|TIGIT | |
| 2392 | ATTCtgagggcTTTC | KIR2DL3\|TIGIT | |
| 2393 | GAttctgagggcttTC | KIR2DL3\|TIGIT | |
| 2394 | GATtctgagggctTTC | KIR2DL3\|TIGIT | |
| 2395 | GATTctgagggcTTTC | KIR2DL3\|TIGIT | |
| 2396 | GGattctgagggcttTC | KIR2DL3\|TIGIT | |
| 2397 | GGattctgagggctTTC | KIR2DL3\|TIGIT | |
| 2398 | GGAttctgagggctTTC | KIR2DL3\|TIGIT | |
| 2399 | CCAAaatgcatgTTC | CEACAM1\|VSIR | |
| 2400 | CCAaaatgcatGTTC | CEACAM1\|VSIR | |
| 2401 | CCAAaatgcatGTTC | CEACAM1\|VSIR | |
| 2402 | CAaggccagggtTC | NT5E\|TIGIT | |
| 2403 | CAAggccagggTTC | NT5E\|TIGIT | |
| 2404 | CAAGgccagggTTC | NT5E\|TIGIT | |
| 2405 | CAgagcctcttcctTC | PDCD1\|KIR2DL1\|KIR2DL3 | |
| 2406 | CAgagcctcttccTTC | PDCD1\|KIR2DL1\|KIR2DL3 | |
| 2407 | CAGagcctcttcctTC | PDCD1\|KIR2DL1\|KIR2DL3 | |
| 2408 | GCagagcctcttcctTC | PDCD1\|KIR2DL3 | |
| 2409 | GCagagcctcttccTTC | PDCD1\|KIR2DL3 | |
| 2410 | GCAgagcctcttcctTC | PDCD1\|KIR2DL3 | |
| 2411 | CCAcaggaatATTC | NT5E\|CD80 | |
| 2412 | CCACaggaataTTC | NT5E\|CD80 | |
| 2413 | CCACaggaatATTC | NT5E\|CD80 | |
| 2414 | CAtgctcctatGTC | NT5E\|CD276 | |
| 2415 | CAtgctcctaTGTC | NT5E\|CD276 | |
| 2416 | CATGctcctaTGTC | NT5E\|CD276 | |
| 2417 | CCatgctcctatgTC | NT5E\|CD276 | |
| 2418 | CCAtgctcctatgTC | NT5E\|CD276 | |
| 2419 | CCATgctcctatgTC | NT5E\|CD276 | |
| 2420 | GCcatgctcctatgTC | NT5E\|CD276 | |
| 2421 | GCcatgctcctatGTC | NT5E\|CD276 | |
| 2422 | GCCatgctcctatgTC | NT5E\|CD276 | |
| 2423 | TGccatgctcctatgTC | NT5E\|CD276 | |
| 2424 | TGccatgctcctatGTC | NT5E\|CD276 | |
| 2425 | TGCcatgctcctatgTC | NT5E\|CD276 | |
| 2426 | CTgtgttgtgggTC | HAVCR2\|PDCD1LG2 | |
| 2427 | CTGtgttgtggGTC | HAVCR2\|PDCD1LG2 | |
| 2428 | CTGTgttgtggGTC | HAVCR2\|PDCD1LG2 | |
| 2429 | TGgcctgtgccgTC | VSIR\|LGALS9 | |
| 2430 | TGgcctgtgccGTC | VSIR\|LGALS9 | |
| 2431 | TGGcctgtgccgTC | VSIR\|LGALS9 | |
| 2432 | TCtcaccctgagTC | CD86\|VSIR | |
| 2433 | TCTCaccctgagTC | CD86\|VSIR | |
| 2434 | TCTcaccctgAGTC | CD86\|VSIR | |
| 2435 | CAccagccatgtcTC | CD86\|TIGIT | |
| 2436 | CAccagccatgtCTC | CD86\|TIGIT | |
| 2437 | CAccagccatgTCTC | CD86\|TIGIT | |
| 2438 | CTttcttccttttctcTC | CEACAM1\|NT5E | |
| 2439 | CTTtcttccttttctcTC | CEACAM1\|NT5E | |
| 2440 | CTTTcttccttttctcTC | CEACAM1\|NT5E | |
| 2441 | TCcggttcttgcTC | LAG3\|CD80 | |
| 2442 | TCCggttcttgcTC | LAG3\|CD80 | |
| 2443 | TCCGgttcttgcTC | LAG3\|CD80 | |
| 2444 | TCCAattttatccTC | NT5E\|CD86 | |
| 2445 | TCCAattttatcCTC | NT5E\|CD86 | |
| 2446 | TCCAattttatCCTC | NT5E\|CD86 | |
| 2447 | ATCcaattttatcCTC | NT5E\|CD86 | |
| 2448 | ATCcaattttatCCTC | NT5E\|CD86 | |
| 2449 | ATCCaattttatCCTC | NT5E\|CD86 | |
| 2450 | CCtgagagtgccTC | CEACAM1\|VSIR | |
| 2451 | CCtgagagtgcCTC | CEACAM1\|VSIR | |
| 2452 | CCTgagagtgcCTC | CEACAM1\|VSIR | |
| 2453 | GGcctctaccccTC | NT5E\|VTCN1 | |
| 2454 | GGcctctacccCTC | NT5E\|VTCN1 | |
| 2455 | GGCctctaccccTC | NT5E\|VTCN1 | |
| 2456 | GAtgaggaaacagACTC | CD274\|VSIR | |
| 2457 | GATgaggaaacagACTC | CD274\|VSIR | |
| 2458 | GATGaggaaacagACTC | CD274\|VSIR | |
| 2459 | AGatgaggaaacagaCTC | CD274\|VSIR | |
| 2460 | AGATgaggaaacagacTC | CD274\|VSIR | |
| 2461 | AGATgaggaaacagACTC | CD274\|VSIR | |
| 2462 | GCctcagatctATC | PDCD1LG2\|TIGIT | |
| 2463 | GCctcagatctATC | PDCD1LG2\|TIGIT | |
| 2464 | GCCtcagatcTATC | PDCD1LG2\|TIGIT | |
| 2465 | ACTCactgatgATC | NT5E\|VSIR | |
| 2466 | ACTcactgatGATC | NT5E\|VSIR | |
| 2467 | ACTCactgatGATC | NT5E\|VSIR | |
| 2468 | TACTcatactcATC | CD86\|TDO2 | |
| 2469 | TACtcatactCATC | CD86\|TDO2 | |
| 2470 | TACTcatactCATC | CD86\|TDO2 | |
| 2471 | TTACtcatactcATC | CD86\|TDO2 | |
| 2472 | TTActcatactCATC | CD86\|TDO2 | |
| 2473 | TTACtcatactCATC | CD86\|TDO2 | |
| 2474 | TTecccaggccaTC | NT5E\|CD86 | |
| 2475 | TTCcccaggccaTC | NT5E\|CD86 | |
| 2476 | TTCcccaggccATC | NT5E\|CD86 | |
| 2477 | CCctgctgggccctGC | CD276\|TIGIT | |
| 2478 | CCctgctgggcccTGC | CD276\|TIGIT | |
| 2479 | CCCtgctgggccctGC | CD276\|TIGIT | |
| 2480 | CAGGaaaagacTGC | NT5E\|CD276 | |
| 2481 | CAGgaaaagaCTGC | NT5E\|CD276 | |
| 2482 | CAGGaaaagaCTGC | NT5E\|CD276 | |
| 2483 | ATTattttgtttATGC | NT5E\|VTCN1 | |
| 2484 | ATTAttttgtttaTGC | NT5E\|VTCN1 | |
| 2485 | ATTAttttgtttATGC | NT5E\|VTCN1 | |
| 2486 | CATTattttgtttatGC | NT5E\|VTCN1 | |
| 2487 | CATtattttgtttATGC | NT5E\|VTCN1 | |
| 2488 | CATTattttgtttATGC | NT5E\|VTCN1 | |
| 2489 | ACAttattttgtttatGC | NT5E\|VTCN1 | |
| 2490 | ACAttattttgtttaTGC | NT5E\|VTCN1 | |
| 2491 | ACATtattttgtttATGC | NT5E\|VTCN1 | |
| 2492 | TACattattttgtttatGC | NT5E\|VTCN1 | |
| 2493 | TACattattttgtttaTGC | NT5E\|VTCN1 | |
| 2494 | TACAttattttgtttATGC | NT5E\|VTCN1 | |
| 2495 | CCtgcactagatGC | CEACAM1\|VSIR | |
| 2496 | CCtgcactagaTGC | CEACAM1\|VSIR | |
| 2497 | CCTgcactagaTGC | CEACAM1\|VSIR | |
| 2498 | GTggctggtgttgGC | VSIR\|PDCD1LG2 | |
| 2499 | GTggctggtgttGGC | VSIR\|PDCD1LG2 | |
| 2500 | GTGgctggtgttgGC | VSIR\|PDCD1LG2 | |
| 2501 | CTttgccctcctgGC | NT5E\|LGALS9 | |
| 2502 | CTTtgccctcctgGC | NT5E\|LGALS9 | |
| 2503 | CTttgccctcctGGC | NT5E\|LGALS9 | |
| 2504 | ATcacacccatgGC | NT5E\|TNFRSF14 | |
| 2505 | ATCacacccatGGC | NT5E\|TNFRSF14 | |
| 2506 | ATCAcacccatGGC | NT5E\|TNFRSF14 | |
| 2507 | CAtcacacccatgGC | NT5E\|TNFRSF14 | |
| 2508 | CATcacacccatgGC | NT5E\|TNFRSF14 | |
| 2509 | CAtcacacccatGGC | NT5E\|TNFRSF14 | |
| 2510 | TCatcacacccatgGC | NT5E\|TNFRSF14 | |
| 2511 | TCatcacacccatGGC | NT5E\|TNFRSF14 | |
| 2512 | TCAtcacacccatgGC | NT5E\|TNFRSF14 | |
| 2513 | ATGgttgaaatGGC | VSIR\|PDCD1LG2 | |
| 2514 | ATGGttgaaatGGC | VSIR\|PDCD1LG2 | |
| 2515 | ATGGttgaaaTGGC | VSIR\|PDCD1LG2 | |
| 2516 | TGGattaagggAGC | HAVCR2\|TIGIT | |
| 2517 | TGGAttaagggAGC | HAVCR2\|TIGIT | |
| 2518 | TGGAttaaggGAGC | HAVCR2\|TIGIT | |
| 2519 | CTggattaagggaGC | HAVCR2\|TIGIT | |
| 2520 | CTGGattaagggaGC | HAVCR2\|TIGIT | |
| 2521 | CTGgattaaggGAGC | HAVCR2\|TIGIT | |
| 2522 | TTgcctgtgaggaGC | PDCD1LG2\|TDO2 | |
| 2523 | TTgcctgtgaggAGC | PDCD1LG2\|TDO2 | |
| 2524 | TTGcctgtgaggAGC | PDCD1LG2\|TDO2 | |
| 2525 | GGgtagagaaggaGC | LAG3\|HAVCR2 | |
| 2526 | GGgtagagaaggAGC | LAG3\|HAVCR2 | |
| 2527 | GGgtagagaagGAGC | LAG3\|HAVCR2 | |
| 2528 | GGagaggagaagAGC | CD276\|TDO2 | |
| 2529 | GGagaggagaaGAGC | CD276\|TDO2 | |
| 2530 | GGAgaggagaaGAGC | CD276\|TDO2 | |
| 2531 | TAGgataatgcAGC | CEACAM1\|CD276 | |
| 2532 | TAGGataatgcAGC | CEACAM1\|CD276 | |
| 2533 | TAGGataatgCAGC | CEACAM1\|CD276 | |
| 2534 | ATAggataatgcAGC | CEACAM1\|CD276 | |
| 2535 | ATAGgataatgcAGC | CEACAM1\|CD276 | |
| 2536 | ATAGgataatgCAGC | CEACAM1\|CD276 | |
| 2537 | CTgctacttcccaGC | CD80\|VSIR | |
| 2538 | CTgctacttcccAGC | CD80\|VSIR | |
| 2539 | CTGctacttcccaGC | CD80\|VSIR | |
| 2540 | TCtgctacttcccaGC | CD80\|VSIR | |
| 2541 | TCtgctacttcccAGC | CD80\|VSIR | |
| 2542 | TCTgctacttcccaGC | CD80\|VSIR | |
| 2543 | CTctgctacttcccaGC | CD80\|VSIR | |
| 2544 | CTctgctacttcccAGC | CD80\|VSIR | |
| 2545 | CTCtgctacttcccaGC | CD80\|VSIR | |
| 2546 | CCtctgctacttcccaGC | CD80\|VSIR | |
| 2547 | CCtctgctacttcccAGC | CD80\|VSIR | |
| 2548 | CCTctgctacttcccaGC | CD80\|VSIR | |
| 2549 | TCccacgccaaaGC | NT5E\|PDCD1 | |
| 2550 | TCccacgccaaAGC | NT5E\|PDCD1 | |
| 2551 | TCCcacgccaaaGC | NT5E\|PDCD1 | |
| 2552 | TTgaccccaggtCC | HMOX1\|VSIR | |
| 2553 | TTGaccccaggtCC | HMOX1\|VSIR | |
| 2554 | TTgaccccaggTCC | HMOX1\|VSIR | |
| 2555 | CActaccattctCC | CD276\|VSIR | |
| 2556 | CACtaccattcTCC | CD276\|VSIR | |
| 2557 | CACtaccattCTCC | CD276\|VSIR | |
| 2558 | AAaaacatttaCTCC | CD276\|TDO2 | |
| 2559 | AAAaacatttaCTCC | CD276\|TDO2 | |
| 2560 | AAAAacatttaCTCC | CD276\|TDO2 | CRM0286 |
| 2561 | GAGtaagagacTCC | CD80\|KIR2DL1 | |
| 2562 | GAGtaagagaCTCC | CD80\|KIR2DL1 | |
| 2563 | GAGTaagagaCTCC | CD80\|KIR2DL1 | |
| 2564 | ATCCaattttatCC | NT5E\|CD86 | |
| 2565 | ATCCaattttaTCC | NT5E\|CD86 | |
| 2566 | ATCCaattttATCC | NT5E\|CD86 | |
| 2567 | CTtccccagggatCC | CEACAM1\|LAG3 | |
| 2568 | CTTccccagggatCC | CEACAM1\|LAG3 | |
| 2569 | CTtccccagggaTCC | CEACAM1\|LAG3 | |
| 2570 | CAggaaaagactGCC | NT5E\|CD276 | |
| 2571 | CAggaaaagacTGCC | NT5E\|CD276 | |
| 2572 | CAGgaaaagacTGCC | NT5E\|CD276 | |
| 2573 | CCcattttcatgCC | CD276\|VSIR | |
| 2574 | CCcattttcatGCC | CD276\|VSIR | |
| 2575 | CCCattttcatgCC | CD276\|VSIR | |
| 2576 | GCccattttcatgCC | CD276\|VSIR | |
| 2577 | GCccattttcatGCC | CD276\|VSIR | |
| 2578 | GCCcattttcatgCC | CD276\|VSIR | |
| 2579 | CCtctgctacttcCC | CD80\|VSIR | |
| 2580 | CCTctgctacttcCC | CD80\|VSIR | |
| 2581 | CCtctgctacttCCC | CD80\|VSIR | |
| 2582 | TCcctccgagtcCC | CD276\|VSIR | |
| 2583 | TCCctccgagtcCC | CD276\|VSIR | |
| 2584 | TCcctccgagtCCC | CD276\|VSIR | |
| 2585 | GAccccagctcctcCC | TNFRSF14\|VSIR | |
| 2586 | GACcccagctcctcCC | TNFRSF14\|VSIR | |
| 2587 | GAccccagctcctCCC | TNFRSF14\|VSIR | |
| 2588 | GGAaaagactgcCC | NT5E\|CD276 | |
| 2589 | GGAaaagactgCCC | NT5E\|CD276 | |
| 2590 | GGAaaagactGCCC | NT5E\|CD276 | |
| 2591 | AGgaaaagactgcCC | NT5E\|CD276 | |
| 2592 | AGgaaaagactgCCC | NT5E\|CD276 | |
| 2593 | AGgaaaagactGCCC | NT5E\|CD276 | |
| 2594 | CAggaaaagactgcCC | NT5E\|CD276 | |
| 2595 | CAGgaaaagactgcCC | NT5E\|CD276 | |
| 2596 | CAGGaaaagactgcCC | NT5E\|CD276 | |
| 2597 | GTggtcctgggccCC | NT5E\|CD276 | |
| 2598 | GTGgtcctgggccCC | NT5E\|CD276 | |
| 2599 | GTggtcctgggcCCC | NT5E\|CD276 | |
| 2600 | AGTtatgtaaccCC | VTCN1\|VSIR | |
| 2601 | AGTtatgtaacCCC | VTCN1\|VSIR | |
| 2602 | AGTtatgtaaCCCC | VTCN1\|VSIR | |
| 2603 | CAgttatgtaaccCC | VTCN1\|VSIR | |
| 2604 | CAgttatgtaacCCC | VTCN1\|VSIR | |
| 2605 | CAgttatgtaaCCCC | VTCN1\|VSIR | |
| 2606 | CAgttatgtaaCCC | VTCN1\|VSIR | |
| 2607 | CAGttatgtaACCC | VTCN1\|VSIR | |
| 2608 | CAGTtatgtaACCC | VTCN1\|VSIR | |
| 2609 | TAAAaagaggaaCCC | CEACAM1\|CD80 | |
| 2610 | TAAaaagaggaACCC | CEACAM1\|CD80 | |
| 2611 | TAAAaagaggaACCC | CEACAM1\|CD80 | |
| 2612 | CTGAtattcttACC | CEACAM1\|CD274 | |
| 2613 | CTGatattctTACC | CEACAM1\|CD274 | |
| 2614 | CTGAtattctTACC | CEACAM1\|CD274 | |
| 2615 | CCtgggtgtgcacc | HMOX1\|NT5E | |
| 2616 | CCtgggtgtgcACC | HMOX1\|NT5E | |
| 2617 | cCTgggtgtgcacc | HMOX1\|NT5E | |
| 2618 | TAGgctgtgaaaCC | TIGIT\|TDO2 | |
| 2619 | TAGgctgtgaAACC | TIGIT\|TDO2 | |
| 2620 | TAGGctgtgaAACC | TIGIT\|TDO2 | |
| 2621 | TAAttttccttGTAC | NT5E\|CD80 | |
| 2622 | TAATtttccttgTAC | NT5E\|CD80 | |
| 2623 | TAATtttccttGTAC | NT5E\|CD80 | |
| 2624 | CTAattttccttgTAC | NT5E\|CD80 | |
| 2625 | CTAAttttccttgTAC | NT5E\|CD80 | |
| 2626 | CTAAttttccttGTAC | NT5E\|CD80 | |
| 2627 | TCCAtaacttcTAC | CD86\|TDO2 | |
| 2628 | TCCataacttCTAC | CD86\|TDO2 | |
| 2629 | TCCAtaacttCTAC | CD86\|TDO2 | |
| 2630 | TATttttctgccTAC | CD86\|TDO2 | |
| 2631 | TATttttctgcCTAC | CD86\|TDO2 | |
| 2632 | TATTtttctgcCTAC | CD86\|TDO2 | |
| 2633 | TTATttttctgcctAC | CD86\|TDO2 | |
| 2634 | TTATttttctgccTAC | CD86\|TDO2 | |
| 2635 | TTATttttctgcCTAC | CD86\|TDO2 | |
| 2636 | GTGtttgttttATAC | LAG3\|PDCD1LG2 | |
| 2637 | GTGTttgttttaTAC | LAG3\|PDCD1LG2 | |
| 2638 | GTGTttgttttATAC | LAG3\|PDCD1LG2 | |
| 2639 | AGccatagccaTAC | VTCN1\|TIGIT | |
| 2640 | AGCcatagccaTAC | VTCN1\|TIGIT | |
| 2641 | AGCCatagccaTAC | VTCN1\|TIGIT | |
| 2642 | TTcctggtaggGAC | CD276\|KIR2DL1 | |
| 2643 | TTCCtggtagggAC | CD276\|KIR2DL1 | |
| 2644 | TTCCtggtaggGAC | CD276\|KIR2DL1 | |
| 2645 | GTtcctggtagggAC | CD276\|KIR2DL1 | |
| 2646 | GTtcctggtaggGAC | CD276\|KIR2DL1 | |
| 2647 | GTTCctggtaggGAC | CD276\|KIR2DL1 | |
| 2648 | ATGcctctgaggAC | NT5E\|PDCD1LG2 | |
| 2649 | ATGcctctgagGAC | NT5E\|PDCD1LG2 | |
| 2650 | ATGCctctgagGAC | NT5E\|PDCD1LG2 | |
| 2651 | GCtgctaccaggAC | CD80\|TIGIT | |
| 2652 | GCtgctaccagGAC | CD80\|TIGIT | |
| 2653 | GCtgctaccaGGAC | CD80\|TIGIT | |
| 2654 | GGctgctaccaggAC | CD80\|TIGIT | |
| 2655 | GGctgctaccagGAC | CD80\|TIGIT | |
| 2656 | GGCtgctaccaggAC | CD80\|TIGIT | |
| 2657 | AGgctgctaccaggAC | CD80\|TIGIT | |
| 2658 | AGGctgctaccaggAC | CD80\|TIGIT | |
| 2659 | AGgctgctaccagGAC | CD80\|TIGIT | |
| 2660 | TCctacaggtAGAC | CEACAM1\|VTCN1 | |
| 2661 | TCctacaggtAGAC | CEACAMI\|VTCN1 | |
| 2662 | TCCTacaggtAGAC | CEACAM1\|VTCN1 | |
| 2663 | ATcctacaggtAGAC | CEACAM1\|VTCN1 | |
| 2664 | ATCctacaggtAGAC | CEACAM1\|VTCN1 | |
| 2665 | ATCCtacaggtAGAC | CEACAM1\|VTCN1 | |
| 2666 | GCTGcaaagtagAC | CEACAM1\|LAG3 | |
| 2667 | GCTgcaaagtAGAC | CEACAM1\|LAG3 | |
| 2668 | GCTGcaaagtAGAC | CEACAM1\|LAG3 | |
| 2669 | TTACaaccataGAC | CD86\|TDO2 | |
| 2670 | TTAcaaccatAGAC | CD86\|TDO2 | |
| 2671 | TTACaaccatAGAC | CD86\|TDO2 | |
| 2672 | TGttgcaacagAGAC | CD80\|TIGIT | |
| 2673 | TGTTgcaacagaGAC | CD80\|TIGIT | |
| 2674 | TGTTgcaacagAGAC | CD80\|TIGIT | |
| 2675 | TTGTtgcaacagagAC | CD80\|TIGIT | |
| 2676 | TTGttgcaacagAGAC | CD80\|TIGIT | |
| 2677 | TTGTtgcaacagAGAC | CD80\|TIGIT | |
| 2678 | TTCtggttctatCAC | NT5E\|TDO2 | |
| 2679 | TTCtggttctaTCAC | NT5E\|TDO2 | |
| 2680 | TTCTggttctaTCAC | NT5E\|TDO2 | |
| 2681 | AAattcatggGCAC | PDCD1LG2\|TDO2 | |
| 2682 | AAAttcatggGCAC | PDCD1LG2\|TDO2 | |
| 2683 | AAATtcatggGCAC | PDCD1LG2\|TDO2 | |
| 2684 | AGcaggccgcccAC | LAG3\|PDCD1 | |
| 2685 | AGcaggccgccCAC | LAG3\|PDCD1 | |
| 2686 | AGCaggccgcccAC | LAG3\|PDCD1 | |
| 2687 | CAgcaggccgcccAC | LAG3\|PDCD1 | |
| 2688 | CAGcaggccgcccAC | LAG3\|PDCD1 | |
| 2689 | CAgcaggccgccCAC | LAG3\|PDCD1 | |
| 2690 | CCagcaggccgcccAC | LAG3\|PDCD1 | |
| 2691 | CCAgcaggccgcccAC | LAG3\|PDCD1 | |
| 2692 | CCagcaggccgccCAC | LAG3\|PDCD1 | |
| 2693 | TTAtgtaacccCAC | VTCN1\|VSIR | |
| 2694 | TTATgtaacccCAC | VTCN1\|VSIR | |
| 2695 | TTATgtaaccCCAC | VTCN1\|VSIR | |
| 2696 | GTTatgtaaccccAC | VTCN1\|VSIR | |
| 2697 | GTTatgtaacccCAC | VTCN1\|VSIR | |
| 2698 | GTTatgtaaccCCAC | VTCN1\|VSIR | |
| 2699 | AGttatgtaaccccAC | VTCN1\|VSIR | |
| 2700 | AGTTatgtaaccccAC | VTCN1\|VSIR | |
| 2701 | AGTTatgtaacccCAC | VTCN1\|VSIR | |
| 2702 | CAgttatgtaaccccAC | VTCN1\|VSIR | |
| 2703 | CAGttatgtaaccccAC | VTCN1\|VSIR | |
| 2704 | CAGttatgtaacccCAC | VTCN1\|VSIR | |
| 2705 | AACaataccagACAC | NT5E\|CD274 | |
| 2706 | AACAataccagaCAC | NT5E\|CD274 | |
| 2707 | AACAataccagACAC | NT5E\|CD274 | |
| 2708 | TGAAcagacagaCAC | NT5E\|VSIR | |
| 2709 | TGAacagacagACAC | NT5E\|VSIR | |
| 2710 | TGAAcagacagACAC | NT5E\|VSIR | |
| 2711 | AGATaggetgtAAC | NT5E\|CD276 | |
| 2712 | AGAtaggctgTAAC | NT5E\|CD276 | |
| 2713 | AGATaggctgTAAC | NT5E\|CD276 | |
| 2714 | GAGAtaggetgtaAC | NT5E\|CD276 | |
| 2715 | GAGataggetgTAAC | NT5E\|CD276 | |
| 2716 | GAGAtaggctgTAAC | NT5E\|CD276 | |
| 2717 | AGCTgaaattagAAC | HAVCR2\|VTCNl | |
| 2718 | AGCtgaaattaGAAC | HAVCR2\|VTCNl | |
| 2719 | AGCTgaaattaGAAC | HAVCR2\|VTCNl | |
| 2720 | GTTTgatgaccAAC | HAVCR2\|PDCD1LG2 | |
| 2721 | GTTtgatgacCAAC | HAVCR2\|PDCD1LG2 | |
| 2722 | GTTTgatgacCAAC | HAVCR2\|PDCD1LG2 | |
| 2723 | CCctggcttgaAAC | VSIR\|LGALS9 | |
| 2724 | CCCtggcttgaAAC | VSIR\|LGALS9 | |
| 2725 | CCCTggcttgAAAC | VSIR\|LGALS9 | |
| 2726 | GTCaccttgattTTA | HAVCR2\|KIR2DL1\|KIR2DL3 | |
| 2727 | GTCaccttgatTTTA | HAVCR2\|KIR2DL1\|KIR2DL3 | |
| 2728 | GTCAccttgatTTTA | HAVCR2\|KIR2DL1\|KIR2DL3 | |
| 2729 | TGTcaccttgatttTA | HAVCR2\|KIR2DL1\|KIR2DL3 | |
| 2730 | TGTCaccttgatttTA | HAVCR2\|KIR2DL1\|KIR2DL3 | |
| 2731 | TGTCaccttgatTTTA | HAVCR2\|KIR2DL1\|KIR2DL3 | |
| 2732 | CTgtcaccttgatttTA | HAVCR2\|KIR2DL1\|KIR2DL3 | |
| 2733 | CTGtcaccttgattTTA | HAVCR2\|KIR2DL1\|KIR2DL3 | |
| 2734 | CTGTcaccttgatTTTA | HAVCR2\|KIR2DL1\|KIR2DL3 | |
| 2735 | GCtgtcaccttgatttTA | HAVCR2\|KIR2DL1\|KIR2DL3 | |
| 2736 | GCtgtcaccttgattTTA | HAVCR2\|KIR2DL1\|KIR2DL3 | |
| 2737 | GCtgtcaccttgatTTTA | HAVCR2\|KIR2DL1\|KIR2DL3 | |
| 2738 | TACattattttgTTTA | NT5E\|VTCNl | |
| 2739 | TACAttattttgtTTA | NT5E\|VTCNl | |
| 2740 | TACAttattttgTTTA | NT5E\|VTCN1 | |
| 2741 | GAGgagaggaTTTA | NT5E\|PDCD1LG2 | |
| 2742 | GAGGagaggatTTA | NT5E\|PDCD1LG2 | |
| 2743 | GAGGagaggaTTTA | NT5E\|PDCD1LG2 | |
| 2744 | AACAcagggaagTTA | VTCN1\|VSIR | |
| 2745 | AACacagggaaGTTA | VTCN1\|VSIR | |
| 2746 | AACAcagggaaGTTA | VTCN1\|VSIR | |
| 2747 | AGAGttgttttctTA | NT5E\|PDCD1LG2 | |
| 2748 | AGAgttgttttCTTA | NT5E\|PDCD1LG2 | |
| 2749 | AGAGttgttttCTTA | NT5E\|PDCD1LG2 | |
| 2750 | CAGagttgttttcTTA | NT5E\|PDCD1LG2 | |
| 2751 | CAGagttgttttCTTA | NT5E\|PDCD1LG2 | |
| 2752 | CAGAgttgttttcTTA | NT5E\|PDCD1LG2 | |
| 2753 | CTAAttttccttGTA | NT5E\|CD80 | |
| 2754 | CTAattttcctTGTA | NT5E\|CD80 | |
| 2755 | CTAAttttcctTGTA | NT5E\|CD80 | |
| 2756 | ACTAtgaatggGTA | NT5E\|CD86 | |
| 2757 | ACTatgaatgGGTA | NT5E\|CD86 | |
| 2758 | ACTAtgaatgGGTA | NT5E\|CD86 | |
| 2759 | GGgagatttctCTA | NT5E\|CD86 | |
| 2760 | GGGagatttcTCTA | NT5E\|CD86 | |
| 2761 | GGGAgatttcTCTA | NT5E\|CD86 | |
| 2762 | AAGcagcttaGATA | VTCN1\|VSIR | |
| 2763 | AAGCagcttagATA | VTCN1\|VSIR | |
| 2764 | AAGCagcttaGATA | VTCN1\|VSIR | |
| 2765 | GGACagatgaagATA | VTCN1\|TIGIT | |
| 2766 | GGAcagatgaaGATA | VTCN1\|TIGIT | |
| 2767 | GGACagatgaaGATA | VTCN1\|TIGIT | |
| 2768 | ACCcacttagAATA | TIGIT\|TDO2 | |
| 2769 | ACCCacttagaATA | TIGIT\|TDO2 | |
| 2770 | ACCCacttagAATA | TIGIT\|TDO2 | |
| 2771 | CTTGtgtttggtGA | CEACAM1\|CD86 | |
| 2772 | CTTGtgtttggTGA | CEACAM1\|CD86 | |
| 2773 | CTTGtgtttgGTGA | CEACAM1\|CD86 | |
| 2774 | TCTgtagctggtGA | VSIR\|PDCD1LG2 | |
| 2775 | TCtgtagctgGTGA | VSIR\|PDCD1LG2 | |
| 2776 | TCTGtagctgGTGA | VSIR\|PDCD1LG2 | |
| 2777 | TCtatttttaattttcTGA | CD80\|CD86 | |
| 2778 | TCtatttttaattttCTGA | CD80\|CD86 | |
| 2779 | TCTAtttttaattttCTGA | CD80\|CD86 | |
| 2780 | TAgggtcaatCTGA | NT5E\|VTCN1 | |
| 2781 | TAGggtcaatCTGA | NT5E\|VTCN1 | |
| 2782 | TAGGgtcaatCTGA | NT5E\|VTCN1 | |
| 2783 | TGtgtgtgggtgtgGA | CD86\|VSIR | |
| 2784 | TGtgtgtgggtgtGGA | CD86\|VSIR | |
| 2785 | TGTgtgtgggtgtgGA | CD86\|VSIR | |
| 2786 | GTgtgtgtgggtgtgGA | CD86\|VSIR | |
| 2787 | GTGtgtgtgggtgtgGA | CD86\|VSIR | |
| 2788 | GTgtgtgtgggtgtGGA | CD86\|VSIR | |
| 2789 | GGtgtgtgtgggtgtgGA | CD86\|VSIR | |
| 2790 | GGtgtgtgtgggtgtGGA | CD86\|VSIR | |
| 2791 | GGTgtgtgtgggtgtgGA | CD86\|VSIR | |
| 2792 | GTtcctggtaggGA | CD276\|KIR2DL1 | |
| 2793 | GTtcctggtagGGA | CD276\|KIR2DL1 | |
| 2794 | GTTCctggtagGGA | CD276\|KIR2DL1 | |
| 2795 | AGggcagggtcagGA | CD276\|VSIR | |
| 2796 | AGggcagggtcaGGA | CD276\|VSIR | |
| 2797 | AGGgcagggtcagGA | CD276\|VSIR | |
| 2798 | GGctgctaccagGA | CD80\|TIGIT | |
| 2799 | GGctgctaccaGGA | CD80\|TIGIT | |
| 2800 | GGCtgctaccagGA | CD80\|TIGIT | |
| 2801 | AGgctgctaccagGA | CD80\|TIGIT | |
| 2802 | AGGctgctaccagGA | CD80\|TIGIT | |
| 2803 | AGgctgctaccaGGA | CD80\|TIGIT | |
| 2804 | TAgaagagaccaGGA | HAVCR2\|TDO2 | |
| 2805 | TAGaagagaccAGGA | HAVCR2\|TDO2 | |
| 2806 | TAGAagagaccAGGA | HAVCR2\|TDO2 | |
| 2807 | TGgaggtgatTAGA | NT5E\|VTCN1 | |
| 2808 | TGGAggtgattAGA | NT5E\|VTCN1 | |
| 2809 | TGGAggtgatTAGA | NT5E\|VTCN1 | |
| 2810 | ATCctacaggtAGA | CEACAM1\|VTCN1 | |
| 2811 | ATCctacaggTAGA | CEACAM1\|VTCN1 | |
| 2812 | ATCCtacaggTAGA | CEACAM1\|VTCN1 | |
| 2813 | CAtccaacttGAGA | NT5E\|CD80 | |
| 2814 | CATCcaacttgAGA | NT5E\|CD80 | |
| 2815 | CATCcaacttGAGA | NT5E\|CD80 | |
| 2816 | GGactgaagtGAGA | VTCN1\|TIGIT | |
| 2817 | GGActgaagtGAGA | VTCN1\|TIGIT | |
| 2818 | GGACtgaagtGAGA | VTCN1\|TIGIT | |
| 2819 | GCGactgatggaGA | KIR2DL1\|PDCD1LG2 | |
| 2820 | GCGActgatggaGA | KIR2DL1\|PDCD1LG2 | |
| 2821 | GCGactgatgGAGA | KIR2DL1\|PDCD1LG2 | |
| 2822 | GGgccagacaggaGA | CD276\|VSIR | |
| 2823 | GGGccagacaggaGA | CD276\|VSIR | |
| 2824 | GGgccagacaggAGA | CD276\|VSIR | |
| 2825 | AGggccagacaggaGA | CD276\|VSIR | |
| 2826 | AGggccagacaggAGA | CD276\|VSIR | |
| 2827 | AGGgccagacaggaGA | CD276\|VSIR | |
| 2828 | CTAAagaatagaAGA | VTCN1\|TDO2 | |
| 2829 | CTAaagaatagAAGA | VTCN1\|TDO2 | |
| 2830 | CTAAagaatagAAGA | VTCN1\|TDO2 | |
| 2831 | TAGtttctgaaaAAGA | HAVCR2\|IDO1 | |
| 2832 | TAGTttctgaaaaAGA | HAVCR2\|IDO1 | |
| 2833 | TAGTttctgaaaAAGA | HAVCR2\|IDO1 | |
| 2834 | TGtctgtgtgcttCA | NT5E\|LGALS9 | |
| 2835 | TGtctgtgtgcTTCA | NT5E\|LGALS9 | |
| 2836 | TGTCtgtgtgctTCA | NT5E\|LGALS9 | |
| 2837 | CCAcaggaatattCA | NT5E\|CD80 | |
| 2838 | CCACaggaatattCA | NT5E\|CD80 | |
| 2839 | CCACaggaataTTCA | NT5E\|CD80 | |
| 2840 | TGgagcaggcattCA | CEACAM1\|CD276 | |
| 2841 | TGgagcaggcaTTCA | CEACAM1\|CD276 | |
| 2842 | TGGAgcaggcattCA | CEACAM1\|CD276 | |
| 2843 | TCtcaccctgagtCA | CD86\|VSIR | |
| 2844 | TCtcaccctgagTCA | CD86\|VSIR | |
| 2845 | TCtcaccctgaGTCA | CD86\|VSIR | |
| 2846 | GAtgaggaaacagactCA | CD274\|VSIR | |
| 2847 | GAtgaggaaacagaCTCA | CD274\|VSIR | |
| 2848 | GATGaggaaacagaCTCA | CD274\|VSIR | |
| 2849 | AGatgaggaaacagactCA | CD274\|VSIR | |
| 2850 | AGatgaggaaacagaCTCA | CD274\|VSIR | |
| 2851 | AGATgaggaaacagacTCA | CD274\|VSIR | |
| 2852 | AAGcaaatgtctGCA | CEACAM1\|PDCD1LG2 | |
| 2853 | AAGcaaatgtcTGCA | CEACAM1\|PDCD1LG2 | |
| 2854 | AAGCaaatgtcTGCA | CEACAM1\|PDCD1LG2 | |
| 2855 | GGctggtgttggCA | VSIR\|PDCD1LG2 | |
| 2856 | GGctggtgttgGCA | VSIR\|PDCD1LG2 | |
| 2857 | GGCtggtgttggCA | VSIR\|PDCD1LG2 | |
| 2858 | TGgctggtgttggCA | VSIR\|PDCD1LG2 | |
| 2859 | TGGctggtgttggCA | VSIR\|PDCD1LG2 | |
| 2860 | TGgctggtgttgGCA | VSIR\|PDCD1LG2 | |
| 2861 | GTggctggtgttggCA | VSIR\|PDCD1LG2 | |
| 2862 | GTGgctggtgttggCA | VSIR\|PDCD1LG2 | |
| 2863 | GTggctggtgttgGCA | VSIR\|PDCD1LG2 | |
| 2864 | TAGgataatgcagCA | CEACAM1\|CD276 | |
| 2865 | TAGgataatgcaGCA | CEACAM1\|CD276 | |
| 2866 | TAGGataatgcAGCA | CEACAM1\|CD276 | |
| 2867 | ATAggataatgcagCA | CEACAM1\|CD276 | |
| 2868 | ATAggataatgcaGCA | CEACAM1\|CD276 | |
| 2869 | ATAGgataatgcAGCA | CEACAM1\|CD276 | |
| 2870 | AAtgtgggcccagCA | KIR2DL1\|LGALS9 | |
| 2871 | AATgtgggcccagCA | KIR2DL1\|LGALS9 | |
| 2872 | AAtgtgggcccaGCA | KIR2DL1\|LGALS9 | |
| 2873 | CTgaggctcagtcCA | NT5E\|VSIR | |
| 2874 | CTGaggctcagtcCA | NT5E\|VSIR | |
| 2875 | CTgaggctcagtCCA | NT5E\|VSIR | |
| 2876 | CCcattttcatgcCA | CD276\|VSIR | |
| 2877 | CCCattttcatgcCA | CD276\|VSIR | |
| 2878 | CCcattttcatgCCA | CD276\|VSIR | |
| 2879 | GCccattttcatgcCA | CD276\|VSIR | |
| 2880 | GCCcattttcatgcCA | CD276\|VSIR | |
| 2881 | GCccattttcatgCCA | CD276\|VSIR | |
| 2882 | CTctgctacttccCA | CD80\|VSIR | |
| 2883 | CTCtgctacttccCA | CD80\|VSIR | |
| 2884 | CTctgctacttcCCA | CD80\|VSIR | |
| 2885 | CCtctgctacttccCA | CD80\|VSIR | |
| 2886 | CCTctgctacttccCA | CD80\|VSIR | |
| 2887 | CCtctgctacttcCCA | CD80\|VSIR | |
| 2888 | CAgcaggccgccCA | LAG3\|PDCD1 | |
| 2889 | CAGcaggccgccCA | LAG3\|PDCD1 | |
| 2890 | CAgcaggccgcCCA | LAG3\|PDCD1 | |
| 2891 | CCagcaggccgccCA | LAG3\|PDCD1 | |
| 2892 | CCAgcaggccgccCA | LAG3\|PDCD1 | |
| 2893 | CCagcaggccgcCCA | LAG3\|PDCD1 | |
| 2894 | GTTatgtaacccCA | VTCN1\|VSIR | |
| 2895 | GTTatgtaaccCCA | VTCN1\|VSIR | |
| 2896 | GTTatgtaacCCCA | VTCN1\|VSIR | |
| 2897 | AGttatgtaacccCA | VTCN1\|VSIR | |
| 2898 | AGTTatgtaacccCA | VTCN1\|VSIR | |
| 2899 | AGTTatgtaaccCCA | VTCN1\|VSIR | |
| 2900 | CAgttatgtaacccCA | VTCN1\|VSIR | |
| 2901 | CAGttatgtaacccCA | VTCN1\|VSIR | |
| 2902 | CAGTtatgtaacccCA | VTCN1\|VSIR | |
| 2903 | TAttcccaccaccCA | VTCN1\|TDO2 | CRMO287 |
| 2904 | TATtcccaccaccCA | VTCN1\|TDO2 | |
| 2905 | TATTcccaccaccCA | VTCN1\|TDO2 | |
| 2906 | AAaaagaggaaCCCA | CEACAM1\|CD80 | |
| 2907 | AAAaagaggaaCCCA | CEACAM1\|CD80 | |
| 2908 | AAAAagaggaaCCCA | CEACAM1\|CD80 | |
| 2909 | TAaaaagaggaaCCCA | CEACAM1\|CD80 | |
| 2910 | TAAaaagaggaaCCCA | CEACAM1\|CD80 | |
| 2911 | TAAAaagaggaaCCCA | CEACAM1\|CD80 | |
| 2912 | AAttttccttgTACA | NT5E\|CD80 | |
| 2913 | AATtttccttgTACA | NT5E\|CD80 | |
| 2914 | AATTttccttgTACA | NT5E\|CD80 | |
| 2915 | TAattttccttgTACA | NT5E\|CD80 | |
| 2916 | TAAttttccttgTACA | NT5E\|CD80 | |
| 2917 | TAATtttccttgTACA | NT5E\|CD80 | |
| 2918 | CTaattttccttgtACA | NT5E\|CD80 | |
| 2919 | CTaattttccttgTACA | NT5E\|CD80 | |
| 2920 | CTAAttttccttgTACA | NT5E\|CD80 | |
| 2921 | ATTtttctgcctACA | CD86\|TDO2 | |
| 2922 | ATttttctgccTACA | CD86\|TDO2 | |
| 2923 | ATTTttctgccTACA | CD86\|TDO2 | |
| 2924 | TAtttttctgcctACA | CD86\|TDO2 | |
| 2925 | TAtttttctgccTACA | CD86\|TDO2 | |
| 2926 | TATTtttctgccTACA | CD86\|TDO2 | |
| 2927 | TTatttttctgcctaCA | CD86\|TDO2 | |
| 2928 | TTATttttctgcctaCA | CD86\|TDO2 | |
| 2929 | TTATttttctgccTACA | CD86\|TDO2 | |
| 2930 | GCcatagccataCA | VTCN1\|TIGIT | |
| 2931 | GCCatagccataCA | VTCN1\|TIGIT | |
| 2932 | GCCAtagccatACA | VTCN1\|TIGIT | |
| 2933 | AGccatagccataCA | VTCN1\|TIGIT | |
| 2934 | AGccatagccatACA | VTCN1\|TIGIT | |
| 2935 | AGCcatagccatACA | VTCN1\|TIGIT | |
| 2936 | TGAAcagacagacaCA | NT5E\|VSIR | |
| 2937 | TGAacagacagaCACA | NT5E\|VSIR | |
| 2938 | TGAAcagacagaCACA | NT5E\|VSIR | |
| 2939 | GAGataggctGTAA | NT5E\|CD276 | |
| 2940 | GAGAtaggetgTAA | NT5E\|CD276 | |
| 2941 | GAGAtaggctGTAA | NT5E\|CD276 | |
| 2942 | GGagagaggtgagGAA | PDCD1\|KIR2DL1\|KIR2DL3 | |
| 2943 | GGagagaggtgaGGAA | PDCD1\|KIR2DL1\|KIR2DL3 | |
| 2944 | GGAgagaggtgaGGAA | PDCD1\|KIR2DL1\|KIR2DL3 | |
| 2945 | ATTtttaattttctgagGAA | CD80\|CD86 | |
| 2946 | ATttttaattttctgaGGAA | CD80\|CD86 | |
| 2947 | ATTTttaattttctgaGGAA | CD80\|CD86 | |
| 2948 | GGACtgaagtgaGAA | VTCN1\|TIGIT | |
| 2949 | GGActgaagtgAGAA | VTCN1\|TIGIT | |
| 2950 | GGActgaagtgAGAA | VTCN1\|TIGIT | |
| 2951 | CTAAagaatagaaGAA | VTCN1\|TDO2 | |
| 2952 | CTAaagaatagaAGAA | VTCN1\|TDO2 | |
| 2953 | CTAAagaatagaAGAA | VTCN1\|TDO2 | |
| 2954 | TGgcacccttgcAA | VSIR\|PDCD1LG2 | |
| 2955 | TGgcacccttgCAA | VSIR\|PDCD1LG2 | |
| 2956 | TGgcacccttGCAA | VSIR\|PDCD1LG2 | |
| 2957 | AGGataatgcagCAA | CEACAM1\|CD276 | |
| 2958 | AGGataatgcaGCAA | CEACAM1\|CD276 | |
| 2959 | AGGAtaatgcaGCAA | CEACAM1\|CD276 | |
| 2960 | TAGGataatgcagcAA | CEACAM1\|CD276 | |
| 2961 | TAGGataatgcagCAA | CEACAM1\|CD276 | |
| 2962 | TAGGataatgcaGCAA | CEACAM1\|CD276 | |
| 2963 | ATAGgataatgcagcAA | CEACAM1\|CD276 | |
| 2964 | ATAGgataatgcagcAA | CEACAM1\|CD276 | |
| 2965 | ATAGgataatgcaGCAA | CEACAM1\|CD276 | |
| 2966 | GAGgctcagtccAA | NT5E\|VSIR | |
| 2967 | GAGgctcagtcCAA | NT5E\|VSIR | |
| 2968 | GAGgctcagtCCAA | NT5E\|VSIR | |
| 2969 | TGaggctcagtcCAA | NT5E\|VSIR | |
| 2970 | TGAGgctcagtccAA | NT5E\|VSIR | |
| 2971 | TGAGgctcagtcCAA | NT5E\|VSIR | |
| 2972 | CTgaggctcagtccAA | NT5E\|VSIR | |
| 2973 | CTgaggctcagtcCAA | NT5E\|VSIR | |
| 2974 | CTGAggctcagtccAA | NT5E\|VSIR | |
| 2975 | AAaagaggaacCCAA | CEACAM1\|CD80 | |
| 2976 | AAAagaggaacCCAA | CEACAM1\|CD80 | |
| 2977 | AAAAgaggaacCCAA | CEACAM1\|CD80 | |
| 2978 | AAaaagaggaacCCAA | CEACAM1\|CD80 | |
| 2979 | AAAaagaggaacCCAA | CEACAM1\|CD80 | |
| 2980 | AAAAagaggaacCCAA | CEACAM1\|CD80 | |
| 2981 | TAaaaagaggaacCCAA | CEACAM1\|CD80 | |
| 2982 | TAAaaagaggaacCCAA | CEACAM1\|CD80 | |
| 2983 | TAAAaagaggaacCCAA | CEACAM1\|CD80 | |
| 2984 | ATTttccttgtACAA | NT5E\|CD80 | |
| 2985 | ATTTtccttgtaCAA | NT5E\|CD80 | |
| 2986 | ATTTtccttgtACAA | NT5E\|CD80 | |
| 2987 | AATtttccttgtACAA | NT5E\|CD80 | |
| 2988 | AATTttccttgtaCAA | NT5E\|CD80 | |
| 2989 | AATTttccttgtACAA | NT5E\|CD80 | |
| 2990 | TAAttttccttgtACAA | NT5E\|CD80 | |
| 2991 | TAATtttccttgtaCAA | NT5E\|CD80 | |
| 2992 | TAATtttccttgtACAA | NT5E\|CD80 | |
| 2993 | CTAAttttccttgtacAA | NT5E\|CD80 | |
| 2994 | CTAattttccttgtaCAA | NT5E\|CD80 | |
| 2995 | CTAAttttccttgtACAA | NT5E\|CD80 | |
| 2996 | TAaagaatagaaGAAA | VTCN1\|TDO2 | |
| 2997 | TAAagaatagaaGAAA | VTCN1\|TDO2 | |
| 2998 | TAAAgaatagaaGAAA | VTCN1\|TDO2 | |
| 2999 | CTaaagaatagaaGAAA | VTCN1\|TDO2 | |
| 3000 | CTAaagaatagaaGAAA | VTCN1\|TDO2 | |
| 3001 | CTAAagaatagaaGAAA | VTCN1\|TDO2 | |
| 3002 | GAggctcagtccaAA | NT5E\|VSIR | |
| 3003 | GAggctcagtcCAAA | NT5E\|VSIR | |
| 3004 | GAGGctcagtcCAAA | NT5E\|VSIR | |
| 3005 | TGaggctcagtccaAA | NT5E\|VSIR | |
| 3006 | TGAggctcagtccaAA | NT5E\|VSIR | |
| 3007 | TGAggctcagtcCAAA | NT5E\|VSIR | |
| 3008 | CTgaggctcagtccaAA | NT5E\|VSIR | |
| 3009 | CTGaggctcagtccAAA | NT5E\|VSIR | |
| 3010 | CTGAggctcagtccaAA | NT5E\|VSIR | |
| 3011 | AAAGaggaacccAAA | CEACAM1\|CD80 | |
| 3012 | AAAgaggaaccCAAA | CEACAM1\|CD80 | |
| 3013 | AAAGaggaaccCAAA | CEACAM1\|CD80 | |
| 3014 | AAaagaggaaccCAAA | CEACAM1\|CD80 | |
| 3015 | AAAagaggaaccCAAA | CEACAM1\|CD80 | |
| 3016 | AAAAgaggaaccCAAA | CEACAM1\|CD80 | |
| 3017 | AAaaagaggaaccCAAA | CEACAM1\|CD80 | |
| 3018 | AAAaagaggaaccCAAA | CEACAM1\|CD80 | |
| 3019 | AAAAagaggaaccCAAA | CEACAM1\|CD80 | |
| 3020 | TAaaaagaggaaccCAAA | CEACAM1\|CD80 | |
| 3021 | TAAaaagaggaaccCAAA | CEACAM1\|CD80 | |
| 3022 | TAAAaagaggaaccCAAA | CEACAM1\|CD80 | |
| 3023 | TAAagaatagaagAAAA | VTCN1\|TDO2 | |
| 3024 | TAAAgaatagaagaAAA | VTCN1\|TDO2 | |
| 3025 | TAAAgaatagaagAAAA | VTCN1\|TDO2 | |
| 3026 | CTAAagaatagaagaAAA | VTCN1\|TDO2 | |
| 3027 | CTAaagaatagaagAAAA | VTCN1\|TDO2 | |
| 3028 | CTAAagaatagaagAAAA | VTCN1\|TDO2 | |
| 3029 | AAGAggaacccaaAA | CEACAM1\|CD80 | |
| 3030 | AAGAggaacccaAAA | CEACAM1\|CD80 | |
| 3031 | AAGAggaacccAAAA | CEACAM1\|CD80 | |
| 3032 | AAAGaggaacccaaAA | CEACAM1\|CD80 | |
| 3033 | AAAGaggaacccaAAA | CEACAM1\|CD80 | |
| 3034 | AAAGaggaacccAAAA | CEACAM1\|CD80 | |
| 3035 | AAAAgaggaacccaaAA | CEACAM1\|CD80 | |
| 3036 | AAAAgaggaacccaAAA | CEACAM1\|CD80 | |
| 3037 | AAAAgaggaacccAAAA | CEACAM1\|CD80 | |
| 3038 | AAAaagaggaacccAAAA | CEACAM1\|CD80 | |
| 3039 | AAAAagaggaacccaAAA | CEACAM1\|CD80 | |
| 3040 | AAAAagaggaacccAAAA | CEACAM1\|CD80 | |
| 3041 | TAAaaagaggaacccAAAA | CEACAM1\|CD80 | |
| 3042 | TAAAaagaggaacccaAAA | CEACAM1\|CD80 | |
| 3043 | TAAAaagaggaacccAAAA | CEACAM1\|CD80 | |

Example 5. Design of LNA-Modified Antisense Oligonucleotides for Knockdown of Targets in both Human and Mouse.

LNA antisense oligonucleotides that can effectively knock down targets listed in Table 1.1 and 1.2 in both human and mouse were designed. In this example, the target regions are shared by orthologous sequences in human and mouse (Table 4.1: SEQ ID NOs: 1473-1503).

TABLE 4.1

| SEQ ID NO | target sequence (5′-3′) | target |
|---|---|---|
| 1473 | UGAAAGUCAAUGGUAAGAAU | CD274 |
| 1474 | UGAAAGUCAAUGGUAAG | CD274 |
| 1475 | CCUGGCUUUCGUGUGCU | CD276 |
| 1476 | ACAGACACCAAACAGCU | CD276 |
| 1477 | CGUGUGCUGGAGAAAGA | CD276 |
| 1478 | UUUCGUGUGCUGGAGAA | CD276 |
| 1479 | GUGUGCUGGAGAAAGAUCAA | CD276 |
| 1480 | UCAGAAAACAAAAGAUC | CD80 |
| 1481 | UUAGAAUAUUACCUCAU | CD86 |
| 1482 | CGAUUCUGCUUCUAG | CD86 |
| 1483 | CAUAAAUUUGACCUGC | CD86 |
| 1484 | UUGUAUGCAAAUAGGC | CD86 |
| 1485 | UCUCUAGUCAGUUCCC | CD86 |
| 1486 | UUAGCCCUGAAACUGAC | CD86 |
| 1487 | UAGUAUUUUGGCAGGA | CD86 |
| 1488 | UCUUACAACAGGGGUCUAU | CTLA4 |
| 1489 | GGUUUGAAUAUAAACACUAU | CTLA4 |
| 1490 | CAGCCUUAUUUUAUUCCCAU | CTLA4 |
| 1491 | CAGGGGUCUAUGUGAAAAUG | CTLA4 |
| 1492 | CAGAGCCAGAAUGUGAAAAG | CTLA4 |
| 1493 | GCCUUAUUUUAUUCCCAUCA | CTLA4 |
| 1494 | GAGAAUGCUGAGUUCAU | HMOX1 |
| 1495 | GUCUCUCUAUUGGUGGAAAU | IDO1 |
| 1496 | AGAUGUUCUCUGUAAGUCUA | LGALS9 |
| 1497 | AUGCCACCAUUGUCUU | PDCD1 |
| 1498 | GAAAGUCAAAGGUGAGU | PDCD1LG2 |
| 1499 | CGCCUGGGACUACAAGU | PDCD1LG2 |
| 1500 | AUCAAAGUGACAGGUGGGU | VTCN1 |
| 1501 | GAGAAUGUGACCAUGAAGGU | VTCN1 |
| 1502 | GACUGGUUUUGCUGGAGGAU | VTCN1 |
| 1503 | CUGAGAAUGUGACCAUGAAG | VTCN1 |

TABLE 4.2

| SEQ ID NO | target sequence (5′-3′) | target |
|---|---|---|
| 3044 | CCACAGUAAGUAAAGCCA | CEACAM1 |
| 3045 | AACGUAUAUGAAGUGGAG | HAVCR2 |
| 3046 | CACCUACAGAGAUGGCUU | LAG3 |
| 3047 | AUAAUUAUUCUACCCAGG | NT5E |
| 3048 | CACCAAGUGUCGAGUGC | NT5E |
| 3049 | GGGAAGUACCCAUUCAUA | NT5E |
| 3050 | ACCAGCUUCUGGCCAUUU | TIGIT |
| 3051 | AAAGGGCACGAUGUGAC | VSIR |
| 3052 | CAAUAAACACAUCUGAGA | VSIR |

The LNA ASOs listed in Table 5.1 below (Table 5.1: SEQ ID NOs: 1504-1534; LNA shown in uppercase, DNA in lowercase), were designed against each of the target sites listed in Table 4.1 above.

TABLE 5.1

| SEQ ID NO | Oligonucleotide | target |
|---|---|---|
| 1504 | ATtcttaccattgactttCA | CD274 |
| 1505 | CTTAccattgactttCA | CD274 |
| 1506 | AGcacacgaaagccaGG | CD276 |
| 1507 | AGctgtttggtgtctGT | CD276 |
| 1508 | TCTttctccagcacACG | CD276 |
| 1509 | TTCtccagcacacGAAA | CD276 |
| 1510 | TTgatctttctccagcacAC | CD276 |
| 1511 | GATCttttgttttctGA | CD80 |
| 1512 | ATGAggtaatattCTAA | CD86 |
| 1513 | CTAGaagcagaATCG | CD86 |
| 1514 | GCAggtcaaatttATG | CD86 |
| 1515 | GCctatttgcataCAA | CD86 |
| 1516 | GGgaactgactaGAGA | CD86 |
| 1517 | GTcagtttcagggcTAA | CD86 |
| 1518 | TCCTgccaaaataCTA | CD86 |
| 1519 | ATagacccctgttgtaaGA | CTLA4 |
| 1520 | ATAgtgtttatattcaAACC | CTLA4 |
| 1521 | ATGGgaataaaataaggcTG | CTLA4 |
| 1522 | CAttttcacatagaccccTG | CTLA4 |
| 1523 | CTtttcacattctggctcTG | CTLA4 |
| 1524 | TGatgggaataaaataaGGC | CTLA4 |
| 1525 | ATgaactcagcatTCTC | HMOX1 |
| 1526 | ATTtccaccaatagagagAC | IDO1 |
| 1527 | TAGacttacagagaacaTCT | LGALS9 |
| 1528 | AAGacaatggtgGCAT | PDCD1 |
| 1529 | ACTCacctttgacttTC | PDCD1LG2 |
| 1530 | ACttgtagtcccaggCG | PDCD1LG2 |
| 1531 | ACccacctgtcactttgAT | VTCN1 |
| 1532 | ACcttcatggtcacattcTC | VTCN1 |
| 1533 | ATcctccagcaaaaccagTC | VTCN1 |
| 1534 | CTtcatggtcacattctcAG | VTCN1 |

The LNA ASOs listed in Table 5.2 below (Table 5.2: SEQ ID NOs: 3053-3061; LNA shown in uppercase, DNA in lowercase), were designed against each of the target sites listed in Table 4.2 above.

TABLE 5.2

| SEQ ID NO | Oligonucleotide | target |
|---|---|---|
| 3053 | TGgctttacttactgtGG | CEACAM1 |
| 3054 | CTccacttcatatacGTT | HAVCR2 |
| 3055 | AAgccatctctgtaggTG | LAG3 |
| 3056 | CCtgggtagaataaTTAT | NT5E |
| 3057 | GCactcgacacttggTG | NT5E |
| 3058 | TAtgaatgggtacttcCC | NT5E |
| 3059 | AAAtggccagaagctgGT | TIGIT |
| 3060 | GTcacatcgtgccctTT | VSIR |
| 3061 | TCTcagatgtgtttaTTG | VSIR |

Example 6. Design of LNA-Modified Antisense Oligonucleotides for Knockdown of Targets in Human.

LNA antisense oligonucleotides that can effectively knock down targets listed in Table 1.1 and 1.2 in human were designed. In this example, the target regions are listed in Table 6.1 and 6.2 (Table 6.1: SEQ ID NOs: 1535-1593 and 1654 and Table 6.2: SEQ ID NOs: 3062-3097). These target regions are selected so that they will not be identical to target regions in other immune checkpoint proteins, and so that there will be a minimum of off target effects. The target regions in Table 6.1 and 6.2 are therefore preferred target regions. LNA ASOs were designed against each of these target sites (Table 7.1: SEQ ID NOs: 1594-1653 and Table 7.2: SEQ ID NOs: 3098-3133).

TABLE 6.1
Preferred target regions in Immune Checkpoint
Proteins. (These target regions are targeted by
the oligonucleotides described in Table 7.1).

| SEQ ID NO | target sequence (5′-3′) | target | oligoID |
|---|---|---|---|
| 1535 | GCCGUUUUGUAUUAACU | CD274 | |
| 1536 | CGACCAGAUAAAGUGAU | CD274 | |
| 1537 | UUAUCACUAUCACUUCG | CD274 | |
| 1538 | ACGUAUCUUAAUCCUGA | CD274 | |
| 1539 | CGGGGUGAAUAGGUGUU | CD276 | |
| 1540 | CAAAUACGACAGAGGCU | CD276 | |
| 1541 | GUACGAUUCUUCAUCUC | CD276 | |
| 1542 | GCCUCGUCCAUUCCCAC | CD276 | |
| 1543 | GACCACCCACAACCUUA | CD276 | |
| 1544 | GAGCAUAGGUAAUCGUA | CD276 | |
| 1545 | CCCAUCUACGUCCCUCA | CD276 | |
| 1546 | ACCCACUACCUCACCUU | CD80 | |
| 1547 | GAAAACGGAGUGCAAC | CD80 | |
| 1548 | AUUACUACACCCGCCA | CD80 | |
| 1549 | GUGGACGGAGAUUAGU | CD86 | |
| 1550 | CGAAGAUGGAUAGGAAC | CD86 | |
| 1551 | AUGGUAAUAUGUCGUAA | CD86 | |
| 1552 | UGAAGACCUGAACACCG | CTLA4 | |
| 1553 | ACACCGCUCCCAUAAAG | CTLA4 | |
| 1554 | CCCAACGAAAAGCACAU | HMOX1 | |
| 1555 | ACGCCCACCUGUUAAU | HMOX1 | |
| 1556 | CUCGAAUUUGCCUCUGA | HMOX1 | |
| 1557 | GUUGACGGGAUAAUAGA | IDO1 | |
| 1558 | AGGUAGACGGGCGAGU | LGALS9 | |
| 1559 | CGUCGUUCAGUGGGGAU | LGALS9 | |
| 1560 | CUUAAACUAACGCAGG | LGALS9 | |
| 1561 | CGGUGGAUAAAGGUUCA | LGALS9 | |
| 1562 | CUGGUGGUUGGUGUCGU | PDCD1 | |
| 1563 | GUUCGAGUGAGGACAGU | PDCD1 | |
| 1564 | GUCCUGUAAUGCGGUCU | PDCD1 | |
| 1565 | GUCUGGGCGGUGCUACA | PDCD1 | |
| 1566 | CGGAAACGAAGAGUAU | PDCD1LG2 | |
| 1567 | GUCGUUCGUUAUAUGG | PDCD1LG2 | |
| 1568 | AGGUUACUCCACUUCG | PDCD1LG2 | |
| 1569 | CCGCUGUGAGACCAUU | TNFRSF14 | |
| 1570 | CGGUCGGCAAGGUUGU | TNFRSF14 | |
| 1571 | GCGGCAGGUUAUCGUG | TNFRSF14 | |
| 1572 | CGUAGGUCGUCAUAGG | VTCN1 | |
| 1573 | UUACGAGGCAUGAUAG | VTCN1 | |
| 1574 | UGUGUCCCGUAUCGCC | VTCN1 | |
| 1575 | GCGAUGCGACUAUGAC | VTCN1 | |
| 1576 | GAGACUACGAGAGUAA | VTCN1 | |
| 1577 | GUUGCCUGACCUACGU | CTLA4 | CRM0095 |
| 1578 | AUGACGUUUGAUCUGUAC | CTLA4 | CRM0096 |
| 1579 | AAAGUGUACCUGUUCG | PDCD1 | CRM0097 |
| 1580 | UUCGUGCUAAACUGGUAC | PDCD1 | CRM0098 |
| 1581 | AUCACUCUCCAGAUACAC | CD274 | CRMO129 |
| 1582 | AUCACUCUCCAGAUACACA | CD274 | CRM0130 |
| 1583 | AAAGUCAAUGGUAAGAAUUA | CD274 | CRMO131 |
| 1584 | GUGUGGGUUCAAACACAU | CTLA4 | CRMO132 |
| 1585 | UCUGUGUGGGUUCAAACA | CTLA4 | CRMO133 |
| 1586 | CAGUCCGUGAGUUUGUC | IDO1 | CRMO134 |
| 1587 | GUCUCUCUAUUGGUGGA | IDO1 | CRM0135 |
| 1588 | UAUGCCACCAUUGUCUU | PDCD1 | CRMO136 |
| 1589 | CACCUUCACCUGCAGCUU | PDCD1 | CRMO137 |
| 1590 | GUCACCAGUGUUCUGCG | PDCD1LG2 | CRMO138 |
| 1591 | GAAAGUCAAAGGUGAGUG | PDCD1LG2 | CRMO139 |
| 1592 | AGAGGGCAGGACAUUU | CTLA4 | CRM0104 |
| 1593 | AGAGGGCAGGACAUUU | CTLA4 | CRM0105 |
| 1654 | GAGUAUUCAUAGCGGA | IDO1 | CRMO187 |

TABLE 6.2
Preferred target regions in Immune Checkpoint
Proteins. (These target regions are targeted by
the oligonucleotides described in Table 7.2)

| SEQ ID NO | target sequence (5′-3′) | target | oligoID |
|---|---|---|---|
| 3062 | GGGACGUAUUGGUGUG | CEACAM1 | |
| 3063 | CCUGCCUCUAUUACGGA | CEACAM1 | |
| 3064 | GUUUCUGCGAUUAUGGU | HAVCR2 | |
| 3065 | CCCUAAACUAUGCGUG | HAVCR2 | |
| 3066 | GGCUCUUAUCUUCGGC | HAVCR2 | |
| 3067 | ACUCUAUUCCGUGUUAC | HAVCR2 | |
| 3068 | GGUGGUUGUAAUGUAUAA | HAVCR2 | |
| 3069 | CAGGGUUAGACUACGGU | KIR2DL1 | |
| 3070 | GCUCCCUCUUAACGCA | KIR2DL1 | |
| 3071 | AGUUCUAGGAUGACACAA | KIR2DL1 | |
| 3072 | CCGACGUGAUGAAACAUU | KIR2DL3 | |
| 3073 | UUAGCUCUGUAUAUGGGU | KIR2DL3 | |
| 3074 | GUAGCCAUAGAAACGUG | KIR2DL3 | |
| 3075 | CACCAUGUCUUUGCGGG | KIR2DL3 | |
| 3076 | ACCGCAAUUCCAUCUAC | KIR2DL3 | |
| 3077 | AUAAAUCCGUCCCUUGG | LAG3 | |
| 3078 | GCCGCCUUACCCUGAAC | LAG3 | |
| 3079 | GCGACUUUACCCUUCGA | LAG3 | |
| 3080 | CUUGUCCUCGUACUAUU | NT5E | |
| 3081 | ACUUCCGCUUACCGCU | NT5E | |
| 3082 | AGCCCGUCUACUUGUC | NT5E | |
| 3083 | GCUUGACUACUGAUAAC | NT5E | |
| 3084 | ACAUGAUAAGAUACACU | TDO2 | |
| 3085 | UGAUGAGAUUCGUCCAU | TDO2 | |
| 3086 | GCAAUUCCGACUAUUAG | TDO2 | CRM0288 |
| 3087 | CGUCCCUACUGUUAUUA | TDO2 | CRM0289 |
| 3088 | CACAAGUACGAAACAUA | TDO2 | CRM0290 |
| 3089 | GGUAACUGAACCGCUU | TIGIT | |
| 3090 | CCUAUCCUAAUACUAUCU | TIGIT | |
| 3091 | AGACCUUAUGCGAUGCU | TIGIT | |
| 3092 | ACUAACUAUUUACCCUAU | TIGIT | |
| 3093 | UUCGGCUUACUCUAUA | TIGIT | |
| 3094 | CCUAUACUGUUGACCCA | TIGIT | |
| 3095 | CGUGAAUACUGUGUAAU | VSIR | |
| 3096 | CUUAGUGCGGUGUCGG | VSIR | |
| 3097 | UGACGCUUCCUUUCUUAG | VSIR | |

TABLE 7.1
LNA ASOs targeting the target regions listed in
Table 6.1 (LNA shown in uppercase, DNA lowercase)

| SEQ | | | | Target |
|---|---|---|---|---|
| ID | | | | region is |
| NO | Oligonucleotide | target | oligoID | SEQ ID NO |
| 1594 | AGTTaatacaaaaCGGC | CD274 | | 1535 |
| 1595 | ATcactttatctgGTCG | CD274 | | 1536 |
| 1596 | CGAAgtgatagtgATAA | CD274 | | 1537 |
| 1597 | TCAggattaagataCGT | CD274 | CRM0185 | 1538 |
| 1598 | AAcacctattcaccCCG | CD276 | | 1539 |
| 1599 | AGcctctgtcgtattTG | CD276 | | 1540 |
| 1600 | GAGatgaagaatcGTAC | CD276 | | 1541 |
| 1601 | GTgggaatggacgagGC | CD276 | | 1542 |
| 1602 | TAaggttgtgggtggTC | CD276 | | 1543 |
| 1603 | TAcgattacctatgCTC | CD276 | | 1544 |
| 1604 | TGagggacgtagatGGG | CD276 | | 1545 |
| 1605 | AAggtgaggtagtggGT | CD80 | | 1546 |
| 1606 | GTtgcactccgttTTC | CD80 | | 1547 |
| 1607 | TGgcgggtgtagTAAT | CD80 | | 1548 |
| 1608 | ACtaatctccgtcCAC | CD86 | | 1549 |
| 1609 | GTtcctatccatcttCG | CD86 | | 1550 |
| 1610 | TTACgacatattaCCAT | CD86 | | 1551 |
| 1611 | CGgtgttcaggtcttCA | CTLA4 | | 1552 |
| 1612 | CTttatgggagcggTGT | CTLA4 | | 1553 |
| 1613 | ATGtgcttttcgttgGG | HMOX1 | | 1554 |
| 1614 | ATtaacaggtgggCGT | HMOX1 | | 1555 |
| 1615 | TCagaggcaaattCGAG | HMOX1 | | 1556 |
| 1616 | TCTattatcccgtcaAC | IDO1 | | 1557 |
| 1617 | ACtcgcccgtctacCT | LGALS9 | | 1558 |
| 1618 | ATCCccactgaacgaCG | LGALS9 | | 1559 |
| 1619 | CCTgcgttagtttaAG | LGALS9 | | 1560 |
| 1620 | TGAacctttatccacCG | LGALS9 | | 1561 |
| 1621 | ACGacaccaaccaccAG | PDCD1 | | 1562 |
| 1622 | ACTgtcctcactcgaAC | PDCD1 | | 1563 |
| 1623 | AGAccgcattacaggAC | PDCD1 | | 1564 |
| 1624 | TGtagcaccgcccagAC | PDCD1 | | 1565 |
| 1625 | ATActcttcgtttcCG | PDCD1LG2 | CRM0190 | 1566 |
| 1626 | CCAtataacgaaCGAC | PDCD1LG2 | | 1567 |
| 1627 | CGAagtggagtaaCCT | PDCD1LG2 | | 1568 |
| 1628 | AAtggtctcacagCGG | TNFRSF14 | | 1569 |
| 1629 | ACAAccttgccgacCG | TNFRSF14 | | 1570 |
| 1630 | CACgataacctgccGC | TNFRSF14 | | 1571 |
| 1631 | CCtatgacgacctACG | VTCN1 | | 1572 |
| 1632 | CTAtcatgcctcgTAA | VTCN1 | | 1573 |
| 1633 | GGcgatacgggacaCA | VTCN1 | | 1574 |
| 1634 | GTcatagtcgcatcGC | VTCN1 | | 1575 |
| 1635 | TTActctcgtagtCTC | VTCN1 | | 1576 |
| 1636 | ACGTaggtcaggcAAC | CTLA4 | CRM0095 | 1577 |
| 1637 | GTACagatcaaacgtCAT | CTLA4 | CRM0096 | 1578 |
| 1638 | CGAAcaggtacaCTTT | PDCD1 | CRM0097 | 1579 |
| 1639 | GTACcagtttagcacGAA | PDCD1 | CRM0098 | 1580 |
| 1640 | GTgtatctggagagtGAT | CD274 | CRM0129 | 1581 |
| 1641 | TGtgtatctggagagtgAT | CD274 | CRM0130 | 1582 |
| 1642 | TAAttcttaccattgaCTTT | CD274 | CRM0131 | 1583 |
| 1643 | ATgtgtttgaacccacAC | CTLA4 | CRM0132 | 1584 |
| 1644 | TGtttgaacccacacaGA | CTLA4 | CRM0133 | 1585 |
| 1645 | GACAaactcacggacTG | IDO1 | CRM0134 | 1586 |
| 1646 | TCcaccaatagagAGAC | IDO1 | CRM0135 | 1587 |
| 1647 | AAgacaatggtggCATA | PDCD1 | CRM0136 | 1588 |
| 1648 | AAgctgcaggtgaaggTG | PDCD1 | CRM0137 | 1589 |
| 1649 | CGcagaacactggtGAC | PDCD1LG2 | CRM0138 | 1590 |
| 1650 | CActcacctttgacttTC | PDCD1LG2 | CRM0139 | 1591 |
| 1651 | AAAtgtcctgccctCT | CTLA4 | CRM0104 | 1592 |
| 1652 | AAatgTcctgccctCT | CTLA4 | CRM0105 | 1593 |
| 1653 | TCCgctatgaatacTC | IDO1 | CRM0187 | 1654 |

TABLE 7.2
LNA ASOs targeting the target regions listed in
Table 6.2 (LNA shown in uppercase, DNA lowercase)

| SEQ | | | | Target |
|---|---|---|---|---|
| ID | | | | region is |
| NO | Oligonucleotide | target | oligoID | SEQ ID NO |
| 3098 | CAcaccaatacgtcCC | CEACAM1 | | 3062 |
| 3099 | TCcgtaatagaggcaGG | CEACAM1 | | 3063 |
| 3100 | ACCAtaatcgcagaAAC | HAVCR2 | | 3064 |
| 3101 | CACgcatagtttagGG | HAVCR2 | | 3065 |
| 3102 | GCcgaagataagagCC | HAVCR2 | | 3066 |
| 3103 | GTaacacggaataGAGT | HAVCR2 | | 3067 |
| 3104 | TTAtacattacaacCACC | HAVCR2 | | 3068 |
| 3105 | ACcgtagtctaacccTG | KIR2DL1 | | 3069 |
| 3106 | TGcgttaagagggaGC | KIR2DL1 | | 3070 |
| 3107 | TTgtgtcatcctagaaCT | KIR2DL1 | | 3071 |
| 3108 | AATgtttcatcacgtcGG | KIR2DL3 | | 3072 |
| 3109 | ACCcatatacagagcTAA | KIR2DL3 | | 3073 |
| 3110 | CACGtttctatggctAC | KIR2DL3 | | 3074 |
| 3111 | CCCgcaaagacatggTG | KIR2DL3 | | 3075 |
| 3112 | GTagatggaattgcGGT | KIR2DL3 | | 3076 |
| 3113 | CCaagggacggattTAT | LAG3 | | 3077 |
| 3114 | GTtcagggtaaggcgGC | LAG3 | | 3078 |
| 3115 | TCgaagggtaaagtCGC | LAG3 | | 3079 |
| 3116 | AATAgtacgaggaCAAG | NT5E | | 3080 |
| 3117 | AGcggtaagcggaAGT | NT5E | | 3081 |
| 3118 | GACaagtagacgggCT | NT5E | | 3082 |
| 3119 | GTtatcagtagtcaAGC | NT5E | | 3083 |
| 3120 | AGTgtatcttatcaTGT | TD02 | | 3084 |
| 3121 | ATGgacgaatctcatCA | TD02 | | 3085 |
| 3122 | CTaatagtcggaatTGC | TD02 | CRM0288 | 3086 |
| 3123 | TAATaacagtaggGACG | TD02 | CRM0289 | 3087 |
| 3124 | TAtgtttcgtacttGTG | TD02 | CRM0290 | 3088 |
| 3125 | AAgcggttcagttACC | TIGIT | | 3089 |
| 3126 | AGATagtattaggatAGG | TIGIT | | 3090 |
| 3127 | AGcatcgcataaggtCT | TIGIT | | 3091 |
| 3128 | ATagggtaaatagtTAGT | TIGIT | | 3092 |
| 3129 | TATagagtaagcCGAA | TIGIT | | 3093 |
| 3130 | TGggtcaacagtataGG | TIGIT | | 3094 |
| 3131 | ATTAcacagtattcaCG | VSIR | | 3095 |
| 3132 | CCgacaccgcactaAG | VSIR | | 3096 |
| 3133 | CTAAgaaaggaagcgtCA | VSIR | | 3097 |

Example 7. Antisense Oligonucleotide-Mediated Knockdown of Immune Checkpoint Proteins in Cultured Cancer Cells

Chronic myelogenous leukemia cell line K562 (ECACC cat. no. 89121407) was purchased from Sigma and maintained in RPMI1640 medium (Sigma cat. no. R0883) supplemented with 10% fetal calf serum (Sigma cat. no. F2442), 2 mM L-glutamine (Sigma cat. no. G7513) and penicillin/streptomycin (Sigma cat. no. P4333) in a humidified 5% CO2 incubator at 37° C. and passaged twice a week.

For unassisted uptake of the immune checkpoint-targeting antisense oligonucleotides listed in Table 7.1, K562 cells were seeded in 12-well cell culture plates and transfected essentially as described in Soifer et al. (Methods Mol Biol. 2012; 815: 333-46) using ASOs in a concentration range of 0.1 μM-2.5 μM final concentration. A scrambled oligonucleotide and mock transfection were included as controls. Three to six days after transfection total RNA was isolated from the cells using the RNeasy mini kit (Qiagen) according to the manufacturer's instructions and 1 μg total RNA was reverse transcribed into cDNA using the High Capacity cDNA reverse transcription kit (Life Technologies cat. no. 4374967) according to the protocol provided by the manufacturer.

Target mRNA levels were determined by quantitative RT-PCR using Taqman Gene Expression Master Mix (ABI cat. no. 4369542) and pre-designed Taqman assays for CTLA-4 (IDT Hs.PT.58.3907580) and PDCD1 (IDT Hs.PT.58.39641096). Furthermore, the expression of GAPDH mRNA was measured (IDT Hs.PT.58.40035104) and used as an endogenous control. qRT-PCR reactions were carried out on a Quantstudio 6 Flex Real-Time thermocycler (ABI).

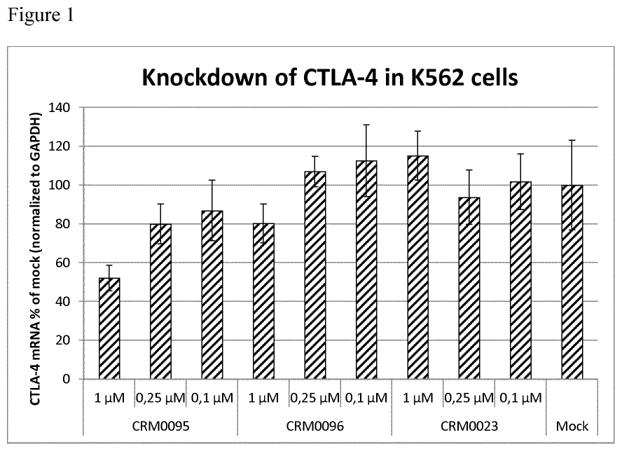

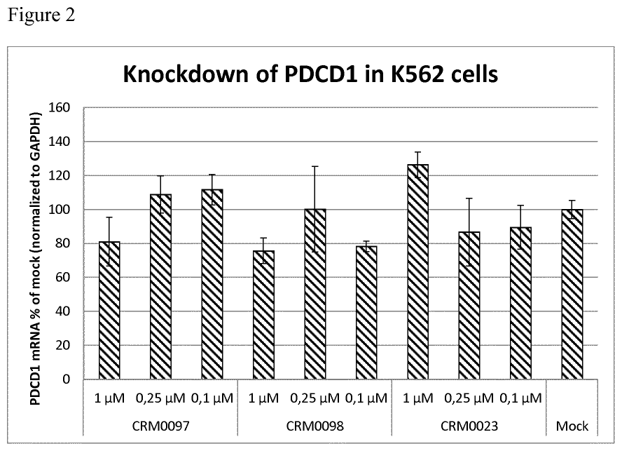

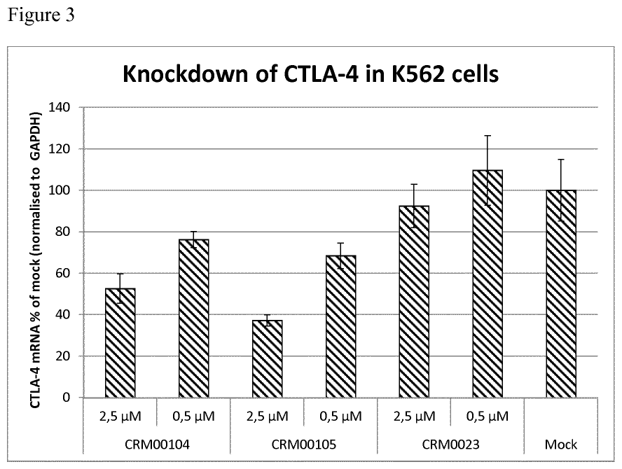

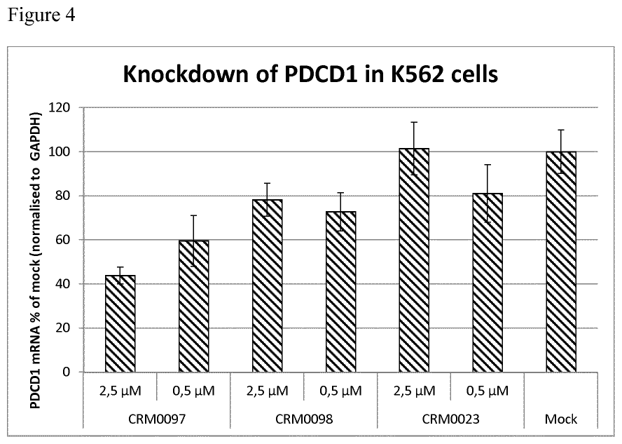

Examples of ASO-mediated CTLA-4 and PDCD1 knockdown in K562 cells using ASO's with oligo id's: CRM0095, CRM0096, CRM0097, CRM0098, CRM0104 and CRM0105 (listed in Table 7.1), are shown in FIGS. 1, 2, 3 and 4.

Example 8. Antisense-Mediated Knockdown of Immune Checkpoint-Encoding mRNAs in Cultured Cancer Cells Using Bispecific Antisense Oligonucleotides

Human glioblastoma cell line GMS-10 (DSMZ cat. no. ACC405) was purchased from Leibniz Institue DSMZ-German Collection of Microorganisms and Cell Cultures and maintained in 85-90% Dulbecco's MEM (Sigma cat. no. D6546), 10-15% fetal bovine serum (Sigma cat. no. F2442), 2 mM L-glutamine (Sigma cat. no. G7513), and penicillin/streptomycin (Sigma cat. no. P4333) in a humidified 5% CO2 incubator at 37° C. and passaged twice a week.

For transfection of the immune checkpoint-targeting antisense oligonucleotides listed in Table 3.1 and 3.2, GMS-10 cells were seeded in 6-well cell culture plates and transfected using 5 μL/mL Lipofectamine 2000 (Thermo Fisher Scientific cat. no. 11668027) using antisense oligonucleotides at a 25 nM final concentration. A scrambled oligonucleotide and mock transfection were included as controls. Briefly, cells were seeded at 200.000 cells/well 24 hr before transfection. For transfections, cells were washed in Opti-Mem (Thermo Fisher Scientific cat. no. 51985-026) followed by 7-minute treatment of Lipofectamin in 900 μL Opti-Mem. Antisense oligonucleotides were added and cells incubated at 5% CO2 at 37° C. for 4 hours. Cells were washed once in Opti-Mem and 2.5 mL Dulbecco's MEM was then added to cells.

24 hours after transfection total RNA was isolated from the cells using the RNeasy mini kit (Qiagen) according to the manufacturer's instructions and 1 μg total RNA was reverse transcribed into cDNA using the High Capacity cDNA reverse transcription kit (Life Technologies cat. no. 4374967) according to the protocol provided by the manufacturer.

Target mRNA levels were determined by quantitative PCR using Taqman Gene Expression Master Mix (ABI cat. no. 4369542) and pre-designed Taqman assays for PDL1 (CD274) (IDT cat. no. Hs.PT.58.4665575), PDL2 (PDCD1LG2) (IDT cat. no. Hs.PT.58.21416962), and IDO1 (IDT cat. no. Hs.PT.58.924731) furthermore the expression of TBP mRNA was measured (IDT cat. no. Hs.PT.58v.39858774) and used as an endogenous control in calculation of expression changes using the ΔΔCt method with efficiency correction. Values were normalized to Mock.

Quantitative PCR was carried out on a Quantstudio 6 Flex Real-Time thermocycler (ABI)

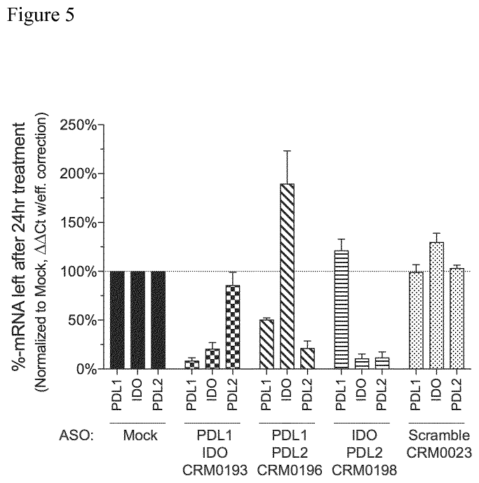

Examples of bispecific antisense oligonucleotide-mediated knockdown of PDL1/IDO1, PDL1/PDL2 and PDL2/IDO1 in GMS-10 cells are shown in FIG. 5.

Example 9. Antisense-Mediated Downregulation of Immune Checkpoint Proteins in Cultured Cancer Cells Using Bispecific Antisense Oligonucleotides

Human glioblastoma cell line GMS-10 (DSMZ cat. no. ACC405) was purchased from Leibniz Institue DSMZ-German Collection of Microorganisms and Cell Cultures and maintained in 85-90% Dulbecco's MEM (Sigma cat. no. D6546), 10-15% fetal bovine serum (Sigma cat. no. F2442), 2 mM L-glutamine (Sigma cat. no. G7513), and penicillin/streptomycin (Sigma cat. no. P4333) in a humidified 5% CO2 incubator at 37° C. and passaged twice a week.

For transfection of the immune checkpoint antisense oligonucleotides listed in Table 7.1 or 7.2, GMS-10 cells were seeded in 6-well cell culture plates and transfected using 5 μL/mL Lipofectamine 2000 (Thermo Fisher Scientific cat. no. 11668027) using antisense oligonucleotides at a 25 nM final concentration. A scrambled oligonucleotide and mock transfection were included as controls. Briefly, cells were seeded at 200.000 cells/well 24 hr before transfection. For transfections, cells were washed in Opti-Mem (Thermo Fisher Scientific cat. no. 51985-026) followed by 7-minute treatment of Lipofectamin in 900 μL Opti-Mem. Antisense oligonucleotides were added and cells incubated at 5% CO2 at 37° C. for 4 hours. Cells were washed once in Opti-Mem and 2.5 mL Dulbecco's MEM was then added to cells.

48 hours after transfection total protein was isolated from the cells scrapped from the well. Cells were lysed in RIPA buffer supplemented with complete proteinase inhibitor cocktail (Sigma cat. no. 000000011697498001). Cells were passed through a syringe ten times to ensure efficient lysis. Cell debris was removed by a ten-minute centrifugation at 8000×g.

Protein levels were assessed by western blotting. Proteins samples were denatured in NuPAGE LDS sample buffer (Invitrogen cat. no. NP0007) with NuPAGE reducing agent (Invitrogen cat. no. NP0004). Proteins were separated on Mini-PROTEAN TGX gels (Bio Rad cat. no. 456,8123) in TGS running buffer (Bio Rad cat. no. 161-0732).

Proteins were transferred to a nitrocellulose membrane using Trans-Blot Turbo transfer packs (Bio Rad cat. no. 170-4159). Membranes were blocked with TBS Tween (Thermo Scientific cat. no. 28360) supplemented with 5% skimmed milk powder (Sigma cat. no. 70166). Antibody incubation was performed in TBS tween with 5% skimmed milk powder. The following antibodies were used: 1) PDL1 antibody (1:1000, Abcam cat. no. ab213524) and secondary anti-rabbit antibody (1:10000, Dako cat. no. P0448). Vinculin was used as loading control; the following antibodies were used (Vinculin antibody 1:2000, Sigma cat. no. V9131 and secondary anti-mouse antibody, 1:10000, Dako cat. no. P0447). Protein bands were visualized by Clarity western ECL substrate (Bio Rad cat. no. 170-5060).

Gel electrophoresis was done in a Mini-PROTEAN® Tetra Vertical system (Bio Rad cat. no. 1658004). Blotting was carried out in a Trans-Blot® Turbo™ Transfer System (Bio Rad cat. no. 1704150). Blots were develop using a ChemiDoc™ Imaging System (Bio Rad cat. no. 17001401)

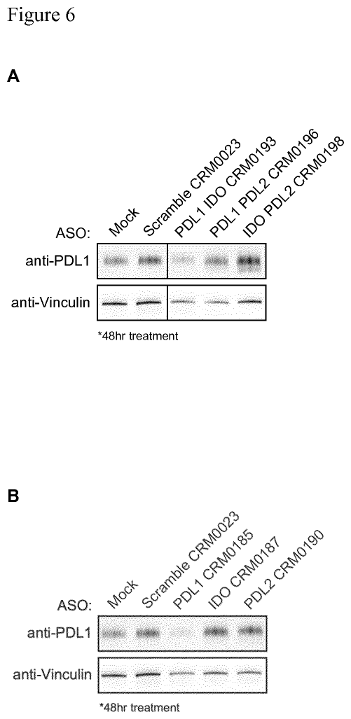

Examples of PDL1 protein downregulation in GMS-10 cells are shown in FIG. 6A.

Example 10. Antisense-Mediated Knockdown of Immune Checkpoint-Encoding mRNAs in Cultured Cancer Cells Using Monospecific Antisense Oligonucleotides

Human glioblastoma cell line GMS-10 (DSMZ cat. no. ACC405) was purchased from Leibniz Institue DSMZ-German Collection of Microorganisms and Cell Cultures and maintained in 85-90% Dulbecco's MEM (Sigma cat. no. D6546), 10-15% fetal bovine serum (Sigma cat. no. F2442), 2 mM L-glutamine (Sigma cat. no. G7513), and penicillin/streptomycin (Sigma cat. no. P4333) in a humidified 5% CO2 incubator at 37° C. and passaged twice a week.

For transfection of the immune checkpoint antisense oligonucleotides CRM0185, CRM0187 and CRM0190 (SEQ ID Nos: 1597, 1653 and 1625 respectively) listed in Table 7.1, GMS-10 cells were seeded in 6-well cell culture plates and transfected using 5 μL/mL Lipofectamine 2000 (Thermo Fisher Scientific cat. no. 11668027) using antisense oligonucleotides at a final concentration of 25 nM. A scrambled oligonucleotide (CRM0023) and mock transfection were included as controls. Briefly, cells were seeded at 200.000 cells/well 24 hr before transfection. For transfections, cells were washed in Opti-Mem (Thermo Fisher Scientific cat. no. 51985-026) followed by 7-minute treatment with Lipofectamin in 900 μL Opti-Mem. Antisense oligonucleotides were added and cells incubated at 5% CO2 at 37° C. for 4 hours. Cells were washed once in Opti-Mem and 2.5mL Dulbecco's MEM was then added to cells.

24 hours after transfection, total RNA was isolated from the cells using the RNeasy mini kit (Qiagen) according to the manufacturer's instructions and 1 μg total RNA was reverse transcribed into cDNA using the High Capacity cDNA reverse transcription kit (Life Technologies cat. no. 4374967) according to the protocol provided by the manufacturer.

Target mRNA levels were determined by quantitative PCR using Taqman Gene Expression Master Mix (ABI cat. no. 4369542) and pre-designed Taqman assays for PDL1 (CD274) (IDT cat. no. Hs.PT.58.4665575), PDL2 (PDCD1LG2) (IDT cat. no. Hs.PT.58.21416962), and IDO1 (IDT cat. no. Hs.PT.58.924731). Furthermore, the expression of TBP mRNA was measured (IDT cat. no. Hs.PT.58v.39858774) and used as an endogenous control in calculation of changes in expression of the target genes, using the ΔΔCt method with efficiency correction. Values were normalized to Mock.

Quantitative PCR was carried out on a Quantstudio 6 Flex Real-Time thermocycler (ABI)

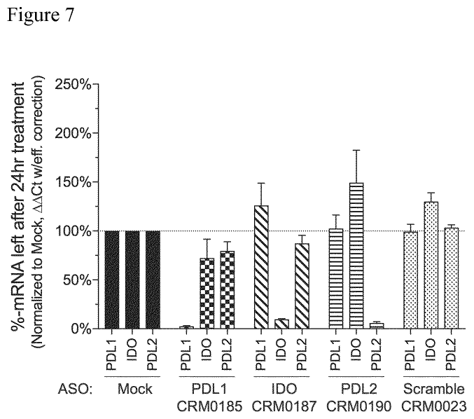

Examples of PDL1, IDO1, and PDL2 mRNA knockdown in GMS-10 cells are shown in FIG. 7.

Example 11. Antisense-Mediated Downregulation of Immune Checkpoint Proteins in Cultured Cancer Cells Using Monospecific Antisense Oligonucleotides

GMS-10 cells were maintained and transfected with antisense oligonucleotides CRM0185, CRM0187, and CRM0190 as described in Example 10.

48 hours after transfection total protein was isolated from the cells scraped from the well. Cells were lysed in RIPA buffer supplemented with complete proteinase inhibitor cocktail (Sigma cat. no. 000000011697498001). Cells were passed through a syringe ten times to ensure efficient lysis. Cell debris was removed by a ten-minute centrifugation at 8000×g.

Protein levels were assessed by western blotting. Protein samples were denatured in NuPAGE LDS sample buffer (Invitrogen cat. no. NP0007) with NuPAGE reducing agent (Invitrogen cat. no. NP0004). Proteins were separated on Mini-PROTEAN TGX gels (Bio Rad cat. no. 456,8123) in TGS running buffer (Bio Rad cat. no. 161-0732).

Proteins were transferred to a nitrocellulose membrane using Trans-Blot Turbo transfer packs (Bio Rad cat. no. 170-4159). Membranes were blocked in TBS-Tween (Thermo Scientific cat. no. 28360) supplemented with 5% skimmed milk powder (Sigma cat. no. 70166). Antibody incubation was performed in TBS tween with 5% skimmed milk powder. The following antibodies were used: PDL1 antibody (1:1000, Abcam cat. no. ab213524) and secondary anti-rabbit antibody (1:10000, Dako cat. no. P0448). Vinculin was used as loading control. The following antibodies were used: Vinculin antibody (1:2000, Sigma cat. no. V9131) and secondary anti-mouse antibody (1:10000, Dako cat. no. P0447). Protein bands were visualized by Clarity western ECL substrate (Bio Rad cat. no. 170-5060).

Gel electrophoresis was done in a Mini-PROTEAN® Tetra Vertical system (Bio Rad cat. no. 1658004). Blotting was carried out in a Trans-Blot® Turbo™ Transfer System (Bio Rad cat. no. 1704150). Blots were develop using a ChemiDoc™ Imaging System (Bio Rad cat. no. 17001401)

Examples of PDL1 protein downregulation in GMS-10 cells are shown in FIG. 6B.

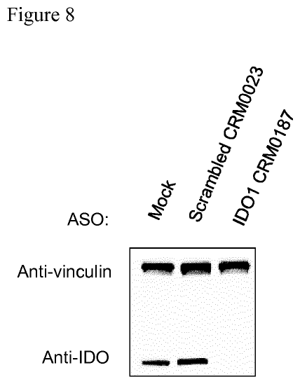

Examples of IDO1 protein downregulation in GMS-10 cells are shown in FIG. 8

Example 12. Antisense-Mediated Knockdown of Immune Checkpoint mRNAs in Cultured Cancer Cells Using Unassisted Uptake of Monospecific Antisense Oligonucleotides.

GMS-10 cells were maintained as described in Example 10. For unassisted uptake of the immune checkpoint antisense oligonucleotides CRM0185, CRM0187, and CRM0190, GMS-10 cells were seeded in 6-well cell culture and stimulated with 20 ng/mL IFN-γ to upregulate the immune checkpoint genes. 24 hours post-seeding media was changed and 20 ng/mL IFN-γ and antisense oligonucleotides were added at a final concentration of 2.5 μM. A scrambled oligonucleotide (CRM0023) and a mock were included as controls. Briefly, cells were seeded in a concentration of 80.000 cells/well and incubated at 5% CO2 at 37° C. for 4 hours. 20 ng/mL IFN-γ was added. 24 hr post-seeding antisense oligonucleotides and IFN-γ were added to fresh media and added to cells.

72 hours after antisense oligonucleotides were added, total RNA was isolated from the cells using the RNeasy mini kit (Qiagen) according to the manufacturer's instructions and 1 μg total RNA was reverse transcribed into cDNA using the High Capacity cDNA reverse transcription kit (Life Technologies cat. no. 4374967) according to the protocol provided by the manufacturer.

Target mRNA levels of PDL1, PDL2, IDO1, and TBP were determined by quantitative PCR as described in Example 10.

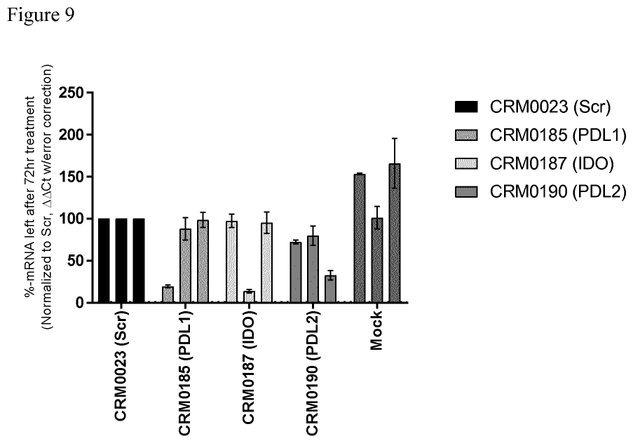

Examples of knockdown of PDL1, IDO, and PDL2 mRNAs in GMS-10 following unassisted uptake are shown in FIG. 9.

Example 13. Antisense-Mediated Downregulation of Immune Checkpoint Proteins in Cultured Cancer Cells Using Monospecific Antisense Oligonucleotides

Oligonucleotides CRM0185, CRM0187, and CRM0190 were delivered to GMS-10 cells by unassisted uptake, as described in Example 12.

72 hours after antisense oligonucleotides were added total protein was isolated and analyzed by Western blot as described in Example 11.

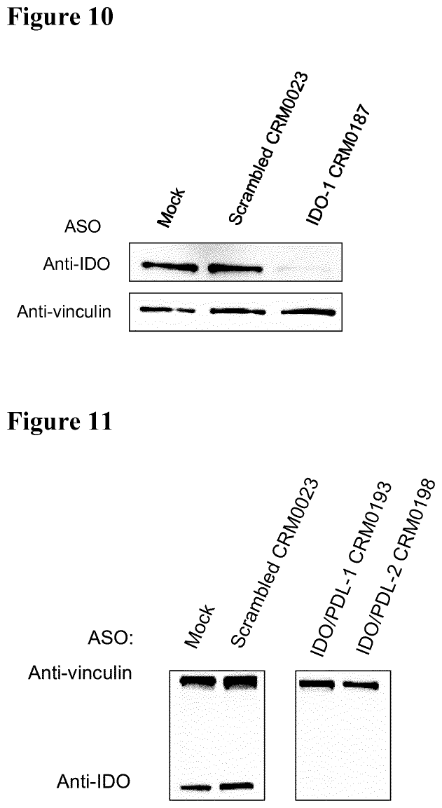

Examples of IDO1 protein down-regulation in GMS-10 following unassisted delivery of oligonucleotides are shown in FIG. 10.

Example 14. Antisense-Mediated Knockdown of Immune Checkpoint mRNAs in Cultured Cancer Cells Using Bispecific Antisense Oligonucleotides

Bispecific antisense oligonucleotides CRM0193, CRM0196, and CRM0198 (SEQ.ID.NO 377, 382, and 1154, respectively) were transfected Lipofectamine 2000 into GMS-10 cells, and the effect on expression levels of PDL1, IDO1, and PDL2 mRNA was measured by qPCR using the methods described in Example 10.

Examples of knockdown of PDL1, IDO, and PDL2 mRNAs in GMS-10 cells following transfection of bispecific antisense oligonucleotides are shown in FIG. 5.

Example 15. Antisense-Mediated Downregulation of Immune Checkpoint Proteins in Cultured Cancer Cells Using Bispecific Antisense Oligonucleotides

The bispecific antisense oligonucleotides were transfected into GMS-10 cells as described in Example 14.

48 hours after transfection, total protein was isolated and analyzed by western blot, as described in Example 11.

Examples of IDO1 protein downregulation using bispecific antisense oligonucleotides transfected into GMS-10 cells are shown in FIG. 11.

Example 16. Antisense-Mediated Knockdown of Immune Checkpoint mRNAs in Cultured Cancer Cells Using Antisense Oligonucleotides Targeting Both Human and Mouse Immune Checkpoint Proteins

Human glioblastoma cell line GMS-10 was maintained as described in Example 10. The murine glioblastoma cell line Neuro2a (N2a) was maintained in 85-90% Dulbecco's MEM (Sigma cat. no. D6546), 10-15% fetal bovine serum (Sigma cat. no. F2442), and penicillin/streptomycin (Sigma cat. no. P4333) in a humidified 5% CO2 incubator at 37° C. and passaged twice a week.

For transfection of the immune checkpoint-targeting antisense oligonucleotides CRM0129, CRM0131, CRM0134, CRM0135, CRM0138, and CRM0139 (SEQ.ID.NOs 1640, 1642, 1645, 1646, 1649, 1650) listed in Table 7.1, GMS-10 and N2A cells were seeded in 6-well cell culture plates and transfected using 5 μL/mL Lipofectamine 2000 (Thermo Fisher Scientific cat. no. 11668027) using antisense oligonucleotides at a 25 nM concentration. A scrambled oligonucleotide (CRM0023) and mock transfection were included as controls. Briefly, GMS-10 and N2A cells were seeded in a concentration of 120.000 and 250.000 cells/well, respectively, 24 hr before transfection. At transfections, cells were washed in Opti-Mem (Thermo Fisher Scientific cat. no. 51985-026) followed by 7-minute treatment of Lipofectamin in 900 μL Opti-Mem. Antisense oligo was added and cells incubated at 5% CO2 at 37° C. for 4 hours. Cells were washed once in Opti-Mem and 2.5 mL Dulbecco's MEM was then added to cells.

48 hours after transfection, total RNA was isolated from the cells using the RNeasy mini kit (Qiagen) according to the manufacturer's instructions and 1 μg total RNA was reverse transcribed into cDNA using the High Capacity cDNA reverse transcription kit (Life Technologies cat. no. 4374967) according to the protocol provided by the manufacturer.

Target mRNA levels were determined by quantitative PCR using Taqman Gene Expression Master Mix (ABI cat. no. 4369542) and pre-designed Taqman assays for PDL1 (CD274) (IDT cat. no. Hs.PT.58.4665575), PDL2 (PDCD1LG2) (IDT cat. no. Hs.PT.58.21416962), and IDO (IDT cat. no. Hs.PT.58.924731). Furthermore the expression of TBP mRNA was measured (IDT cat. no. Hs.PT.58v.39858774) and used as an endogenous control in calculation of expression changes using the ΔΔCt method with efficiency correction. Values were normalized to Scr-CRM0023.

Target mRNA levels in murine Neuro2a cells were determined by quantitative PCR using pre-designed Taqman assays for PDL1 (CD274) (IDT cat. no. Mm.PT.58.11921659), PDL2 (PDCD1LG2) (IDT cat. no. Mm.PT.58.11776803), and IDO (IDT cat. no. Mm.PT.58.29540170). Furthermore the expression of TBP mRNA was measured (IDT cat. no. mm.PT.39a.22214839) and used as an endogenous control in calculation of expression changes using the ΔΔCt method with efficiency correction. Values were normalized to Scr-CRM0023.

Quantitative PCR was carried out on a Quantstudio 6 Flex Real-Time thermocycler (ABI).

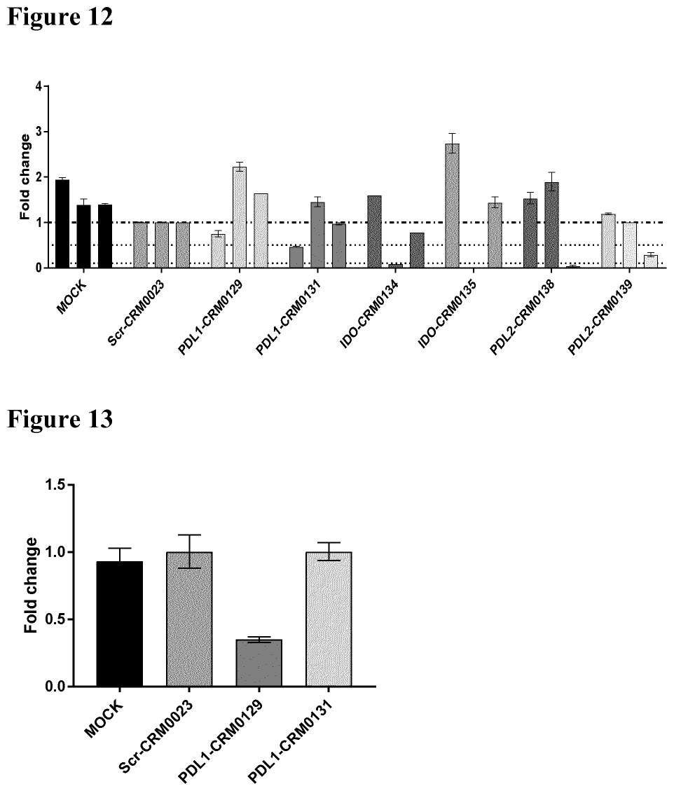

Examples of inhibition of PDL1, IDO, and PDL2 mRNAs in GMS-10 cells are shown in FIG. 12. Example of inhibition of PDL1 in Neuro-2a cells is shown in FIG. 13.

Example 17. Antisense-Mediated Downregulation of Immune Checkpoint Proteins in Cultured Cancer Cells Using Antisense Oligonucleotides Targeting Both Human and Mouse Immune Checkpoint Proteins

The antisense oligonucleotides CRM0129, CRM0131, CRM0134, CRM0135, CRM0138, and CRM0139 (SEQ.ID.NOs 1640, 1642, 1645, 1646, 1649, 1650) were transfected into GSM-10 cells and analysis of IDO1 protein levels were carried out as described in Examples 10 and 11.

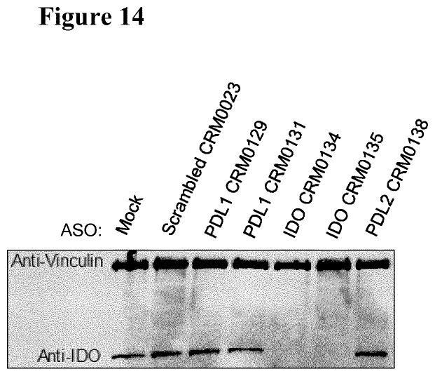

Examples of IDO1 protein downregulation in GMS-10 cells are shown in FIG. 14.

1. An antisense oligonucleotide consisting of a sequence of 14-22 nucleobases in length that is a gapmer comprising a central region of 6 to 16 consecutive DNA nucleotides flanked in each end by wing regions each comprising 1 to 5 nucleotide analogues, wherein the antisense oligonucleotide is complementary to an mRNA encoding an immune checkpoint protein.

2-57. (canceled)

58. The antisense oligonucleotide according to claim 1, wherein said antisense oligonucleotide comprises 1 to 21 phosphorothioate internucleotide linkages.

59. The antisense oligonucleotide according to claim 1, wherein the immune checkpoint protein is anyone selected from CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, or KIR2DL3.

60. The antisense oligonucleotide according to claim 1, wherein said oligonucleotide hybridizes to at least one mRNA selected from the group consisting of an mRNA encoding CD274, an mRNA encoding PDCD1LG2, an mRNA encoding CD80, an mRNA encoding CD86, an mRNA encoding CD276, an mRNA encoding VTCN1, an mRNA encoding TNFRSF14, an mRNA encoding LGALS9, an mRNA encoding IDO1, mRNA encoding HMOX1, an mRNA encoding PDCD1, an mRNA encoding CTLA4, an mRNA encoding LAG3, an mRNA encoding HAVCR2, an mRNA encoding TDO2, an mRNA encoding TIGIT, an mRNA encoding VSIR, an mRNA encoding CEACAM1, an mRNA encoding NTSE, an mRNA encoding KIR2DL1, and an mRNA encoding KIR2DL3.

61. The antisense oligonucleotide according to claim 1, wherein said oligonucleotide hybridizes to at least two mRNAs selected from the group consisting of an mRNA encoding CD274, an mRNA encoding PDCD1LG2, an mRNA encoding CD80, an mRNA encoding CD86, an mRNA encoding CD276, an mRNA encoding VTCN1, an mRNA encoding TNFRSF14, an mRNA encoding LGALS9, an mRNA encoding IDO1, mRNA encoding HMOX1, an mRNA encoding PDCD1, an mRNA encoding CTLA4, an mRNA encoding LAG3, an mRNA encoding HAVCR2, an mRNA encoding TDO2, an mRNA encoding TIGIT, an mRNA encoding VSIR, an mRNA encoding CEACAM1, an mRNA encoding NT5E, an mRNA encoding KIR2DL1, and an mRNA encoding KIR2DL3.

62. The antisense oligonucleotide according to claim 1, wherein said oligonucleotide hybridizes to a region of at least three mRNAs selected from the group consisting of an mRNA encoding CD274, an mRNA encoding PDCD1LG2, an mRNA encoding CD80, an mRNA encoding CD86, an mRNA encoding CD276, an mRNA encoding VTCN1, an mRNA encoding TNFRSF14, an mRNA encoding LGALS9, an mRNA encoding IDO1, mRNA encoding HMOX1, an mRNA encoding PDCD1, an mRNA encoding CTLA4, an mRNA encoding LAG3, an mRNA encoding HAVCR2, an mRNA encoding TDO2, an mRNA encoding TIGIT, an mRNA encoding VSIR, an mRNA encoding CEACAM1, an mRNA encoding NT5E, an mRNA encoding KIR2DL1, and an mRNA encoding KIR2DL3.

63. The antisense oligonucleotide according to claim 1, wherein the antisense oligonucleotide reduces expression of at least two immune checkpoint proteins selected from CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, or KIR2DL3.

64. The antisense oligonucleotide according to claim 1, wherein the antisense oligonucleotide reduces expression of three immune checkpoint proteins selected from CD274, PDCD1LG2, CD80, CD86, CD276, VTCN1, TNFRSF14, LGALS9, IDO1, HMOX1, PDCD1, CTLA4, LAG3, HAVCR2, TDO2, TIGIT, VSIR, CEACAM1, NT5E, KIR2DL1, or KIR2DL3.

65. The antisense oligonucleotide according to claim 1, wherein the antisense oligonucleotide is complementary to anyone of SEQ ID NOs: 1-375, or to anyone of SEQ ID NOs: 1473-1503, or to anyone of SEQ ID NOs: 1535-1593 or to SEQ ID NO: 1654 or to anyone of SEQ ID NOs: 1655-2001, or to anyone of SEQ ID NOs: 3044-3052, or to anyone of SEQ ID NOs: 3062-3097.

66. The antisense oligonucleotide according to claim 1, wherein at least one of the wing regions comprises at least one nucleoside analogue selected from beta-D-oxy LNA, alpha-L-oxy-LNA, beta-D-amino-LNA, alpha-L-amino-LNA, beta-D-thio-LNA, alpha-L-thio-LNA, 5′-methyl-LNA, beta-D-ENA alpha-L-ENA, tricyclo-DNA, 2′-fluoro, 2′-O-methyl, 2′-methoxyethyl (2′-MOE), 2′cyclic ethyl (cET), or Conformationally Restricted Nucleoside (CRN).

67. The antisense oligonucleotide according to claim 1, wherein at least one of the wing regions comprises two or more nucleoside analogues, wherein said nucleotide analogues is a mixture of LNA and at least one nucleoside analogue independently selected from the group consisting of tricyclo-DNA, 2′-fluoro, 2′-O-methyl, 2′-methoxyethyl (2′-MOE), 2′cyclic ethyl (cET), and Conformationally Restricted Nucleoside (CRN).

68. The antisense oligonucleotide according to claim 1, wherein at least one of the wing regions comprises a mixture of two or more nucleoside analogues selected from LNA or 2′-fluoro.

69. The antisense oligonucleotide according to claim 1, wherein the antisense oligonucleotide is any one of SEQ ID NOs: 376-1472, or anyone of SEQ ID NOs: 1504-1534, or anyone of SEQ ID NOs: 1594-1653, or anyone of SEQ ID NOs: 2002-3043, or anyone of SEQ ID NOs: 3053-3061, or anyone of SEQ ID NOs: 3098-3133.

70. The antisense oligonucleotide according to claim 1, wherein the antisense oligonucleotide is conjugated with a ligand.

71. The antisense oligonucleotide according to claim 1, wherein the antisense oligonucleotide is conjugated with folic acid or N-acetylgalactosamine (GalNAc).

72. The antisense oligonucleotide according to claim 1, wherein the antisense oligonucleotide is unconjugated.

73. A pharmaceutical composition comprising 1, 2, 3, 4, 5, 6, 7, 8, 9 or 10 antisense oligonucleotides according to claim 1, wherein the antisense oligonucleotides are selected so that the composition targets at least two immune checkpoint proteins.

74. A method inducing tumor regression in a human, comprising administration of a therapeutically effective dose of the antisense oligonucleotide according to claim 1 to a human.

75. The method of claim 74, comprising:
a. Isolating tumor-specific T-cells from a cancer patient;
b. Expanding the T-cells ex vivo;
c. Modifying the T-cells by reducing expression of one or more of immune checkpoint proteins selected from CTLA4, PDCD1, LAG3, HAVCR2, TIGIT, or CEACAM1 in the T-cells by providing one or more of the antisense oligonucleotides of claim 1; and
d. Administering the modified T-cells to the cancer patient.

76. The method of claim 74, comprising:
a. Isolating dendritic cells from a cancer patient;
b. Testing the dendritic cells for expression of an immune checkpoint protein selected from CD274, PDCD1LG2, CD80, CD86, CD276, VTCN1, TNFRSF14, LGALS9, IDO1, HMOX1, TDO2, VSIR, or NT5E;
c. Expanding the dendritic cells ex vivo;
d. Modifying the dendritic cells by reducing expression of one or more of the immune checkpoint proteins for which the dendritic cells tested positive by providing one or more of the antisense oligonucleotides of claim 1; and
e. Administering the modified dendritic cells to the cancer patient.

77. The method of claim 74, comprising:
a. Isolating T-cells from a cancer patient;
b. Expanding the T-cells ex vivo;
c. Co-culturing the T-cells with modified dendritic cells or non-modified dendritic cells or other antigen presenting cells;
d. Modifying the T-cells by reducing expression of one or more of immune checkpoint proteins selected from CTLA4, PDCD1, LAG3, HAVCR2, TIGIT, or CEACAM1 in the T-cells by providing one or more of the antisense oligonucleotides of claim 1; and
e. Administering the modified T-cells to the cancer patient.

78. The method of claim 74, comprising:
a. Isolating NK cells from a cancer patient;
b. Expanding the NK cells ex vivo;
c. Testing the NK cells for expression of an immune checkpoint protein selected from KIR2DL1 or KIR2DL3;
d. Modifying the NK cells by reducing expression of one or more of the immune checkpoint proteins for which the NK cells tested positive by providing one or more of the antisense oligonucleotides of claim 1 to the NK cells; and
e. Administering the modified NK cells to the cancer patient.
